(19) United States
(12) Patent Application Publication
(10) Pub. No.: US 2025/0333542 A1
(43) Pub. Date: Oct. 30, 2025

(54) INTERFERON GAMMA VARIANTS AND ANTIGEN BINDING MOLECULES COMPRISING THESE

(71) Applicant: Hoffmann-La Roche Inc., Little Falls, NJ (US)

(72) Inventors: Samuele CALABRO, Schlieren (CH); Lucia CAMPOS CARRASCOSA, Schlieren (CH); Stephan GASSER, Fislisbach (CH); Leo Frederik KUNZ, Basel (CH); Ekkehard MOESSNER, Kreuzlingen (CH); Evelyn SAUER, Ehrendingen (CH); Pablo UMAÑA, Lachen (CH); Dario VENETZ, Bonstetten (CH)

(73) Assignee: Hoffmann-La Roche Inc., Little Falls, NJ (US)

(21) Appl. No.: 18/898,564

(22) Filed: Sep. 26, 2024

Related U.S. Application Data
(63) Continuation of application No. PCT/EP2023/057751, filed on Mar. 27, 2023.

(30) Foreign Application Priority Data
Mar. 28, 2022 (EP) 22164773.8

(51) Int. Cl. C07K 16/40; A61K 38/00; A61K 39/00; A61P 35/00; C07K 14/57; C07K 16/24

(52) U.S. Cl. CPC C07K 16/40; A61P 35/00; C07K 14/57; C07K 16/249; A61K 38/00; A61K 2039/505; C07K 2317/565; C07K 2317/622; C07K 2317/71; C07K 2319/50; C07K 2319/70

(57) ABSTRACT

The invention relates to new antigen binding molecules comprising (i) an antibody that specifically binds to a tumor associated antigen and (ii) an interferon gamma (IFNG) variant polypeptide that terminates with the C-terminal amino acid sequence KRKRP (SEQ ID NO:1), to the new IFNG variant polypeptides included therein, to methods of producing these molecules and to methods of using the same.

### CROSS-REFERENCE TO RELATED APPLICATIONS

This application is a continuation of International Application No. PCT/EP2023/057751, filed Mar. 27, 2023, which claims benefit of priority to European Application No. 22164773.8, filed Mar. 28, 2022, each of which is incorporated herein by reference in its entirety.

### SEQUENCE LISTING

The instant application contains a sequence listing which has been submitted electronically in xml format and is hereby incorporated by reference in its entirety. Said .xml copy, created on Sep. 26, 2024, is named P37456-US_Sequence_Listing.xml and is 243,194 bytes in size and updated by a file titled P37456-US_Replacement_Sequence_Listing created on May 7, 2025, which is 243,190 bytes in size.

### FIELD OF THE INVENTION

The invention relates to new antigen binding molecules comprising (i) an antibody that specifically binds to a tumor-associated antigen and (ii) a new interferon gamma (IFNG) variant polypeptide that terminates with the C-terminal amino acid sequence KRKRP (SEQ ID NO: 1), in particular to antigen binding molecules comprising an antibody that specifically bind to Fibroblast activation protein (FAP) and the new IFNG variant polypeptide. In addition, the invention relates to the new IFNG variant polypeptides included therein, and to polynucleotide molecules encoding the antigen binding molecules or IFNG variant polypeptides, and vectors and host cells comprising such polynucleotide molecules. Further aspects of the invention are methods of producing these molecules and methods of using the same.

### BACKGROUND

In recent years the application of immunotherapy treatments to cancer has grown dramatically and cancer immunotherapy has become an important strategy to fight cancer. Immune checkpoint modulators including anti-PD-1 have been established as standard of care for many cancer types. However, despite all progressions that have been made in the last years by cancer immunotherapy treatments, several patients still do not respond to the available immunotherapies due to intrinsic or adaptive resistance mechanisms. In particular, it is becoming clearer that patients that do not respond to cancer immunotherapies are often characterised by a non-inflamed immune phenotype. In fact, a correlation has been shown between the immune cell infiltration in the tumor and the capacity of patients to respond to immunotherapy treatments. Overall, there is a clear medical need for patients to develop new therapies that aim to enhance the immunogenicity and to increase immune cell infiltration in tumors.

In parallel to the developments mentioned above, the interest for cytokines as a potential cancer treatment has drastically increased. Interferon gamma (IFNG or IFN-γ) is a cytokine that is mainly produced and secreted by activated lymphocytes like CD4⁺ and CD8⁺ T cells, as well as natural killer (NK) cells in response to inflammatory or immune stimuli. IFNG is a homodimer and its receptors (IFNGR1 and IFNGR2) are expressed across hematopoietic and non-hematopoietic cells. IFNGR1 is stably expressed on the cell surface whereas IFNGR2 is differentially expressed and used to regulate the IFNG signal. Binding of IFNG to its receptors induces recruitment and activation of the Janus kinases, JAK1 and JAK2, which phosphorylate and activate STAT1. After phosphorylation, STAT1 translocates to the nucleus where it binds specific promoters and modulates the transcription of IFNG-regulated genes.

In contrast to many cancer therapies that are on the market and under development, IFNG has the ability to act on both tumor cells and multiple immune cells including T cells and dendritic cells. The effects of IFNG on different cell types have several benefits, which include 1) enhanced expression of MHC-I molecules on the surface of both tumour cells and antigen-presenting cells, 2) the recruitment of immune cells to the tumour site through the induction of CXCL9, CXCL10 and CXCL11 production and 3) the ability to increase tumor antigen cross-presentation with a subsequent enhancement of an anti-tumor immune response. Beside all these effects IFNG plays a role in the generation of a Th1 environment, monocyte differentiation, macrophage polarisation and angiogenesis. Based on its cytostatic, pro-apoptotic and antiproliferative functions, IFNG is considered potentially useful in the therapy of cancer.

However, the receptors of IFNG are expressed on many different cell types and thus its activity may be diminished by a sink effect. IFNG can also show undesirable side effects. In addition, problems with administration, bioavailability and short half-life may arise. Thus, there is a need for new IFNG molecules that can selectively activate T cells in the tumor environment. WO 2017/139468 A1 discloses fusion proteins comprising a Her2-binding scFv and an interferon gamma monomer terminating with the amino acid sequence AKTGKRKRSQ (SEQ ID NO:127). However, there is still a need to provide molecules that possess a high stability to maintain the IFNG activity and that are more suitable for the administration to human patients.

### SUMMARY OF THE INVENTION

In order to overcome the current challenges, that are mainly linked to dose limiting toxicity and sink effect, we developed a tumor-targeting, masked IFNG that is inactive in circulation and in healthy tissues and active only at the tumor site eliciting the benefits described above. Delivery of active IFNG directly to the tumor environment will overcome the potential dose limitations of IFNG that is produced following the activation of T cells and NK cells and in particular, it will give the opportunity to be efficient also in immune desert tumors where at the moment there are more needs for a new cancer immunotherapy.

This invention thus provides a novel approach of targeting an IFNG variant with advantageous properties for immunotherapy directly to immune effector cells, such as cytotoxic T lymphocytes, rather than tumor cells, through conjugation of the IFNG variant to an antibody that binds to a tumor-associated antigen, in particular Fibroblast activation protein (FAP). This results in activation of T cells in the tumor microenvironment. Fibroblast activation protein (FAP) is a serine protease highly expressed on the cell surface of cancer-associated stroma cells, and on fibroblastic reticular cells in secondary lymphoid organs, but has otherwise very limited expression in normal tissues. FAP is highly prevalent in various cancer indications allowing its usage as targeting moiety for drugs that should accumulate within the tumor stroma.

The molecules of the invention comprise a homodimer of an interferon gamma (IFNG) variant polypeptide that terminates with the C-terminal amino acid sequence KRKRP (SEQ ID NO: 1). It has been shown herein that the presence of the KRKR patch, i.e. the amino acid sequence of KRKR (SEQ ID NO:78) is important for the activity of IFNG. The inventors found that in order to avoid proteolysis, the C-terminus of wild-type IFNG has to be stabilized with a proline cap so that the sequence of the IFNG variant polypeptide terminates with the amino acid sequence KRKRP (SEQ ID NO:1).

Thus, provided herein are antigen binding molecules, comprising
(i) an antibody that specifically binds to a tumor-associated antigen and
(ii) a homodimer of an interferon gamma (IFNG) variant polypeptide, wherein the IFNG variant polypeptide is characterized by the C-terminal amino acid sequence KRKRP (SEQ ID NO: 1), meaning that it terminates at the C-terminus with the amino acid sequence KRKRP (SEQ ID NO:1).

These antigen binding molecules thus comprise an antibody to which two identical interferon gamma (IFNG) variant polypeptides that in difference to wild type IFNG terminate with the amino acid sequence KRKRP (SEQ ID NO:1) have been fused. In one aspect, the interferon gamma (IFNG) variant polypeptide comprises the amino acid sequence of SEQ ID NO: 2 or SEQ ID NO:3. In one aspect, the interferon gamma (IFNG) variant polypeptide consists of the amino acid sequence of SEQ ID NO:2 or SEQ ID NO:3. In one particular aspect, the IFNG variant polypeptide comprises or consists of the amino acid sequence of SEQ ID NO: 2.

In one aspect, provided are antigen binding molecules, comprising (i) an antibody that specifically binds to a tumor-associated antigen and (ii) a homodimer of an interferon gamma (IFNG) variant polypeptide, wherein the IFNG variant polypeptide is characterized in that it terminates at the C-terminus with the amino acid sequence KRKRP (SEQ ID NO:1) and wherein the first IFNG variant polypeptide is fused with its N-terminus to the C-terminus of the first heavy chain and second IFNG variant polypeptide is fused with its N-terminus to the C-terminus of the second heavy chain, optionally via a linker.

In one aspect, the antibody that specifically binds to a tumor-associated antigen is an antibody that specifically binds to Fibroblast activation protein (FAP). In one aspect, provided is an antigen binding molecule, wherein the antibody that specifically binds to FAP comprises (a) a heavy chain variable region (V_{H}FAP) comprising a heavy chain complementary determining region (CDR-H1) comprising the amino acid sequence of SEQ ID NO:4, a CDR-H2 comprising the amino acid sequence of SEQ ID NO:5, and a CDR-H3 comprising the amino acid sequence of SEQ ID NO:6, and a light chain variable region (V_{L}FAP) comprising a light chain complementarity determining region (iv) CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9, or (b) a heavy chain variable region (V_{H}FAP) comprising a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO: 14, and a light chain variable region (V_{L}FAP) comprising a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17. In one aspect, provided is an antigen binding molecule, wherein the antibody that specifically binds to FAP comprises a heavy chain variable region (V_{H}FAP) comprising the amino acid sequence of SEQ ID NO:10 and a light chain variable region (V_{L}FAP) comprising the amino acid sequence of SEQ ID NO:11 or it comprises a heavy chain variable region (V_{H}FAP) comprising the amino acid sequence of SEQ ID NO: 18 and a light chain variable region (V_{L}FAP) comprising the amino acid sequence of SEQ ID NO:19. In one particular aspect, provided is an antigen binding molecule, wherein the antibody that specifically binds to FAP comprises a heavy chain variable region (V_{H}FAP) comprising the amino acid sequence of SEQ ID NO: 10 and a light chain variable region (V_{L}FAP) comprising the amino acid sequence of SEQ ID NO:11.

In one aspect, the antigen binding molecule comprises an Fc domain, in particular and IgG1 Fc domain or an IgG4 Fc domain. In one aspect, the Fc domain comprises one or more amino acid substitution that reduces the binding affinity of the antibody to an Fc receptor and/or effector function. In one aspect, the Fc domain is of human IgG1 subclass with the amino acid mutations L234A, L235A and P329G (EU numbering according to Kabat EU index). In another aspect, the Fc domain is a murine Fc domain and comprises the amino acid mutations D265A and P329G (EU numbering according to Kabat EU index).

In one aspect, the antigen binding molecule is protease-activatable and comprises a protease recognition site and a masking moiety. The antigen binding molecule thus comprises (i) an antibody that specifically binds to a tumor-associated antigen, (ii) a homodimer of an interferon gamma (IFNG) variant polypeptide, wherein the IFNG variant polypeptide is characterized in that it terminates at the C-terminus with the amino acid sequence KRKRP (SEQ ID NO:1), (iii) a protease recognition site and (iv) a masking moiety.

In one aspect, the protease recognition site is a substrate for matriptase. In one aspect, the protease recognition site comprises or consists of the amino acid sequence PQARK (SEQ ID NO: 20) or HQARK (SEQ ID NO:21). In one particular aspect, the protease recognition site comprises or consists of the amino acid sequence PQARK (SEQ ID NO:20). In one aspect, the protease recognition site is part of a cleavable peptide linker which connects the masking moiety with the IFNG variant polypeptide.

In one aspect, provided is an antigen binding molecule, wherein the masking moiety is fused at its N-terminus to the C-terminus of the IFNG variant polypeptide via the cleavable peptide linker (mask release format). In one aspect, the IFNG variant polypeptide is fused at its N-terminus via a stable linker to the C-terminus of the antibody.

In another aspect, provided is an antigen binding molecule, wherein the masking moiety is fused at its N-terminus to the C-terminus of the Fc domain via a stable linker and at its C-terminus to the N-terminus of the IFNG variant polypeptide via the cleavable peptide linker (cytokine release format).

In one aspect, the masking moiety is an antibody fragment that specifically binds to IFNG. In one particular aspect, the masking moiety is an scFv that specifically binds to IFNG.

In one aspect, the masking moiety that specifically binds to IFNG, in particular an scFv, comprises
(a) a heavy chain variable region (V_{H}IFNG) comprising a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a light chain variable region (V_{L}IFNG) comprising a light chain complementarity determining region (iv) CDR-L1 comprising the amino acid sequence of SEQ ID NO:25, a CDR-L2 comprising the amino acid sequence of SEQ ID NO:26, and a CDR-L3 comprising the amino acid sequence of SEQ ID NO:27, or
(b) a heavy chain variable region (V_{H}IFNG) comprising a CDR-H1 comprising the amino acid sequence of SEQ ID NO:30, a CDR-H2 comprising the amino acid sequence of SEQ ID NO:31, and a CDR-H3 comprising the amino acid sequence of SEQ ID NO:32, and a light chain variable region (V_{L}IFNG) comprising a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35.

In one aspect, the masking moiety that specifically binds to IFNG, in particular an scFv, comprises (a) a heavy chain variable region (V_{H} IFNG) comprising the amino acid sequence of SEQ ID NO:28 and a light chain variable region (V_{L} IFNG) comprising the amino acid sequence of SEQ ID NO:29, or (b) a heavy chain variable region (V_{H} IFNG) comprising the amino acid sequence of SEQ ID NO:36 and a light chain variable region (V_{L} IFNG) comprising the amino acid sequence of SEQ ID NO:37. In one particular aspect, the masking moiety that specifically binds to IFNG, in particular an scFv, comprises (a) a heavy chain variable region (V_{H} IFNG) comprising the amino acid sequence of SEQ ID NO:28 and a light chain variable region (V_{L} IFNG) comprising the amino acid sequence of SEQ ID NO:29.

In one particular aspect, provided herein is an antigen binding molecule containing an IFNG variant polypeptide, wherein said antigen binding molecule comprises
(i) two heavy chains comprising the amino acid sequence of SEQ ID NO:40 and two light chains comprising the amino acid sequence of SEQ ID NO:41, or
(ii) two heavy chains comprising the amino acid sequence of SEQ ID NO:42 and two light chains comprising the amino acid sequence of SEQ ID NO:41, or
(iii) two heavy chains comprising the amino acid sequence of SEQ ID NO:43 and two light chains comprising the amino acid sequence of SEQ ID NO:44, or
(iv) two heavy chains comprising the amino acid sequence of SEQ ID NO:45 and two light chains comprising the amino acid sequence of SEQ ID NO:44.

Furthermore, provided herein is an interferon gamma (IFNG) variant polypeptide, wherein the IFNG variant polypeptide is characterized by the C-terminal amino acid sequence KRKRP (SEQ ID NO:1). In one aspect, the IFNG variant polypeptide is a human IFNG variant polypeptide and comprises or consists of the amino acid sequence of SEQ ID NO:2. In another aspect, the IFNG variant polypeptide is a mouse IFNG variant polypeptide and comprises or consists of the amino acid sequence of SEQ ID NO:3.

According to another aspect of the invention, there is provided isolated one or more isolated polynucleotide encoding an antigen binding molecule as described herein before. Also provided is an isolated polynucleotide encoding an IFNG variant polypeptide as described herein before. The invention further provides a vector, particularly an expression vector, comprising the isolated polynucleotides of the invention and a host cell comprising the isolated nucleic acids or the expression vector of the invention. In some aspects the host cell is an eukaryotic cell, particularly a mammalian cell. In some aspects, the host cell a prokaryotic cell. In another aspect, provided is a method of producing an antigen binding molecule or an IFNG variant polypeptide as described herein before, comprising culturing the host cell as described above under conditions suitable for the expression of the antigen binding molecule or the an IFNG variant polypeptide, and isolating the antigen binding molecule or the an IFNG variant polypeptide. The invention also encompasses the antigen binding molecule that comprises an IFNG variant polypeptide or the IFNG variant polypeptide produced by the method described herein.

The invention further provides a pharmaceutical composition comprising an antigen binding molecule as described herein before or the IFNG variant polypeptide as described herein before and a pharmaceutically acceptable carrier. In one aspect, the pharmaceutical composition comprises an additional therapeutic agent. In one aspect, the pharmaceutical composition is for the treatment of a disease. In one particular aspect, the disease is cancer.

Also encompassed by the invention is the antigen binding molecule or an IFNG variant polypeptide as described herein before, or the pharmaceutical composition comprising the antigen binding molecule or an IFNG variant polypeptide, for use as a medicament.

In one aspect, provided is an antigen binding molecule as described herein before or an IFNG variant polypeptide as described herein before or the pharmaceutical composition of the invention, for use in the treatment of cancer. In another specific aspect, the invention provides the antigen binding molecule or an IFNG variant polypeptide as described herein before for use in the treatment of cancer, wherein the antigen binding molecule or IFNG variant polypeptide is administered in combination with a chemotherapeutic agent, radiation and/or other agents for use in cancer immunotherapy.

In a further aspect, the invention provides a method of inhibiting the growth of tumor cells in an individual comprising administering to the individual an effective amount of the antigen binding molecule or an IFNG variant polypeptide as described herein before, or the pharmaceutical composition of the invention, to inhibit the growth of the tumor cells. In another aspect, the invention provides a method of treating a disease in an individual, comprising administering to said individual a therapeutically effective amount of the antigen binding molecule or an IFNG variant polypeptide as described herein before, or the pharmaceutical composition of the invention. In a specific aspect, the disease is cancer.

Also provided is the use of the antigen binding molecule as described herein before for the manufacture of a medicament for the treatment of a disease in an individual in need thereof, in particular for the manufacture of a medicament for the treatment of cancer. In a specific aspect, the disease is cancer. In any of the above aspects the individual is a mammal, particularly a human.

### BRIEF DESCRIPTION OF THE DRAWINGS

FIGS. 1A to 1C provide an overview of the engineering of the IFNG C-terminal sequence. In FIG. 1A a schematic scheme of the format of the IFNG molecules prepared in Example 1 is shown. IFNG is fused to the C-termini of the heavy chains of a targeting IgG antibody. The IgG-fused IFNG wild-type C-terminus is prone to proteolytic degradation leading to a truncation variant inactive in signaling. In FIG. 1B IFNG C-terminal sequences with deletion of certain C-terminal amino acids are shown. For the deletion series, C-terminal residues were gradually removed, and then replaced with other amino acid to provide a cap. The KRKR sequence is important for the activity and has to be conserved FIG. 1C gives an overview of the amino acid mutations that were introduced in the KRKR sequence in the mutation series as described in Example 1. FIG. 1D shows a schematic scheme of the mask-release format. IFNG is fused to the C-terminus of the heavy chain of the targeting IgG via a stable linker (Linker 1). To create a masked format a IFNG-specific scFv-domain is fused via a cleavable linker (Linker 2 with PQARK cleavage site) to the C-terminus of IFNG. FIG. 1E shows a schematic scheme of the cytokine-release format. IFNG is fused via a cleavable linker (Linker 2 with PQARK cleavage site) to the C-terminus of a IFNG-specific scFv-domain which is fused via a stable linker (Linker 1) to the heavy chain of the targeting IgG.

FIGS. 2A to 2C show the Signalling activity of human IFNG deletion series variants measured on HEK-blue IFNG reporter cells. In FIG. 2A the signalling activity of wild-type IFNG (P1AF3568), inactive variant (P1AF3569) and molecules with IFNG variants described in the literature (Slodowski et al.) is compared. FIG. 2B compares the wild-type IFNG molecule (P1AF3568) with molecule containing proline capped IFNG variants. FIG. 2C compares the wild-type IFNG molecule (P1AF3568) with molecule containing serine-proline capped IFNG variants.

FIGS. 3A to 3C show the Signalling activity of human IFNG mutation series variants measured on HEK-blue IFNG reporter cells. In FIG. 3A the signalling activity of the IFNG variant with C-terminal KRKRP (P1AF3574) is compared with IFNG variant molecules, wherein one or two amino acids in the KRKR patch have been replaced by glutamate. FIG. 3B compares the signalling activity of the IFNG variant with C-terminal KRKRP (P1AF3574) with IFNG variant molecules, wherein one or two amino acids in the KRKR patch have been replaced by proline. FIG. 3C compares the signalling activity of the IFNG variant with C-terminal KRKRP (P1AF3574) with IFNG variant molecules, wherein one or two amino acids in the KRKR patch have been replaced by serine.

FIG. 4A shows a multiple sequence alignment of human and murine wild-type IFNG as well as engineered IFNG variants. The aligned sequences shown are the consensus sequence (SEQ ID NO:125), human IFNG KRKRP 1-132 (SEQ ID NO:2) murine IFNG KRKRP 22-153 (SEQ ID NO:3), human wild-type IFNG (SEQ ID NO:60) and mouse wild-type IFNG (SEQ ID NO: 61). FIG. 4B shows the induction of MHC-I on murine MC38-huCEA tumor cell lines in response to treatment for two days with FAP-targeted, anti murine IFNG-scFv masked IFNG compounds containing a cleavable PQARK linker (with and without Matriptase) in comparison to the activity of FAP-targeted, unmasked IFNG. FIG. 4C shows the induction of PD-L1 on murine MC38-huCEA tumor cell lines in response to treatment for two days with FAP-targeted, anti murine IFNG-scFv masked IFNG compounds containing a cleavable PQARK linker (with and without Matriptase) in comparison to the activity of FAP-targeted, unmasked IFNG.

FIG. 5 shows the study layout highlighting the key events of the in vivo study conducted to test the activity of P1AG3755 in vivo. The KPC4662-huCEA tumor model is an excluded tumor model meaning that CD8 T cells are only present in the periphery of the tumor. MHC-I and PD-L1 expression are low at baseline. P1AG3755 was given once at two different doses to observe changes in MHC-I, PD-L1 expression and CD8 infiltration.

FIG. 6A and FIG. 6B show the MHC-I expression in cancer cells (FIG. 6A) and fibroblasts (FIG. 6B) at different time points of the study. MHC-I expression is statistically significantly increased on cancer cells and fibroblasts early and late after therapy administration (day 3 and 7) only at the higher dose, as measured by flow cytometry. FIG. 6C and FIG. 6D show the PD-L1 expression in cancer cells (FIG. 6C) and fibroblasts (FIG. 6D) at different time points of the study. PD-L1 expression is also statistically significantly increased on both cell types at the higher dose, but only 3 days after the treatment and not 7 days after treatment.

FIG. 7 shows results of the histological analysis by immunofluorescence (3DIP) and illustrates the CD8 T cell infiltration and MHCI expression in tumor sections. MHCI expression is increased 3 days and 7 days after P1AG3755 administration in a dose dependent manner, corroborating flow cytometry results. Increase in MHCI expression is local and not the same for the whole tumor. MHCI expression patterns correlate with CD8 T cell presence in the tumor core.

In FIG. 8 it is shown that CD8 infiltration into the tumor was not statistically significantly different from the Vehicle group, as measured by Flow Cytometry. This is to be expected, as CD8 T cell infiltration follows MHCI expression, which is highly localised. Through immunofluorescent imaging it becomes clear that CD8 T cells are more strongly present in MHC-I positive areas at Day 3 post therapy, as compared to Day 7 post therapy.

FIG. 9 relates to the cytokine analysis and shows that the T cell attractant chemokine CXCL9 is statistically significantly increased in Serum only 7 days post therapy injection (at 10 mg/kg), but not before, not at lower doses and not in tumor tissue.

FIG. 10A shows a schematic representation of the sterically masked IFNG (P1AG8692) as described in Example 6. The IFNG variant is inserted between the upper and lower hinge region of the IgG1 antibody. FIG. 10B shows a schematic representation of the unmasked control compound (P1AG8697), wherein the IFNG variant is fused via a linker (linker 4) to the Fab. FIG. 10C is a graph showing the activity of sterically masked IFNG variants measured on HEK-blue IFNG reporter cells as relative absorbance.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, bi- or multispecific antibodies, immunoconjugates, antibody fragments and scaffold antigen binding proteins.

As used herein, the term “antibody that specifically binds to a a tumor-associated antigen” or “moiety that specifically binds to a a tumor-associated antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. an IFNG variant polypeptide) to a target site, for example to a specific type of tumor cell or tumor stroma bearing the antigenic determinant. Antigen binding domains capable of specific binding to a tumor-associated antigen include antibodies and fragments thereof as further defined herein. In addition, antibodies capable of specific binding to a tumor-associated antigen include scaffold antigen binding proteins as further defined herein, e.g. binding domains which are based on designed repeat proteins or designed repeat domains (see e.g. WO 2002/020565). In particular, the antibody capable of specific binding to a tumor-associated antigen is an antibody capable of specific binding to Fibroblast Activation Protein (FAP). In relation to an antibody or fragment thereof, the term “antibody that specifically binds to a a tumor-associated antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a tumor-associated antigen” can also be a Fab fragment or a cross-Fab fragment.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells. A bispecific antigen binding molecule as described herein can also form part of a multispecific antibody.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region. According to the present invention, the term “Fab fragment” also includes “cross-Fab fragments” or “crossover Fab fragments” as defined below.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482:337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23 (12), 1556-1561 (2005) and Expert Opinion on Investigational Drugs 16 (6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100 (4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include up to 25 amino acids without affecting the overall fold of the microprotein. For further details of engineered knottin domains, see WO2008098796.

An “antibody that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more. An “antibody that does not bind to the same epitope” as a reference molecule refers to an antigen binding molecule that does not block binding of the reference molecule to its antigen in a competition assay by 50% or more, and conversely, the reference molecule does not block binding of the antigen binding molecule to its antigen in a competition assay by 50% or more.

The term “antigen binding domain” or “antigen-binding site” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤ 0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

A “tumor-associated antigen” or TAA as used herein refers to an antigenic determinant presented on the surface of a target cell, for example a cell in a tumor such as a cancer cell or a cell of the tumor stroma. In certain embodiments, the target cell antigen is an antigen on the surface of a tumor cell. In one embodiment, TAA is selected from the group consisting of Fibroblast Activation Protein (FAP), Carcinoembryonic Antigen (CEA), Folate receptor alpha (FolR1), Melanoma-associated Chondroitin Sulfate Proteoglycan (MCSP), Epidermal Growth Factor Receptor (EGFR), human epidermal growth factor receptor 2 (HER2) and p95HER2. In particular, the tumor-associated antigen is Fibroblast Activation Protein (FAP).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:46), or NCBI (www.ncbi.nlm.nih.gov/) RefSeq NP_004451.2. The extracellular domain (ECD) of human FAP extends from amino acid position 26 to 760. The amino acid sequence of mouse FAP is shown in UniProt accession no. P97321 (version 126, SEQ ID NO:47), or NCBI RefSeq NP 032012.1. The extracellular domain (ECD) of mouse FAP extends from amino acid position 26 to 761. Preferably, an anti-FAP binding molecule of the invention binds to the extracellular domain of FAP.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG1, IgG2, IgG3, IgG4, IgA1, and IgA2. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO: 48). Usually, a segment having the amino acid sequence of EPKSC (SEQ ID NO:49) is following to link the CH1 domain to the hinge region.

The term “hinge region” denotes the part of an antibody heavy chain polypeptide that joins in a wild-type antibody heavy chain the CH1 domain and the CH2 domain, e.g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083). In one aspect, the hinge region has the amino acid sequence DKTHTCPXCP (SEQ ID NO: 50), wherein X is either S or P. In one aspect, the hinge region has the amino acid sequence HTCPXCP (SEQ ID NO: 51), wherein X is either S or P. In one aspect, the hinge region has the amino acid sequence CPXCP (SEQ ID NO: 52), wherein X is either S or P.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. (EU numbering system according to Kabat). In one aspect, a CH2 domain has the amino acid sequence of APELLGGPSV FLFPPKPKDT LMISRTPEVT CVWDVSHEDP EVKFNWYVDG VEVHNAKTKP REEQESTYRW SVLTVLHQDW LNGKEYKCKV SNKALPAPIE KTISKAK (SEQ ID NO: 53). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 according to EU numbering system according to Kabat of an IgG). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO: 54). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “wild-type Fc domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 55 (IgG1, caucasian allotype), SEQ ID NO: 56 (IgG1, afroamerican allotype), SEQ ID NO: 57 (IgG2), SEQ ID NO: 58 (IgG3) and SEQ ID NO:59 (IgG4). The term “variant (human) Fc domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector function” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492, Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “IFNG”, as used herein, refers to any interferon gamma polypeptide, including a recombinantly produced polypeptide, a synthetically produced polypeptide, and IFNG isolated from cells or tissues, such as from T-lymphocytes and NK cells and other sources. As isolated from any source or as produced, IFNG polypeptides can be heterogeneous in length and typically range from 124 to 146 amino acids in length. Heterogeneity is typically observed at both termini, at the N-terminus due to post-translational removal of Cys-Tyr-Cys amino acids and at the C-terminus due to variable proteolytic processing. Heterogeneity also can result due to N-glycosylation of the polypeptide. Heterogeneity of IFNG polypeptides can differ depending on the source of the IFNG polypeptide. Hence reference to IFNG polypeptides refers to the heterogeneous population as produced or isolated.

IFNG is a cytokine that is secreted in response to viral infections or cancerous growths. IFNG regulates T-cell class I and II MHC antigen expression, Fc receptors, and macrophages. IFNG signals through a multimeric receptor complex consisting of two different chains: the IFNG receptor binding subunit (IFNGR, IFNGR1), and a transmembrane accessory factor (IFNGR2). The IFNG signaling complex is formed upon ligand-driven dimerization of the IFNG receptors. The term “IFNG polypeptide” as used herein may include its monomeric or dimeric form, as appropriate. The term “human IFNG” (huIFNG) as used herein includes IFNG, allelic variant isoforms, synthetic molecules, proteins isolated from human tissue and cells, and modified forms thereof, an exemplary human wild-type IFNG polypeptide is shown in SEQ ID NO: 60. The term “mouse IFNG” (muIFNG) as used herein includes IFNG, allelic variant isoforms, synthetic molecules, proteins isolated from murine tissue and cells, and modified forms thereof, an exemplary mouse wild-type IFNG polypeptide is shown in SEQ ID NO: 61.

As used herein, the term “IFNG variant polypeptide” refers to a monomeric IFNG polypeptide in which one or more amino acids are truncated and/or in which amino acid substitutions, deletions or insertions are present as compared to the amino acid sequence of a wild-type IFNG polypeptide.

The terms “anti-IFNG antibody”, “anti-IFNG”, “IFNG antibody and “an antibody that specifically binds to IFNG” refer to an antibody or antibody fragment that specifically binds to IFNG with sufficient affinity such that the antibody or antibody fragment is useful as a diagnostic and/or therapeutic agent in targeting IFNG. When associated with a molecule that comprises IFNG, the anti-IFNG antibody can function as a masking moiety of the molecule. Specifically disclosed herein are scFvs specific for IFNG that can block the activity of the cytokine. In one aspect, the extent of binding of an anti-IFNG antibody to an unrelated, non-IFNG protein is less than about 10% of the binding of the antibody to IFNG as measured, e.g., by a radioimmunoassay (RIA) or flow cytometry (FACS). In certain embodiments, an antibody that binds to IFNG has a dissociation constant (K_{D}) of ≤1 μM, ≤100 nM, ≤10 nM, ≤1 nM, ≤ 0.1 nM, ≤0.01 nM, or ≤0.001 nM (e.g. 10⁻⁶ M or less, e.g. from 10⁻⁶⁸ M to 10⁻¹³ M, e.g., from 10⁻⁸ M to 10⁻¹⁰ M). In one aspect, the anti-IFNG antibody is an antibody fragment, in particular an scFv.

The term “linker” or “peptide linker” refers to a peptide comprising one or more amino acids, typically about 2 to 30 amino acids. Peptide linkers are known in the art or are described herein. Suitable, non-immunogenic linker peptides are, for example, (G4S) n, (SG4) n or G4 (SG4) n peptide linkers, wherein “n” is generally a number between 1 and 10, typically between 2 and 5, in particular 3, i.e. the peptides selected from the group consisting of GGGGS (SEQ ID NO:62), GGGGSGGGGS (SEQ ID NO:63), SGGGGSGGGG (SEQ ID NO:64), GGGGSGGGGSGGGG (SEQ ID NO:65), (G4S) 3 (SEQ ID NO:66), (G4S) 4 (SEQ ID NO:67) and (G4S) 5 (SEQ ID NO:68), GGGGSGGGGSGGGGGGGGSGGGGSGGGGG (SEQ ID NO: 69), and GGGGSGGG (SEQ ID NO:72), but also included are peptide linkers such as LEVLFQG (SEQ ID NO:73) or GGGGSGGGGSGGGGSGGGGSGLEVLFQGPGGGGGSGGGGG (SEQ ID NO:74). Peptide linkers of particular interest are the protease cleavable linkers (“cleavable linkers”) GGGGSGGGGSGGGPQARKGGGGGGSGGGGG (SEQ ID NO:70) or GGGGSGGGGSGGGHQARKGGGGGGSGGGGG (SEQ ID NO:71). A “non-cleavable linker” is a peptide linker that will not be recognized by proteases and is stable against proteolysis.

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a heavy chain of an antibody and a Fab fragment) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611. Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997), Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

In certain embodiments, amino acid sequence variants of the bispecific antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include bispecific antigen binding molecules of the invention with an N-terminal methionyl residue. Other insertional variants of the molecule include the fusion to the N- or C-terminus to a polypeptide which increases the serum half-life of the bispecific antigen binding molecules.

In certain aspects, the bispecific antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of bispecific antigen binding molecules or antibodies of the invention are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). In another aspect, variants of the bispecific antigen binding molecules or antibodies of the invention are provided with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antigen binding molecules of the invention, e.g., “thioMAbs,” in which one or more residues of the molecule are substituted with cysteine residues. In particular aspects, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain aspects, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

“Protease activatable” as used herein, with respect to the antigen binding molecules comprising an interferon gamma (IFNG) variant, refers to a molecule comprising a IFNG variant with a reduced or abrogated ability to bind the IFNG receptors due to a masking moiety that reduces or abrogates the ability of the IFNG variant to bind to the IFNG receptor. Upon dissociation of the masking moiety by proteolytic cleavage, e.g., by proteolytic cleavage of a linker connecting the masking moiety to the molecule comprising a IFNG variant, binding to the IFNG receptor is restored and the IFNG variant is thereby able to activate.

“Reversibly concealing” as used herein refers to the binding of a masking moiety to an IFNG variant polypeptide such as to prevent the IFNG variant polypeptide from binding to its receptor. This concealing is reversible in that the masking moiety can be released from the IFNG variant polypeptide, e.g. by protease cleavage, and thereby freeing the IFNG variant polypeptide to bind to its receptor.

“Protease” or “proteolytic enzyme” as used herein refers to any proteolytic enzyme that cleaves the linker at a recognition site and that is expressed by a target cell. Such proteases might be secreted by the target cell or remain associated with the target cell, e.g., on the target cell surface. Examples of proteases include but are not limited to metalloproteinases, e.g., matrix metalloproteinase 1-28 and A Disintegrin And Metalloproteinase (ADAM) 2, 7-12, 15, 17-23, 28-30 and 33, serine proteases, e.g., urokinase-type plasminogen activator and Matriptase, cysteine proteases (e.g. cathepsin S) and matrix metalloproteinases (e.g. MMP-2 and MMP-9). Matriptase, matrix metalloproteinase 2 (MMP-2, gelatinase A) and matrix metalloproteinase 9 (MMP-9, gelatinase B) are overexpressed e.g. in breast- and ovarian carcinoma. MMP-2 and MMP-9 activity was detected in cervical, breast and ovarian carcinoma and ascites of patients with epithelial ovarian cancer (EOC) but not in the serum of these patients (Demeter, A. et al., Anticancer Res. 2005, 25, 2885-2889). While matriptase can be detected in normal epithelial cells, matriptase activity is mainly detected in cancer (LeBeau, A. M. et al., Proc. Natl. Acad. Sci. USA 2013, 110, 93-98).

The term “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a bispecific antigen binding molecule or antibody” refers to one or more nucleic acid molecules encoding the heavy and light chains (or fragments thereof) of the bispecific antigen binding molecule or antibody, including such nucleic acid molecule(s) in a single vector or separate vectors, and such nucleic acid molecule(s) present at one or more locations in a host cell.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

The term “chemotherapeutic agent” as used herein refers to a chemical compound useful in the treatment of cancer. In one aspect, the chemotherapeutic agent is an antimetabolite. In one aspect, the antimetabolite is selected from the group consisting of Aminopterin, Methotrexate, Pemetrexed, Raltitrexed, Cladribine, Clofarabine, Fludarabine, Mercaptopurine, Pentostatin, Thioguanine, Capecitabine, Cytarabine, Fluorouracil, Floxuridine, and Gemcitabine. In one particular aspect, the antimetabolite is capecitabine or gemcitabine. In another aspect, the antimetabolite is fluorouracil. In one aspect, the chemotherapeutic agent is an agent that affects microtubule formation. In one aspect, the agent that affects microtubule formation is selected from the group consisting of: paclitaxel, docetaxel, vincristine, vinblastine, vindesine, vinorelbin, taxotere, etoposide, and teniposide. In another aspect, the chemotherapeutic agent is an alkylating agent such as cyclophosphamide. In one aspect, the chemotherapeutic agent is a cytotoxic antibiotic such as a topoisomerase II inhibitor. In one aspect, the topoisomerase II inhibitor is doxorubicin.

The term “agent for use in cancer immunotherapy” refers to any substance including a monoclonal antibody that effects the immune system. The antigen binding molecules described herein can be considered as such agents. Agents for use in cancer immunotherapy can be used as anti-neoplastic agents for the treatment of cancer. In one aspect, said agents include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), LAG3 antibodies (e.g. relatlimab), PD1-LAG3 bispecific antibodies and TIGIT antibodies (e.g. tiragolumab).

### Antigen Binding Molecules Containing an Interferon Gamma (IFNG) Variant Polypeptide

The invention provides new antigen binding molecules containing an interferon gamma (IFNG) variant polypeptide that are capable of specific binding to a tumor-associated antigen, in particular Fibroblast activation protein (FAP) and thus combine an antibody that specifically binds to FAP with a new IFNG variant polypeptide homodimer. The antigen binding molecules described herein possess particularly advantageous properties such as producibility, stability, binding affinity, biological activity, targeting efficiency, reduced sink effect, superior pharmacokinetic (PK) properties, reduced toxicity, an extended therapeutic window and thereby a possibly enhanced efficacy.

### Exemplary Antigen Binding Molecules

In one aspect, disclosed herein are antigen binding molecules that are characterized by comprising a homodimer of an interferon gamma (IFNG) variant polypeptide, wherein the IFNG variant polypeptide is characterized by the C-terminal amino acid sequence KRKRP (SEQ ID NO:1). It has been shown herein that the presence of the KRKR patch, i.e. the amino acid sequence of KRKR (SEQ ID NO:78) is important for the activity of IFNG. Recently, it has been confirmed that the KRKR patch is relevant for the binding of IFNG to the heparan sulfate moiety of the extracellular matrix and thereby prevents fatal systemic toxicity (J. Kemna et al, Nature Immunology 2023, 24, 414-422). In order to avoid proteolysis, the C-terminus of wild-type IFNG has been stabilized with a proline cap so that the sequence of the IFNG variant polypeptide terminates with the amino acid sequence KRKRP (SEQ ID NO:1).

Furthermore, the antigen binding molecules comprise a targeting moiety against a tumor-associated antigen, in particular FAP. In addition, the antigen binding molecules described herein comprise a Fc region which comprises mutations that reduce effector function. The use of an Fc region comprising mutations that reduce or abolish effector function will prevent unspecific agonism by crosslinking via Fc receptors and will prevent ADCC of FAP expressing cells. The antigen binding molecules as described herein possess the advantage over conventional IFNG in that they selectively induce immune response at the target cells, which are typically in the tumor stroma, i.e in proximity to the tumor.

In the presence of FAP-expressing cells the antigen binding molecules described herein are able to upregulate of MHCI and PDL1 expression (Example 5). Following the upregulation of MHCI expression, the antigen binding molecules are to increase the amount of CD8 T cells in the tumor tissue (Example 5). Furthermore, treatment with antigen binding molecules described herein leads to an increased CXCL9 (T cell attractant chemokine) level post therapy which is critical to the immune infiltration cascade.

Provided herein are antigen binding molecules, comprising
(i) an antibody that specifically binds to a tumor-associated antigen and
(ii) a homodimer of an interferon gamma (IFNG) variant polypeptide, wherein the IFNG variant polypeptide is characterized by the C-terminal amino acid sequence KRKRP (SEQ ID NO: 1).

Thus, provided are antigen binding molecules, comprising (i) an antibody that specifically binds to a tumor-associated antigen and (ii) a homodimer of an interferon gamma (IFNG) variant polypeptide, wherein the IFNG variant polypeptide is characterized in that it terminates at the C-terminus with the amino acid sequence KRKRP (SEQ ID NO:1).

The IFNG variant polypeptide is truncated at the C-terminus and protected with a proline at the C-terminus to protect it from proteolysis.

In one aspect, the antibody that specifically binds to a tumor-associated antigen is an antibody that specifically binds to Fibroblast activation protein (FAP). In one aspect, provided is an antigen binding molecule, wherein the antibody that specifically binds to FAP comprises (a) a heavy chain variable region (V_{H}FAP) comprising a heavy chain complementary determining region (CDR-H1) comprising the amino acid sequence of SEQ ID NO:4, a CDR-H2 comprising the amino acid sequence of SEQ ID NO:5, and a CDR-H3 comprising the amino acid sequence of SEQ ID NO:6, and a light chain variable region (V_{L}FAP) comprising a light chain complementarity determining region (iv) CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9, or (b) a heavy chain variable region (V_{H}FAP) comprising a CDR-H1 comprising the amino acid sequence of SEQ ID NO:12, a CDR-H2 comprising the amino acid sequence of SEQ ID NO:13, and a CDR-H3 comprising the amino acid sequence of SEQ ID NO: 14, and a light chain variable region (V_{L}FAP) comprising a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO: 17. In one aspect, provided is an antigen binding molecule, wherein the antibody that specifically binds to FAP comprises a heavy chain variable region (V_{H}FAP) comprising the amino acid sequence of SEQ ID NO:10 and a light chain variable region (V_{L}FAP) comprising the amino acid sequence of SEQ ID NO:11 or it comprises a heavy chain variable region (V_{H}FAP) comprising the amino acid sequence of SEQ ID NO:18 and a light chain variable region (V_{L}FAP) comprising the amino acid sequence of SEQ ID NO: 19. In one particular aspect, provided is an antigen binding molecule, wherein the antibody that specifically binds to FAP comprises a heavy chain variable region (V_{H}FAP) comprising the amino acid sequence of SEQ ID NO: 10 and a light chain variable region (V_{L}FAP) comprising the amino acid sequence of SEQ ID NO:11.

In one aspect, the antigen binding molecule comprises an Fc domain, in particular and IgG1 Fc domain or an IgG4 Fc domain. In one aspect, the Fc domain comprises one or more amino acid substitution that reduces the binding affinity of the antibody to an Fc receptor and/or effector function. In one aspect, the Fc domain is of human IgG1 subclass with the amino acid mutations L234A, L235A and P329G (EU numbering according to Kabat EU index). In another aspect, the Fc domain is a murine Fc domain and comprises the amino acid mutations D265A and P329G (EU numbering according to Kabat EU index).

In one aspect, the antigen binding molecule is protease-activatable and comprises a protease recognition site and a masking moiety. The antigen binding molecule thus comprises (i) an antibody that specifically binds to a tumor-associated antigen, (ii) a homodimer of an interferon gamma (IFNG) variant polypeptide, wherein the IFNG variant polypeptide is characterized in that it terminates at the C-terminus with the amino acid sequence KRKRP (SEQ ID NO:1), (iii) a protease recognition site and (iv) a masking moiety.

### Protease Recognition Site

In one aspect, the protease recognition site is a substrate for matriptase. In one aspect, the protease recognition site comprises or consists of the amino acid sequence PQARK (SEQ ID NO: 20) or HQARK (SEQ ID NO:21). In one particular aspect, the protease recognition site comprises or consists of the amino acid sequence PQARK (SEQ ID NO:20). In one aspect, the protease recognition site is part of a cleavable peptide linker which connects the masking moiety with the IFNG variant polypeptide. In one aspect, the cleavable peptide linker comprises the amino acid sequence of SEQ ID NO:70 or SEQ ID NO:71, in a particular aspect the cleavable peptide linker comprises the amino acid sequence of SEQ ID NO:70.

In one aspect, provided is an antigen binding molecule, wherein the masking moiety is fused at its N-terminus to the C-terminus of the IFNG variant polypeptide via the cleavable peptide linker (mask release format). In one aspect, the IFNG variant polypeptide is fused at its N-terminus via a stable linker to the C-terminus of the antibody.

In another aspect, provided is an antigen binding molecule, wherein the masking moiety is fused at its N-terminus to the C-terminus of the Fc domain via a stable linker and at its C-terminus to the N-terminus of the IFNG variant polypeptide via the cleavable peptide linker (cytokine release format).

### Masking Moiety

The protease-activatable antigen binding molecule comprises a masking moiety, in particular two masking moieties for the IFNG variant homodimer. In one aspect, the masking moiety is an antibody fragment that specifically binds to the IFNG variant polypeptide and is able to mask it. In one particular aspect, the masking moiety is an scFv that specifically binds to the IFNG variant polypeptide.

In one aspect, the masking moiety that specifically binds to the IFNG variant polypeptide, in particular an scFv, comprises
(a) a heavy chain variable region (V_{H}IFNG) comprising a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a light chain variable region (V_{L}IFNG) comprising a light chain complementarity determining region (iv) CDR-L1 comprising the amino acid sequence of SEQ ID NO:25, a CDR-L2 comprising the amino acid sequence of SEQ ID NO:26, and a CDR-L3 comprising the amino acid sequence of SEQ ID NO:27, or
(b) a heavy chain variable region (V_{H}IFNG) comprising a CDR-H1 comprising the amino acid sequence of SEQ ID NO:30, a CDR-H2 comprising the amino acid sequence of SEQ ID NO:31, and a CDR-H3 comprising the amino acid sequence of SEQ ID NO:32, and a light chain variable region (V_{L}IFNG) comprising a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35.

In one aspect, the masking moiety that specifically binds to the IFNG variant polypeptide, in particular an scFv, comprises (a) a heavy chain variable region (V_{H} IFNG) comprising the amino acid sequence of SEQ ID NO:28 and a light chain variable region (V_{L} IFNG) comprising the amino acid sequence of SEQ ID NO:29, or (b) a heavy chain variable region (V_{H} IFNG) comprising the amino acid sequence of SEQ ID NO:36 and a light chain variable region (V_{L} IFNG) comprising the amino acid sequence of SEQ ID NO:37. In one particular aspect, the masking moiety that specifically binds to the IFNG variant polypeptide, in particular an scFv, comprises (a) a heavy chain variable region (V_{H} IFNG) comprising the amino acid sequence of SEQ ID NO:28 and a light chain variable region (V_{L} IFNG) comprising the amino acid sequence of SEQ ID NO:29.

In one aspect, the masking moiety that specifically binds to the IFNG variant polypeptide and masks the IFNG variant polypeptide comprises an amino acid sequence that is at least about 95%, 96%, 97%, 98%, 99% or 100% identical to SEQ ID NO: 38. In one aspect, the masking moiety that specifically binds to the IFNG variant polypeptide comprises the amino acid sequence of SEQ ID NO: 38.

In another aspect, the masking moiety that specifically binds to the IFNG variant polypeptide and masks the IFNG variant polypeptide comprises an amino acid sequence that is at least about 95%, 96%, 97%, 98%, 99% or 100% identical to SEQ ID NO: 39. In one aspect, the masking moiety that specifically binds to the IFNG variant polypeptide comprises the amino acid sequence of SEQ ID NO: 39.

### Mask Release Format and Cytokine Release Format

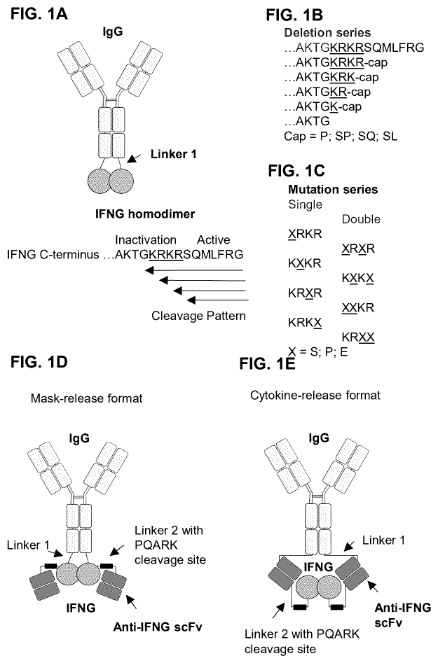

In one aspect, provided is an antigen binding molecule, wherein the masking moiety is fused at its N-terminus to the C-terminus of the IFNG variant polypeptide via the cleavable peptide linker (mask release format). In one aspect, the IFNG variant polypeptide is fused at its N-terminus via a stable linker to the C-terminus of the antibody. A schematic scheme of the format is shown in FIG. 1D.

In one particular aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:40 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:40 and two light chains comprising the amino acid sequence of SEQ ID NO:41.

In another particular aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:43 and two light chains comprising an amino acid sequence that is at least about 95%, 96%, 97%, 98%, 99% or 100% identical to SEQ ID NO:44. In one aspect, the antigen binding molecule comprises two heavy chains comprising the amino acid sequence of SEQ ID NO:43 and two light chains comprising the amino acid sequence of SEQ ID NO:44.

In another aspect, provided is an antigen binding molecule, wherein the masking moiety is fused at its N-terminus to the C-terminus of the Fc domain via a stable linker and at its C-terminus to the N-terminus of the IFNG variant polypeptide via the cleavable peptide linker (cytokine release format). A schematic scheme of the format is shown in FIG. 1E.

In one particular aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:42 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:42 and two light chains comprising the amino acid sequence of SEQ ID NO:41.

In another particular aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:45 and two light chains comprising an amino acid sequence that is at least about 95%, 96%, 97%, 98%, 99% or 100% identical to SEQ ID NO:44. In one aspect, the antigen binding molecule comprises two heavy chains comprising the amino acid sequence of SEQ ID NO:45 and two light chains comprising the amino acid sequence of SEQ ID NO:44.

### Formats Comprising a Sterically Masked IFNG Variant Polypeptide

Provided herein are also antigen binding molecules, comprising
(i) an antibody that specifically binds to a tumor-associated antigen and
(ii) a homodimer of an interferon gamma (IFNG) variant polypeptide, wherein the IFNG variant polypeptide is characterized in that it terminates at the C-terminus with the amino acid sequence KRKRP (SEQ ID NO:1) and wherein the IFNG variant polypeptide is inserted between the upper and lower hinge region of the antibody.

These antigen binding molecules are characterized in that they comprise a sterically hidden, i.e a sterically masked IFNG variant polypeptide.

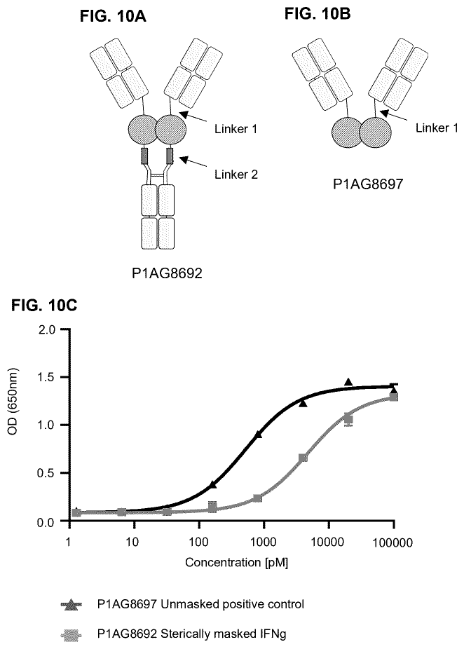

In one aspect, provided is an antigen binding molecule, wherein the IFNG variant polypeptide is fused at its N-terminus via a first linker to the C-terminus to the VHCH1 chain of the Fab domain that specifically binds to a tumor-associated antigen, in particular FAP. In addition, the IFNG variant polypeptide is fused at its C-terminus via a second linker to the N-terminus to the lower hinge region of the Fc domain. A schematic scheme of the format is shown in FIG. 10A.

In one aspect, the first linker is glycine-serine linker. In one particular aspect, the first linker has the amino acid sequence of SEQ ID NO:69.

In one aspect, the second linker is glycine-serine linker or a cleavable linker. In one particular aspect, the second linker has an amino acid sequence selected from the group consisting of SEQ ID NO:72, SEQ ID NO:73 or SEQ ID NO:75.

In one aspect, the lower hinge region has the amino acid sequence of SEQ ID NO:76. In another aspect, the lower hinge region is shortened and has the amino acid sequence of SEQ ID NO: 77.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:119 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:119 and two light chains comprising the amino acid sequence of SEQ ID NO:41.

In another aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:120 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:120 and two light chains comprising the amino acid sequence of SEQ ID NO:41.

In another aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:121 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:121 and two light chains comprising the amino acid sequence of SEQ ID NO:41.

In yet another aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:122 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:122 and two light chains comprising the amino acid sequence of SEQ ID NO:41.

In another aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:123 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:124 and two light chains comprising the amino acid sequence of SEQ ID NO:41.

Fc Domain Modifications Reducing Fc Receptor Binding and/or Effector Function

The antigen binding molecules of the invention further comprise a Fc domain composed of a first and a second subunit and comprising one or more amino acid substitution that reduces the binding affinity of the antigen binding molecule to an Fc receptor and/or effector function. Thus, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular embodiments the Fc domain of the bispecific antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 Fc domain.

In one such aspect the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human FcγRIIB. In one aspect the effector function is one or more of CDC, ADCC, ADCP, and cytokine secretion. In a particular aspect, the effector function is ADCC. In one aspect, the Fc domain domain exhibits substantially similar binding affinity to neonatal Fc receptor (FcRn), as compared to a native IgG1 Fc domain. Substantially similar binding to FcRn is achieved when the Fc domain (or the the bispecific antigen binding molecule of the invention comprising said Fc domain) exhibits greater than about 70%, particularly greater than about 80%, more particularly greater than about 90% of the binding affinity of a native IgG1 Fc domain (or the the bispecific antigen binding molecule of the invention comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific antigen binding molecule of the invention comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor is an inhibitory Fc receptor. In a specific aspect, the Fc receptor is an inhibitory human Fcγ receptor, more specifically human FcγRIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the bispecific antigen binding molecule of the invention comprising said Fc domain) exhibits greater than about 70% of the binding affinity of a non-engineered form of the Fc domain (or the bispecific antigen binding molecule of the invention comprising said non-engineered form of the Fc domain) to FcRn. The Fc domain, or the the bispecific antigen binding molecule of the invention comprising said Fc domain, may exhibit greater than about 80% and even greater than about 90% of such affinity. In certain embodiments the Fc domain of the bispecific antigen binding molecule of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at a position of E233, L234, L235, N297, P331 and P329. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331S. In more preferred aspects, the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions. Such antibody is an IgG1 with mutations L234A and L235A or with mutations L234A, L235A and P329G (numbering according to EU index of Kabat et al, Kabat et al., Sequences of Proteins of Immunological Interest, 5th Ed. Public Health Service, National Institutes of Health, Bethesda, MD, 1991).

In one aspect, the Fc domain is an IgG4 Fc domain. In a more specific embodiment, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)).

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antigen binding molecules of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

The following section describes preferred aspects of the antigen binding molecules described herein comprising Fc domain modifications reducing Fc receptor binding and/or effector function. In one aspect, provided is an antigen binding molecule comprising (i) an antibody that specifically binds to a tumor associated antigen and (ii) a homodimer of an interferon gamma (IFNG) variant polypeptide, wherein the IFNG variant polypeptide is characterized by the C-terminal amino acid sequence KRKRP (SEQ ID NO:1) and wherein the antigen binding molecule comprises an IgG1 Fc domain or an IgG4 Fc domain and wherein the Fc domain comprises one or more amino acid substitution that reduces the binding affinity of the antibody to an Fc receptor and/or effector function. In another aspect, provided is an antigen binding molecule comprising (i) an antibody that specifically binds to a tumor associated antigen and (ii) a homodimer of an interferon gamma (IFNG) variant polypeptide, wherein the IFNG variant polypeptide is characterized in that it terminates at the C-terminus with the amino acid sequence KRKRP (SEQ ID NO:1) and wherein the Fc domain is of human IgG1 subclass with the amino acid mutations L234A, L235A and P329G (EU numbering according to Kabat EU index).

### Exemplary IFNG Variant Polypeptides

In one aspect, provided is an IFNG variant polypeptide that is characterized in that it terminates at the C-terminus with the 1 amino acid sequence KRKRP (SEQ ID NO:1). It has been shown herein that the presence of the KRKR patch, i.e. the amino acid sequence of KRKR (SEQ ID NO:78) is important for the activity of IFNG. In order to avoid proteolysis, the C-terminus of wild-type IFNG has been stabilized with a proline cap so that the sequence of the IFNG variant polypeptide terminates with the amino acid sequence KRKRP (SEQ ID NO:1).

In one aspect, provided is an IFNG variant polypeptide that comprises or consists of the amino acid sequence of SEQ ID NO:2. In another aspect, provided is an IFNG variant polypeptide that comprises or consists of the amino acid sequence of SEQ ID NO:3.

Provided herein are also mutation or insertion variants of the IFNG variant polypeptide that is characterized in that it terminates at the C-terminus with the amino acid sequence KRKRP (SEQ ID NO:1).

In one aspect, provided herein is an IFNG variant polypeptide that is characterized in that it terminates at the C-terminus with the amino acid sequence KRKRP (SEQ ID NO:1), wherein one or two of amino acids in SEQ ID NO: 1 are replaced by an amino acid selected from the group consisting of serine(S), proline (P) and glutamate (E). Thus provided herein is an IFNG variant polypeptide that is characterized in that it terminates at the C-terminus with the an amino acid sequence selected from the group consisting of AKTGSRKRP (SEQ ID NO: 137), AKTGKSKRP (SEQ ID NO:138), AKTGKRSRP (SEQ ID NO:139), AKTGKRKSP (SEQ ID NO:134), AKTGPRKRP (SEQ ID NO:140), AKTGKPKRP (SEQ ID NO:141), AKTGKRPRP (SEQ ID NO:142), AKTGKRPPP ((SEQ ID NO: 143), AKTGERKRP (SEQ ID NO: 144), AKTGKEKRP (SEQ ID NO: 145), AKTGKRERP (SEQ ID NO: 146), AKTGKRKEP (SEQ ID NO:147), AKTGSRSRP (SEQ ID NO:148), AKTGPRPRP (SEQ ID NO:149), AKTGERERP (SEQ ID NO:150), AKTGKSKSP (SEQ ID NO:151), AKTGKPKPP (SEQ ID NO: 152), AKTGKEKEP (SEQ ID NO: 153), AKTGSSKRP (SEQ ID NO:154), AKTGPPKRP (SEQ ID NO:155), AKTGEEKRP (SEQ ID NO:156), AKTGKRSSP (SEQ ID NO:157), AKTGKRPPP (SEQ ID NO:158) and AKTGKREEP (SEQ ID NO:159).

In one preferred aspect, provided herein is an IFNG variant polypeptide that is characterized in that it terminates at the C-terminus with the amino acid sequence KRKRP (SEQ ID NO:1), wherein one or two of amino acids in SEQ ID NO:1 are replaced by an amino acid selected from serine(S) or proline (P). This includes IFNG variant polypeptides that are characterized in that they terminate at the C-terminus with an amino acid sequence selected from the group consisting of AKTGSRKRP (SEQ ID NO:137), AKTGKSKRP (SEQ ID NO: 138), AKTGKRSRP (SEQ ID NO:139), AKTGKRKSP (SEQ ID NO: 134), AKTGPRKRP (SEQ ID NO:140), AKTGKPKRP (SEQ ID NO:141), AKTGKRPRP (SEQ ID NO:142), AKTGKRPPP ((SEQ ID NO: 143), AKTGSRSRP (SEQ ID NO: 148), AKTGPRPRP (SEQ ID NO: 149), AKTGKSKSP (SEQ ID NO:151), AKTGKPKPP (SEQ ID NO:152), AKTGSSKRP (SEQ ID NO:154), AKTGPPKRP (SEQ ID NO:155), AKTGKRSSP (SEQ ID NO:157) and AKTGKRPPP (SEQ ID NO:158).

Provided herein are also antigen binding molecules, comprising
(i) an antibody that specifically binds to a tumor-associated antigen and
(ii) a homodimer of an interferon gamma (IFNG) variant polypeptide, wherein the IFNG variant polypeptide is characterized by in that it terminates at the C-terminus with the amino acid sequence KRKRP (SEQ ID NO:1) and wherein one or two of amino acids in SEQ ID NO: 1 are replaced by an amino acid selected from the group consisting of serine(S), proline (P) and glutamate (E).

In one particular aspect, provided are antigen binding molecules comprising
(i) an antibody that specifically binds to a tumor-associated antigen and
(ii) a homodimer of an interferon gamma (IFNG) variant polypeptide, wherein the IFNG variant polypeptide is characterized in that it terminates at the C-terminus with the amino acid sequence KRKRP (SEQ ID NO:1) and wherein one or two of amino acids in SEQ ID NO:1 are replaced by an amino acid selected from serine(S) or proline (P).

In one aspect, provided are antigen binding molecules comprising
(i) an antibody that specifically binds to a tumor-associated antigen and
(ii) a homodimer of an interferon gamma (IFNG) variant polypeptide, wherein the IFNG variant polypeptide is characterized by the C-terminal amino acid sequence selected from the group consisting of AKTGSRKRP (SEQ ID NO:137), AKTGKSKRP (SEQ ID NO:138), AKTGKRSRP (SEQ ID NO: 139), AKTGKRKSP (SEQ ID NO:134), AKTGPRKRP (SEQ ID NO: 140), AKTGKPKRP (SEQ ID NO:141), AKTGKRPRP (SEQ ID NO: 142), AKTGKRPPP ((SEQ ID NO:143), AKTGSRSRP (SEQ ID NO:148), AKTGPRPRP (SEQ ID NO:149), AKTGKSKSP (SEQ ID NO:151), AKTGKPKPP (SEQ ID NO:152), AKTGSSKRP (SEQ ID NO: 154), AKTGPPKRP (SEQ ID NO:155), AKTGKRSSP (SEQ ID NO:157) and AKTGKRPPP (SEQ ID NO:158).

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:91 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:91 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:92 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:92 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:93 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:93 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:94 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:94 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:95 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:95 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:96 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:96 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:97 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:97 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:98 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:98 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:103 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:103 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:104 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:104 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:106 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:106 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:107 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:107 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:109 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:109 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:110 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:110 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:112 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:112 and two light chains comprising the amino acid sequence of SEQ ID NO:41.

In one aspect, provided herein is an antigen binding molecule containing an IFNG variant polypeptide, wherein said antigen binding molecule comprises two heavy chains comprising an amino acid sequence that is at least about 95%, 96%, 97%, 98%, 99% or 100% identical to SEQ ID NO:113 and two light chains comprising an amino acid sequence that is at least about 95%, 96%, 97%, 98%, 99% or 100% identical to SEQ ID NO:41. In one aspect, the antigen binding molecule comprises two heavy chains comprising the amino acid sequence of SEQ ID NO:113 and two light chains comprising the amino acid sequence of SEQ ID NO:41.

### Polynucleotides

Provided herein are also isolated polynucleotides encoding an antigen binding molecule as described herein or a fragment thereof or isolated polynucleotides encoding an IFNG variant polypeptide as described herein.

The isolated polynucleotides encoding the antigen binding molecules disclosed herein may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes a polypeptide comprised in the antigen binding molecule as described herein. In one aspect, provided is an isolated polynucleotide encoding an IFNG variant polypeptide, wherein the IFNG variant polypeptide is characterized by the C-terminal amino acid sequence KRKRP (SEQ ID NO:1)

In one aspect, provided is an isolated polynucleotide encoding an antigen binding molecule, comprising (i) an antibody that specifically binds to a tumor associated antigen and (ii) a homodimer of an IFNG variant polypeptide, wherein the IFNG variant polypeptide is characterized by the C-terminal amino acid sequence KRKRP (SEQ ID NO:1). In one aspect, provided is one or more isolated polynucleotide encoding an antigen binding molecule, comprising (i) an antibody that specifically binds to a tumor associated antigen and (ii) a homodimer of an IFNG variant polypeptide, wherein the IFNG variant polypeptide is characterized by the C-terminal amino acid sequence KRKRP (SEQ ID NO:1), (iii) a protease recognition site and (iv) a masking moiety.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

Antigen binding molecules as described herein may be obtained, for example, by recombinant production. For recombinant production one or more polynucleotide encoding the antigen binding molecule or polypeptide fragments thereof are provided. The one or more polynucleotide encoding the bispecific antigen binding molecule are isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N. Y. (1989). The expression vector can be part of a plasmid, virus, or may be a nucleic acid fragment. The expression vector includes an expression cassette into which the polynucleotide encoding the antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antigen binding molecule disclosed herein or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide as provided herein. For example, if secretion of the antigen binding molecule or polypeptide fragments thereof is desired, DNA encoding a signal sequence may be placed upstream of the nucleic acid encoding the antigen binding molecule or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a bispecific antigen binding molecule of the invention or polypeptide fragments thereof.

In a further aspect, a host cell comprising one or more polynucleotides of the invention is provided. In certain aspects, a host cell comprising one or more vectors is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) the antigen binding molecules described herein. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing an antigen binding molecule as disclosed herein or polypeptide fragments thereof is provided, wherein the method comprises culturing a host cell comprising polynucleotides encoding the antigen binding molecule or polypeptide fragments thereof, as provided herein, under conditions suitable for expression of the antigen binding molecule or polypeptide fragments thereof, and recovering the antigen binding molecule of the invention or polypeptide fragments thereof from the host cell (or host cell culture medium).

Antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the examples. The purity of the antigen binding molecule or fragments thereof can be determined by any of a variety of well-known analytical methods including gel electrophoresis, high pressure liquid chromatography, and the like. For example, the antigen binding molecules expressed as described in the Examples were shown to be intact and properly assembled as demonstrated by reducing and non-reducing SDS-PAGE.

### Assays

The antigen binding molecules provided herein may be characterized for their binding properties and/or biological activity by various assays known in the art. In particular, they are characterized by the assays described in more detail in the examples.

### 1. Binding Assay

Binding of the antigen binding molecules and IFNG variant polypeptides provided herein to the corresponding target expressing cells may be evaluated for example by using a murine fibroblast cell line expressing human Fibroblast Activation Protein (FAP) and flow cytometry (FACS) analysis. The binding behaviour of IFNG variants towards IFNGR1 and IFNGR1/2 can be assessed by surface plasmon resonance (SPR) as described in the Examples.

### 2. Activity Assays

The antigen binding molecules and IFNG variant polypeptides as described herein are tested for biological activity. Biological activity may include efficacy and specificity of the bispecific antigen binding molecules. The activity of the IFNG variant polypeptides can be measured by the HEK Blue IFNG reporter cell assay as described in the Examples. Furthermore, the activity of the IFNG variant polypeptides and antigen binding molecules can be measured by the upregulation of MHC-I and PD-L1 on murine MC38-huCEA tumor cell lines and the upregulation of chemoattractants (CXCL9) as described in the Examples.

### Pharmaceutical Compositions, Formulations and Routes of Administration

In a further aspect, provided are pharmaceutical compositions comprising any of the antigen binding molecules provided herein, e.g., for use in any of the below therapeutic methods. In one aspect, a pharmaceutical composition comprises any of the antigen binding molecules provided herein and at least one pharmaceutically acceptable excipient. In another aspect, a pharmaceutical composition comprises any of the antigen binding molecules provided herein and at least one additional therapeutic agent, e.g., as described below.

Pharmaceutical compositions as described herein comprise a therapeutically effective amount of one or more antigen binding molecules dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one antigen binding molecule as disclosed herein and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intra-lesional, intravenous, intra-arterial, intramuscular, intrathecal or intraperitoneal injection. For injection, the antigen binding molecules disclosed herein may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the antigen binding molecules may be in powder form for constitution with a suitable vehicle, e.g., sterile pyrogen-free water, before use. Sterile injectable solutions are prepared by incorporating the antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular aspects, prolonged absorption of an injectable composition can be brought about by the use in the compositions of agents delaying absorption, such as, for example, aluminum monostearate, gelatin or combinations thereof.

In addition to the compositions described previously, the antigen binding molecules may also be formulated as a depot preparation. Such long acting formulations may be administered by implantation (for example subcutaneously or intramuscularly) or by intramuscular injection. Thus, for example, the antigen binding molecules may be formulated with suitable polymeric or hydrophobic materials (for example as emulsion in an acceptable oil) or ion exchange resins, or as sparingly soluble derivatives, for example, as a sparingly soluble salt.

Pharmaceutical compositions comprising the antigen binding molecules as described herein may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The antigen binding molecules as disclosed herein may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms. The composition herein may also contain more than one active ingredients as necessary for the particular indication being treated, preferably those with complementary activities that do not adversely affect each other. Such active ingredients are suitably present in combination in amounts that are effective for the purpose intended. The formulations to be used for in vivo administration are generally sterile. Sterility may be readily accomplished, e.g., by filtration through sterile filtration membranes.

### Therapeutic Methods and Compositions

Any of the antigen binding molecules provided herein may be used in therapeutic methods. For use in therapeutic methods, the antigen binding molecules as described herein can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, antigen binding molecules or IFNG variant polypeptides as disclosed herein for use as a medicament are provided.

In further aspects, antigen binding molecules or IFNG variant polypeptides as described herein for use in upregulating CXCL9 are provided. Furthermore, antigen binding molecules as described herein are provided for the (i) the treatment of cancer, (ii) delaying progression of cancer, and (iii) prolonging the survival of a patient suffering from cancer, in particular in the presence of FAP-expressing cells. In a particular aspect, the antigen binding molecules or IFNG variant polypeptides as disclosed herein for use in treating a disease, in particular for use in the treatment of cancer, are provided.

In certain aspects, the antigen binding molecules or IFNG variant polypeptides as described herein for use in a method of treatment are provided. In one aspect, provided is an antigen binding molecule or IFNG variant polypeptide as described herein for use in the treatment of a disease in an individual in need thereof. In certain aspects, provided is an antigen binding molecule or IFNG variant polypeptide for use in a method of treating an individual having a disease comprising administering to the individual a therapeutically effective amount of the bispecific antigen binding molecule. In certain aspects the disease to be treated is cancer. The subject, patient, or “individual” in need of treatment is typically a mammal, more specifically a human.

In a further aspect, the invention provides for the use of the antigen binding molecule or IFNG variant polypeptide described herein in the manufacture or preparation of a medicament for the treatment of a disease in an individual in need thereof. In one aspect, the medicament is for use in a method of treating a disease comprising administering to an individual having the disease a therapeutically effective amount of the medicament. In certain aspects, the disease to be treated is a proliferative disorder, particularly cancer. Examples of cancers include, but are not limited to, bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other examples of cancer include carcinoma, lymphoma (e.g., Hodgkin's and non-Hodgkin's lymphoma), blastoma, sarcoma, and leukemia. Other cell proliferation disorders that can be treated using the bispecific antigen binding molecule or antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the antigen binding molecule or IFNG variant polypeptide as described herein may not provide a cure but may provide a benefit. In some aspects, a physiological change having some benefit is also considered therapeutically beneficial. Thus, in some aspects, an amount of the antigen binding molecule or IFNG variant polypeptide that provides a physiological change is considered an “effective amount” or a “therapeutically effective amount”.

For the prevention or treatment of disease, the appropriate dosage of the antigen binding molecule described herein (when used alone or in combination with one or more other additional therapeutic agents) will depend on the type of disease to be treated, the route of administration, the body weight of the patient, the specific molecule, the severity and course of the disease, whether the antigen binding molecule as described herein is administered for preventive or therapeutic purposes, previous or concurrent therapeutic interventions, the patient's clinical history and response to the bispecific antigen binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The antigen binding molecule or IFNG variant polypeptide as disclosed herein is suitably administered to the patient at one time or over a series of treatments. Depending on the type and severity of the disease, about 1 μg/kg to 15 mg/kg (e.g. 0.1 mg/kg-10 mg/kg) of the antigen binding molecule or IFNG variant polypeptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antigen binding molecule or IFNG variant polypeptide would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 0.1 mg/kg body weight to about 20 mg/kg body weight, about 5 μg/kg body weight to about 1 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). In a particular aspect, the bispecific antigen binding molecule will be administered every three weeks. An initial higher loading dose, followed by one or more lower doses may be administered. However, other dosage regimens may be useful. The progress of this therapy is easily monitored by conventional techniques and assays.

The antigen binding molecule or IFNG variant polypeptide described herein will generally be used in an amount effective to achieve the intended purpose. For use to treat or prevent a disease condition, the antigen binding molecule or IFNG variant polypeptide, or pharmaceutical compositions thereof, are administered or applied in a therapeutically effective amount. Determination of a therapeutically effective amount is well within the capabilities of those skilled in the art, especially in light of the detailed disclosure provided herein. For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the antigen binding molecule or IFNG variant polypeptide disclosed herein which are sufficient to maintain therapeutic effect. Usual patient dosages for administration by injection range from about 0.1 to 50 mg/kg/day, typically from about 0.1 to 1 mg/kg/day. Therapeutically effective plasma levels may be achieved by administering multiple doses each day. Levels in plasma may be measured, for example, by HPLC. In cases of local administration or selective uptake, the effective local concentration of the antigen binding molecule or IFNG variant polypeptide may not be related to plasma concentration. One skilled in the art will be able to optimize therapeutically effective local dosages without undue experimentation.

A therapeutically effective dose of the antigen binding molecule or IFNG variant polypeptide described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Antigen binding molecules that exhibit large therapeutic indices are preferred. In one aspect, the antigen binding molecule or IFNG variant polypeptide as disclosed herein exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with antigen binding molecules would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The antigen binding molecule or IFNG variant polypeptide as disclosed herein may be administered in combination with one or more other agents in therapy. For instance, the antigen binding molecule or IFNG variant polypeptide may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In certain aspects, an additional therapeutic agent is an immunomodulatory agent, a cytostatic agent, an inhibitor of cell adhesion, a cytotoxic or cytostatic agent, an activator of cell apoptosis, or an agent that increases the sensitivity of cells to apoptotic inducers.

Thus, provided are antigen binding molecules or IFNG variant polypeptides as described herein or pharmaceutical compositions comprising them for use in the treatment of cancer, wherein the antigen binding molecules or IFNG variant polypeptides are administered in combination with a chemotherapeutic agent, radiation and/or other agents for use in cancer immunotherapy.

Such other agents are suitably present in combination in amounts that are effective for the purpose intended. The effective amount of such other agents depends on the amount of bispecific antibody used, the type of disorder or treatment, and other factors discussed above. The bispecific antigen binding molecule or antibody of the invention are generally used in the same dosages and with administration routes as described herein, or about from 1 to 99% of the dosages described herein, or in any dosage and by any route that is empirically/clinically determined to be appropriate. Such combination therapies noted above encompass combined administration (where two or more therapeutic agents are included in the same or separate compositions), and separate administration, in which case, administration of the antigen binding molecule or IFNG variant polypeptide can occur prior to, simultaneously, and/or following, administration of the additional therapeutic agent and/or adjuvant.

In a further aspect, provided is the antigen binding molecule or IFNG variant polypeptide as described herein before for use in the treatment of cancer, wherein the antigen binding molecule or IFNG variant polypeptide is administered in combination with another immunomodulator.

The term “immunomodulator” refers to any substance including a monoclonal antibody that effects the immune system. The molecules described herein can be considered immunomodulators. Immunomodulators can be used as anti-neoplastic agents for the treatment of cancer. In one aspect, immunomodulators include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), LAG3 antibodies (relatlimab), PD1-LAG3 bispecific antibodies or TIGIT antibodies (tiragolumab). In a further aspect, provided is the antigen binding molecule or IFNG variant polypeptide as described herein for use in the treatment of cancer, wherein the antigen binding molecule or IFNG variant polypeptide is administered in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is an antibody selected from the group consisting of atezolizumab, durvalumab, pembrolizumab and nivolumab. In one specific aspect, the agent blocking PD-L1/PD-1 interaction is atezolizumab (MPDL3280A, RG7446). In another aspect, the agent blocking PD-L1/PD-1 interaction is an anti-PD1 antibody, in particular an anti-PD1 antibody selected from pembrolizumab or nivolumab. In another aspect, the agent blocking PD-L1/PD-1 interaction is an anti-PD1/anti-LAG3 bispecific antibody. Such other agents are suitably present in combination in amounts that are effective for the purpose intended. The effective amount of such other agents depends on the amount of antigen binding molecule used, the type of disorder or treatment, and other factors discussed above. The antigen binding molecules or IFNG variant polypeptides as described herein before are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antigen binding molecule can occur prior to, simultaneously, and/or following, administration of the additional therapeutic agent and/or adjuvant.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an antigen binding molecule as described herein.

The label or package insert indicates that the composition is used for treating the condition of choice. Moreover, the article of manufacture may comprise (a) a first container with a composition contained therein, wherein the composition comprises the antigen binding molecule as described herein; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Name | Sequence |
| 1 | IFNG C-terminal | KRKRP |
| | sequence | |
| 2 | Human IFNG- | QDPYVKEAENLKKYFNAGHSDVADNGTLFLGILKNWKEESDRKIMQSQI |
| | KRKRP | VSFYFKLFKNFKDDQSIQKSVETIKEDMNVKFFNSNKKKRDDFEKLTNY |
| | | SVTDLNVQRKAIHELIQVMAELSPAAKTGKRKRP |
| 3 | Mouse IFNG- | HGTVIESLESLNNYFNSSGIDVEEKSLFLDIWRNWQKDGDMKILQSQII |
| | KRKRP | SFYLRLFEVLKDNQAISNNISVIESHLITTFFSNSKAKKDAFMSIAKFE |
| | | VNNPQVQRQAFNELIRVVHQLLPESSLRKRKRP |
| 4 | FAP(4B9) | SYAMS |
| | CDR-H1 | |
| 5 | FAP(4B9) | AIIGSGASTYYADSVKG |
| | CDR-H2 | |
| 6 | FAP(4B9) | GWFGGFNY |
| | CDR-H3 | |
| 7 | FAP(4B9) | RASQSVTSSYLA |
| | CDR-L1 | |
| 8 | FAP(4B9) | VGSRRAT |
| | CDR-L2 | |
| 9 | FAP(4B9) | QQGIMLPPT |
| | CDR-L3 | |
| 10 | FAP(4B9) VH | EVQLLESGGGLVQPGGSLRLSCAASGFTFSSYAMSWVRQAPGKGLEWVS |
| | | AIIGSGASTYYADSVKGRFTISRDNSKNTLYLQMNSLRAEDTAVYYCAK |
| | | GWFGGFNYWGQGTLVTVSS |
| 11 | FAP(4B9) VL | EIVLTQSPGTLSLSPGERATLSCRASQSVTSSYLAWYQQKPGQAPRLLI |
| | | NVGSRRATGIPDRFSGSGSGTDFTLTISRLEPEDFAVYYCQQGIMLPPT |
| | | FGQGTKVEIK |
| 12 | FAP (28H1) | SHAMS |
| | CDR-H1 | |
| 13 | FAP (28H1) | AIWASGEQYYADSVKG |
| | CDR-H2 | |
| 14 | FAP (28H1) | GWLGNEDY |
| | CDR-H3 | |
| 15 | FAP (28H1) | RASQSVSRSYLA |
| | CDR-L1 | |
| 16 | FAP (28H1) | GASTRAT |
| | CDR-L2 | |
| 17 | FAP (28H1) | QQGQVIPPT |
| | CDR-L3 | |
| 18 | FAP(28H1) VH | EVQLLESGGGLVQPGGSLRLSCAASGFTFSSHAMSWVRQAPGKGLEWVS |
| | | AIWASGEQYYADSVKGRFTISRDNSKNTLYLQMNSLRAEDTAVYYCAKG |
| | | WLGNFDYWGQGTLVTVSS |
| 19 | FAP(28H1) VL | EIVLTQSPGTLSLSPGERATLSCRASQSVSRSYLAWYQQKPGQAPRLLI |
| | | IGASTRATGIPDRFSGSGSGTDFTLTISRLEPEDFAVYYCQQGQVIPPT |
| | | FGQGTKVEIK |
| 20 | Protease | PQARK |
| | recognition site 1 | |
| 21 | Protease | HQARK |
| | recognition site 2 | |
| 22 | anti-hu IFNG | SSWIN |
| | CDR-H1 | |
| 23 | anti-hu IFNG | RIDPSDGEVHYNQDFKD |
| | CDR-H2 | |
| 24 | anti-hu IFNG | GFLPWFAD |
| | CDR-H3 | |
| 25 | anti-hu IFNG | KASENVDTYVS |
| | CDR-L1 | |
| 26 | anti-hu IFNG | GASNRYT |
| | CDR-L2 | |
| 27 | anti-hu IFNG | GQSYNYPFT |
| | CDR-L3 | |
| 28 | anti-hu IFNG | EVQLVQSGAELKKPGSSVKVSCKASGYIFTSSWINWVKQAPGQCLEWIG |
| | VH | RIDPSDGEVHYNQDFKDKATLTVDKSTNTAYMELSSLRSEDTAVYYCAR |
| | | GFLPWFADWGQGTLVTVSS |
| 29 | anti-hu IFNG VL | DIQMTQSPSTLSASVGDRVTITCKASENVDTYVSWYQQKPGKAPKLLIY |
| | | GASNRYTGVPSRFSGSGSGTDFTLTISSLQPDDFATYYCGQSYNYPFTE |
| | | GCGTKVEVK |
| 30 | anti-mu IFNG | TNSVH |
| | CDR-H1 | |
| 31 | anti-mu IFNG | AVWTDGTTDYNSALKS |
| | CDR-H2 | |
| 32 | anti-mu IFNG | EHVYYGLV |
| | CDR-H3 | |
| 33 | anti-mu IFNG | KASQNVGNNIA |
| | CDR-L1 | |
| 34 | anti-mu IFNG | YASNRFT |
| | CDR-L2 | |
| 35 | anti-mu IFNG | QRIFNAPNT |
| | CDR-L3 | |
| 36 | anti-mu IFNG | QVQLQESGPGLVQASQPLSLTCTVSGFSLTTNSVHWIRQPAGKCLEWMG |
| | VH | AVWTDGTTDYNSALKSRLTISRDTSKSQVFLEMSSLQTEDVATYYCARE |
| | | HVYYGLVGWGQGTLVTVSA |
| 37 | anti-mu IFNG | NIVMTQSPKSMSISVGDRVTMNCKASQNVGNNIAWYQQNPGQSPKLLIY |
| | VL | YASNRFTGVPDRFTGGGYGTDFTLTINNVQTEDAASYYCQRIFNAPNTE |
| | | GCGTKLELK |
| 38 | Anti-hu IFNG | EVQLVQSGAELKKPGSSVKVSCKASGYIFTSSWINWVKQAPGQCLEWIG |
| | scFv | RIDPSDGEVHYNQDFKDKATLTVDKSTNTAYMELSSLRSEDTAVYYCAR |
| | | GFLPWFADWGQGTLVTVSSGGGGSGGGGSGGGGSDIQMTQSPSTLSASV |
| | | GDRVTITCKASENVDTYVSWYQQKPGKAPKLLIYGASNRYTGVPSRFSG |
| | | SGSGTDFTLTISSLQPDDFATYYCGQSYNYPFTFGCGTKVEVK |
| 39 | Anti-mu IFNG | QVQLQESGPGLVQASQPLSLTCTVSGFSLTTNSVHWIRQPAGKCLEWMG |
| | scFv | AVWTDGTTDYNSALKSRLTISRDTSKSQVELEMSSLQTEDVATYYCARE |
| | | HVYYGLVGWGQGTLVTVSAGGGGSGGGGSGGGGSNIVMTQSPKSMSISV |
| | | GDRVTMNCKASQNVGNNIAWYQQNPGQSPKLLIYYASNRFTGVPDRFTG |
| | | GGYGTDFTLTINNVQTEDAASYYCQRIFNAPNTFGCGTKLELK |
| 40 | P1AG7568 | EVQLLESGGGLVQPGGSLRLSCAASGFTESSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVELFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSGGGGSGGGGSGGGGGQDPYVKEAENLKKY |
| | | FNAGHSDVADNGTLFLGILKNWKEESDRKIMQSQIVSFYFKLFKNFKDD |
| | | QSIQKSVETIKEDMNVKFFNSNKKKRDDFEKLTNYSVTDLNVQRKAIHE |
| | | LIQVMAELSPAAKTGKRKRPGGGGSGGGGSGGGPQARKGGGGGGSGGGG |
| | | GEVQLVQSGAELKKPGSSVKVSCKASGYIFTSSWINWVKQAPGQCLEWI |
| | | GRIDPSDGEVHYNQDFKDKATLTVDKSTNTAYMELSSLRSEDTAVYYCA |
| | | RGFLPWFADWGQGTLVTVSSGGGGSGGGGSGGGGSDIQMTQSPSTLSAS |
| | | VGDRVTITCKASENVDTYVSWYQQKPGKAPKLLIYGASNRYTGVPSRFS |
| | | GSGSGTDFTLTISSLQPDDFATYYCGQSYNYPFTFGCGTKVEVK |
| 41 | FAP (4B9) light | EIVLTQSPGTLSLSPGERATLSCRASQSVTSSYLAWYQQKPGQAPRLLI |
| | chain | NVGSRRATGIPDRFSGSGSGTDFTLTISRLEPEDFAVYYCQQGIMLPPT |
| | | FGQGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACE |
| | | VTHQGLSSPVTKSFNRGEC |
| 42 | P1AG7571 | EVQLLESGGGLVQPGGSLRLSCAASGFTFSSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSGGGGSGGGGSGGGGGEVQLVQSGAELKKP |
| | | GSSVKVSCKASGYIFTSSWINWVKQAPGQCLEWIGRIDPSDGEVHYNQD |
| | | FKDKATLTVDKSTNTAYMELSSLRSEDTAVYYCARGELPWFADWGQGTL |
| | | VTVSSGGGGSGGGGSGGGGSDIQMTQSPSTLSASVGDRVTITCKASENV |
| | | DTYVSWYQQKPGKAPKLLIYGASNRYTGVPSRFSGSGSGTDFTLTISSL |
| | | QPDDFATYYCGQSYNYPFTFGCGTKVEVKGGGGSGGGGSGGGPQARKGG |
| | | GGGGSGGGGGQDPYVKEAENLKKYFNAGHSDVADNGTLFLGILKNWKEE |
| | | SDRKIMQSQIVSFYFKLFKNFKDDQSIQKSVETIKEDMNVKFENSNKKK |
| | | RDDFEKLTNYSVTDLNVQRKAIHELIQVMAELSPAAKTGKRKRP |
| 43 | P1AG3766 | EVQLLESGGGLVQPGGSLRLSCAASGFTFSSHAMSWVRQAPGKGLEWVS |
| | heavy chain | AIWASGEQYYADSVKGRFTISRDNSKNTLYLQMNSLRAEDTAVYYCAKG |
| | | WLGNFDYWGQGTLVTVSSAKTTPPSVYPLAPGSAAQTNSMVTLGCLVKG |
| | | YFPEPVTVTWNSGSLSSGVHTFPAVLQSDLYTLSSSVTVPSSTWPSQTV |
| | | TCNVAHPASSTKVDKKIVPRDCGCKPCICTVPEVSSVFIFPPKPKDVLT |
| | | ITLTPKVTCVVVAISKDDPEVQFSWFVDDVEVHTAQTKPREEQINSTFR |
| | | SVSELPIMHQDWLNGKEFKCRVNSAAFGAPIEKTISKTKGRPKAPQVYT |
| | | IPPPKEQMAKDKVSLTCMITNFFPEDITVEWQWNGQPAENYKNTQPIMD |
| | | TDGSYFVYSKLNVQKSNWEAGNTFTCSVLHEGLHNHHTEKSLSHSPGKG |
| | | GGGSGGGGSGGGGSGGGGSGGGGSGGGGGHGTVIESLESLNNYFNSSGI |
| | | DVEEKSLFLDIWRNWQKDGDMKILQSQIISFYLRLFEVLKDNQAISNNI |
| | | SVIESHLITTFFSNSKAKKDAFMSIAKFEVNNPQVQRQAFNELIRVVHQ |
| | | LLPESSLRKRKRPGGGGSGGGGSGGGPQARKGGGGGGSGGGGGQVQLQE |
| | | SGPGLVQASQPLSLTCTVSGFSLTTNSVHWIRQPAGKCLEWMGAVWTDG |
| | | TTDYNSALKSRLTISRDTSKSQVFLEMSSLQTEDVATYYCAREHVYYGL |
| | | VGWGQGTLVTVSAGGGGSGGGGSGGGGSNIVMTQSPKSMSISVGDRVTM |
| | | NCKASQNVGNNIAWYQQNPGQSPKLLIYYASNRFTGVPDRFTGGGYGTD |
| | | FTLTINNVQTEDAASYYCQRIFNAPNTFGCGTKLELK |
| 44 | FAP (28H1) mu | EIVLTQSPGTLSLSPGERATLSCRASQSVSRSYLAWYQQKPGQAPRLLI |
| | light chain | IGASTRATGIPDRFSGSGSGTDFTLTISRLEPEDFAVYYCQQGQVIPPT |
| | | FGQGTKVEIKRADAAPTVSIFPPSSEQLTSGGASVVCFLNNFYPKDINV |
| | | KWKIDGSERQNGVLNSWTDQDSKDSTYSMSSTLTLTKDEYERHNSYTCE |
| | | ATHKTSTSPIVKSFNRNEC |
| 45 | P1AG3755 | EVQLLESGGGLVQPGGSLRLSCAASGFTFSSHAMSWVRQAPGKGLEWVS |
| | heavy chain | AIWASGEQYYADSVKGRFTISRDNSKNTLYLQMNSLRAEDTAVYYCAKG |
| | | WLGNFDYWGQGTLVTVSSAKTTPPSVYPLAPGSAAQTNSMVTLGCLVKG |
| | | YFPEPVTVTWNSGSLSSGVHTFPAVLQSDLYTLSSSVTVPSSTWPSQTV |
| | | TCNVAHPASSTKVDKKIVPRDCGCKPCICTVPEVSSVFIFPPKPKDVLT |
| | | ITLTPKVTCVVVAISKDDPEVQFSWFVDDVEVHTAQTKPREEQINSTFR |
| | | SVSELPIMHQDWLNGKEFKCRVNSAAFGAPIEKTISKTKGRPKAPQVYT |
| | | IPPPKEQMAKDKVSLTCMITNFFPEDITVEWQWNGQPAENYKNTQPIMD |
| | | TDGSYFVYSKLNVQKSNWEAGNTFTCSVLHEGLHNHHTEKSLSHSPGKG |
| | | GGGSGGGGSGGGGSGGGGSGGGGSGGGGGQVQLQESGPGLVQASQPLSL |
| | | TCTVSGFSLTTNSVHWIRQPAGKCLEWMGAVWTDGTTDYNSALKSRLTI |
| | | SRDTSKSQVELEMSSLQTEDVATYYCAREHVYYGLVGWGQGTLVTVSAG |
| | | GGGSGGGGSGGGGSNIVMTQSPKSMSISVGDRVTMNCKASQNVGNNIAW |
| | | YQQNPGQSPKLLIYYASNRFTGVPDRFTGGGYGTDETLTINNVQTEDAA |
| | | SYYCQRIFNAPNTFGCGTKLELKGGGGSGGGGSGGGPQARKGGGGGGSG |
| | | GGGGHGTVIESLESLNNYFNSSGIDVEEKSLELDIWRNWQKDGDMKILQ |
| | | SQIISFYLRLFEVLKDNQAISNNISVIESHLITTFFSNSKAKKDAFMSI |
| | | AKFEVNNPQVQRQAFNELIRVVHQLLPESSLRKRKRP |
| 46 | Human FAP, | MKTWVKIVFG VATSAVLALL VMCIVLRPSR VHNSEENTMR |
| | UniProt Q12884 | ALTLKDILNG TFSYKTFFPN WISGQEYLHQ SADNNIVLYN |
| | | IETGQSYTIL SNRTMKSVNA SNYGLSPDRQ FVYLESDYSK |
| | | LWRYSYTATY YIYDLSNGEF VRGNELPRPI QYLCWSPVGS |
| | | KLAYVYQNNI YLKQRPGDPP FQITFNGREN KIFNGIPDWV |
| | | YEEEMLATKY ALWWSPNGKF LAYAEFNDTD IPVIAYSYYG |
| | | DEQYPRTINI PYPKAGAKNP VVRIFIIDTT YPAYVGPQEV |
| | | PVPAMIASSD YYFSWLTWVT DERVCLQWLK RVQNVSVLSI |
| | | CDFREDWQTW DCPKTQEHIE ESRTGWAGGF FVSTPVFSYD |
| | | AISYYKIFSD KDGYKHIHYI KDTVENAIQI TSGKWEAINI |
| | | FRVTQDSLFY SSNEFEEYPG RRNIYRISIG SYPPSKKCVT |
| | | CHLRKERCQY YTASFSDYAK YYALVCYGPG IPISTLHDGR |
| | | TDQEIKILEE NKELENALKN IQLPKEEIKK LEVDEITLWY |
| | | KMILPPQFDR SKKYPLLIQV YGGPCSQSVR SVFAVNWISY |
| | | LASKEGMVIA LVDGRGTAFQ GDKLLYAVYR KLGVYEVEDQ |
| | | ITAVRKFIEM GFIDEKRIAI WGWSYGGYVS SLALASGTGL |
| | | FKCGIAVAPV SSWEYYASVY TERFMGLPTK DDNLEHYKNS |
| | | TVMARAEYFR NVDYLLIHGT ADDNVHFQNS AQIAKALVNA |
| | | QVDFQAMWYS DQNHGLSGLS TNHLYTHMTH FLKQCFSLSD |
| 47 | mouse FAP, | MKTWLKTVFG VTTLAALALV VICIVLRPSR VYKPEGNTKR |
| | UniProt P97321 | ALTLKDILNGTFSYKTYFPN WISEQEYLHQ SEDDNIVFYN |
| | | IETRESYIIL SNSTMKSVNA TDYGLSPDRQ FVYLESDYSK |
| | | LWRYSYTATY YIYDLQNGEF VRGYELPRPI QYLCWSPVGS |
| | | KLAYVYQNNI YLKQRPGDPP FQITYTGREN RIFNGIPDWV |
| | | YEEEMLATKY ALWWSPDGKF LAYVEFNDSD IPIIAYSYYG |
| | | DGQYPRTINI PYPKAGAKNP VVRVFIVDTT YPHHVGPMEV |
| | | PVPEMIASSD YYFSWLTWVS SERVCLQWLK RVQNVSVLSI |
| | | CDFREDWHAW ECPKNQEHVE ESRTGWAGGF FVSTPAFSQD |
| | | ATSYYKIFSD KDGYKHIHYI KDTVENAIQI TSGKWEAIYI |
| | | FRVTQDSLFY SSNEFEGYPG RRNIYRISIG NSPPSKKCVT |
| | | CHLRKERCQY YTASFSYKAK YYALVCYGPG LPISTLHDGR |
| | | TDQEIQVLEE NKELENSLRN IQLPKVEIKK LKDGGLTFWY |
| | | KMILPPQFDR SKKYPLLIQV YGGPCSQSVK SVFAVNWITY |
| | | LASKEGIVIA LVDGRGTAFQ GDKFLHAVYR KLGVYEVEDQ |
| | | LTAVRKFIEM GFIDEERIAI WGWSYGGYVS SLALASGTGL |
| | | FKCGIAVAPV SSWEYYASIY SERFMGLPTK DDNLEHYKNS |
| | | TVMARAEYFR NVDYLLIHGT ADDNVHFQNS AQIAKALVNA |
| | | QVDFQAMWYS DQNHGISSGR SQNHLYTHMT HFLKQCFSLS D |
| 48 | CH1 domain | ASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKV |
| 49 | CH1 connector | EPKSC |
| | C-terminal end | |
| 50 | Hinge full | DKTHTCPXCP with X being S or P |
| 51 | Hinge middle | HTCPXCP with X being S or P |
| 52 | Hinge short | CPXCP with X being S or P |
| 53 | CH2 domain | APELLGGPSVFLFPPKPKDTLMISRTPEVTCVWDVSHEDPEVKFNWYVD |
| | | GVEVHNAKTKPREEQESTYRWSVLTVLHQDWLNGKEYKCKVSNKALPAP |
| | | IEKTISKAK |
| 54 | CH3 domain | GQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQ |
| | | KSLSLSP |
| 55 | Fc IgG1, | ASTKGPSVFPLAPSSKSTSGGTAALGCLVKDYFPEPVTVSWNSGALTSG |
| | caucasian | VHTFPAVLQSSGLYSLSSVVTVPSSSLGTQTYICNVNHKPSNTKVDKKV |
| | allotype | EPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLT |
| | | VDKSRWQQGNVFSCSVMHEALHNHYTQKSLSLSPGK |
| 56 | Fc IgG1, | ASTKGPSVFPLAPSSKSTSGGTAALGCLVKDYFPEPVTVSWNSGALTSG |
| | afroamerican | VHTFPAVLQSSGLYSLSSVVTVPSSSLGTQTYICNVNHKPSNTKVDKKV |
| | allotype | EPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLT |
| | | VDKSRWQQGNVFSCSVMHEALHNHYTQKSLSLSPGK |
| 57 | Fc IgG2 | ASTKGPSVFPLAPCSRSTSESTAALGCLVKDYFPEPVTVSWNSGALTSG |
| | | VHTFPAVLQSSGLYSLSSVVTVPSSNFGTQTYTCNVDHKPSNTKVDKTV |
| | | ERKCCVECPPCPAPPVAGPSVFLFPPKPKDTLMISRTPEVTCVVVDVSH |
| | | EDPEVQFNWYVDGVEVHNAKTKPREEQENSTFRVVSVLTVVHQDWLNGK |
| | | EYKCKVSNKGLPAPIEKTISKTKGQPREPQVYTLPPSREEMTKNQVSLT |
| | | CLVKGFYPSDISVEWESNGQPENNYKTTPPMLDSDGSFFLYSKLTVDKS |
| | | RWQQGNVFSCSVMHEALHNHYTQKSLSLSPGK |
| 58 | Fc IgG3 | ASTKGPSVFPLAPCSRSTSGGTAALGCLVKDYFPEPVTVSWNSGALTSG |
| | | VHTFPAVLQSSGLYSLSSVVTVPSSSLGTQTYTCNVNHKPSNTKVDKRV |
| | | ELKTPLGDTTHTCPRCPEPKSCDTPPPCPRCPEPKSCDTPPPCPRCPEP |
| | | KSCDTPPPCPRCPAPELLGGPSVFLFPPKPKDTLMISRTPEVTCVVVDV |
| | | SHEDPEVQFKWYVDGVEVHNAKTKPREEQYNSTFRVVSVLTVLHQDWLN |
| | | GKEYKCKVSNKALPAPIEKTISKTKGQPREPQVYTLPPSREEMTKNQVS |
| | | LTCLVKGFYPSDIAVEWESSGQPENNYNTTPPMLDSDGSFFLYSKLTVD |
| | | KSRWQQGNIFSCSVMHEALHNRFTQKSLSLSPGK |
| 59 | Fc IgG4 | ASTKGPSVFPLAPCSRSTSESTAALGCLVKDYFPEPVTVSWNSGALTSG |
| | | VHTFPAVLQSSGLYSLSSVVTVPSSSLGTKTYTCNVDHKPSNTKVDKRV |
| | | ESKYGPPCPSCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLTVDK |
| | | SRWQEGNVFSCSVMHEALHNHYTQKSLSLSLGK |
| 60 | Human IFNG | MKYTSYILAFQLCIVLGSLGCYCQDPYVKEAENLKKYFNAGHSDVADNG |
| | | TLFLGILKNWKEESDRKIMQSQIVSFYFKLFKNEKDDQSIQKSVETIKE |
| | | DMNVKFFNSNKKKRDDFEKLTNYSVTDLNVQRKAIHELIQVMAELSPAA |
| | | KTGKRKRSQMLERGRRASQ |
| 61 | Mouse IFNG, | MNATHCILALQLELMAVSGCYCHGTVIESLESLNNYENSSGIDVEEKSL |
| | Uniprot P01580 | FLDIWRNWQKDGDMKILQSQIISFYLRLFEVLKDNQAISNNISVIESHL |
| | | ITTFFSNSKAKKDAFMSIAKFEVNNPQVQRQAFNELIRVVHQLLPESSL |
| | | RKRKRSRC |
| 62 | Peptide linker | GGGGS |
| | (G4S) | |
| 63 | Peptide linker | GGGGSGGGGS |
| | (G4S)₂ | |
| 64 | Peptide linker | SGGGGSGGGG |
| | (SG4)₂ | |
| 65 | Peptide linker | GGGGSGGGGSGGGG |
| | G4(SG4)₂ | |
| 66 | (G4S)₃ peptide | GGGGSGGGGSGGGGS |
| | linker | |
| 67 | (G4S)₄ peptide | GGGGSGGGGSGGGGSGGGGS |
| | linker | |
| 68 | (G4S)₅ peptide | GGGGSGGGGSGGGGSGGGGSGGGGS |
| | linker | |
| 69 | (G4S)5GGGGG | GGGGSGGGGSGGGGSGGGGSGGGGSGGGGG |
| | peptide linker | |
| 70 | PQARK linker | GGGGSGGGGSGGGPQARKGGGGGGSGGGGG |
| 71 | HQARK linker | GGGGSGGGGSGGGHQARKGGGGGGSGGGGG |
| 72 | peptide linker | GGGGSGGG |
| 73 | peptide linker A | LEVLFQG |
| 74 | peptide linker B | GGGGSGGGGSGGGGSGGGGSGLEVLFQGPGGGGGSGGGGG |
| 75 | peptide linker C | QARK |
| 76 | Hinge 1 | TCPPCP |
| 77 | Hinge 2 | PCP |
| 78 | KRKR patch | KRKR |
| 79 | P1AG3768 | EVQLLESGGGLVQPGGSLRLSCAASGFTFSSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKRSQMLERG |
| 80 | P1AG3769 | EVQLLESGGGLVQPGGSLRLSCAASGFTFSSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| 81 | P1AF3570 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKRSQ |
| 82 | P1AF3571 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKRSL |
| 83 | P1AF3574 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKRP |
| 84 | P1AF3575 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKP |
| 85 | P1AF3576 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRP |
| 86 | P1AF3577 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KP |
| 87 | P1AF3578 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKRSP |
| 88 | P1AF9470 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKSP |
| 89 | P1AF3580 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNEKDDQSIQKSVETIKEDMN |
| | | VKFFNSNKKKRDDFEKLTNYSVTDLNVQRKAIHELIQVMAELSPAAKTG |
| | | KRSP |
| 90 | P1AF3581 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KSP |
| 91 | P1AF9464 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | SRKRP |
| 92 | P1AF9469 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KSKRP |
| 93 | P1AF9465 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRSRP |
| 94 | P1AF9470 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKSP |
| 95 | P1AF9475 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | PRKRP |
| 96 | P1AF9476 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KPKRP |
| 97 | P1AF9477 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRPRP |
| 98 | P1AF9478 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKPP |
| 99 | P1AF9483 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | ERKRP |
| 100 | P1AF9484 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KEKRP |
| 101 | P1AF9485 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRERP |
| 102 | P1AF9486 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRKEP |
| 103 | P1AF9473 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNEKDDQSIQKSVETIKEDMN |
| | | VKFFNSNKKKRDDFEKLTNYSVTDLNVQRKAIHELIQVMAELSPAAKTG |
| | | SRSRP |
| 104 | P1AF9481 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLEKNEKDDQSIQKSVETIKEDMN |
| | | VKFFNSNKKKRDDFEKLTNYSVTDLNVQRKAIHELIQVMAELSPAAKTG |
| | | PRPRP |
| 105 | P1AF9489 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | ERERP |
| 106 | P1AF9474 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KSKSP |
| 107 | P1AF9482 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KPKPP |
| 108 | P1AF9490 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KEKEP |
| 109 | P1AF9471 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNEKDDQSIQKSVETIKEDMN |
| | | VKFFNSNKKKRDDFEKLTNYSVTDLNVQRKAIHELIQVMAELSPAAKTG |
| | | SSKRP |
| 110 | P1AF9479 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | PPKRP |
| 111 | P1AF9487 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | EEKRP |
| 112 | P1AF9472 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNEKDDQSIQKSVETIKEDMN |
| | | VKFFNSNKKKRDDFEKLTNYSVTDLNVQRKAIHELIQVMAELSPAAKTG |
| | | KRSSP |
| 113 | P1AF9480 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KRPPP |
| 114 | P1AF9488 heavy | EVQLLESGGGLVQPGGSLRLSCAASGFTFSSYAMSWVRQAPGKGLEWVS |
| | chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSQDPYVKEAENLKKYFNAGHSDVADNGTLF |
| | | LGILKNWKEESDRKIMQSQIVSFYFKLFKNFKDDQSIQKSVETIKEDMN |
| | | VKFFNSNKKKRDDFEKLTNYSVTDLNVQRKAIHELIQVMAELSPAAKTG |
| | | KREEP |
| 115 | P1AG7567 | EVQLLESGGGLVQPGGSLRLSCAASGFTFSSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSGGGGSGGGGSGGGGGQDPYVKEAENLKKY |
| | | FNAGHSDVADNGTLFLGILKNWKEESDRKIMQSQIVSFYFKLEKNEKDD |
| | | QSIQKSVETIKEDMNVKFFNSNKKKRDDFEKLTNYSVTDLNVQRKAIHE |
| | | LIQVMAELSPAAKTGKRKRPGGGGSGGGGSGGGGSGGGGSGGGGSGGGG |
| | | GEVQLVQSGAELKKPGSSVKVSCKASGYIFTSSWINWVKQAPGQCLEWI |
| | | GRIDPSDGEVHYNQDFKDKATLTVDKSTNTAYMELSSLRSEDTAVYYCA |
| | | RGFLPWFADWGQGTLVTVSSGGGGSGGGGSGGGGSDIQMTQSPSTLSAS |
| | | VGDRVTITCKASENVDTYVSWYQQKPGKAPKLLIYGASNRYTGVPSRFS |
| | | GSGSGTDFTLTISSLQPDDFATYYCGQSYNYPFTFGCGTKVEVK |
| 116 | P1AG7570 | EVQLLESGGGLVQPGGSLRLSCAASGFTFSSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSGGGGSGGGGSGGGGGEVQLVQSGAELKKP |
| | | GSSVKVSCKASGYIFTSSWINWVKQAPGQCLEWIGRIDPSDGEVHYNQD |
| | | FKDKATLTVDKSTNTAYMELSSLRSEDTAVYYCARGELPWFADWGQGTL |
| | | VTVSSGGGGSGGGGSGGGGSDIQMTQSPSTLSASVGDRVTITCKASENV |
| | | DTYVSWYQQKPGKAPKLLIYGASNRYTGVPSRFSGSGSGTDETLTISSL |
| | | QPDDFATYYCGQSYNYPFTFGCGTKVEVKGGGGSGGGGSGGGGSGGGGS |
| | | GGGGSGGGGGQDPYVKEAENLKKYFNAGHSDVADNGTLFLGILKNWKEE |
| | | SDRKIMQSQIVSFYFKLFKNFKDDQSIQKSVETIKEDMNVKFFNSNKKK |
| | | RDDFEKLTNYSVTDLNVQRKAIHELIQVMAELSPAAKTGKRKRP |
| 117 | P1AG1310 | EVQLLESGGGLVQPGGSLRLSCAASGFTFSSYAMSWVRQAPGKGLEWVS |
| | heavy chain | AIIGSGASTYYADSVKGRFTISRDNSKNTLYLQMNSLRAEDTAVYYCAK |
| | | GWFGGFNYWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALGAPIEKTISKAKGQP |
| | | REPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGGGGGSGGGGSGGGGSGGGGSGGGGSGGGGGQDPYVKEAENLKKY |
| | | FNAGHSDVADNGTLFLGILKNWKEESDRKIMQSQIVSFYFKLEKNEKDD |
| | | QSIQKSVETIKEDMNVKFFNSNKKKRDDFEKLTNYSVTDLNVQRKAIHE |
| | | LIQVMAELSPAAKTGKRKRP |
| 118 | P1AG9672 | EVQLLESGGGLVQPGGSLRLSCAASGFTFSSHAMSWVRQAPGKGLEWVS |
| | heavy chain | AIWASGEQYYADSVKGRFTISRDNSKNTLYLQMNSLRAEDTAVYYCAKG |
| | | WLGNFDYWGQGTLVTVSSAKTTPPSVYPLAPGSAAQTNSMVTLGCLVKG |
| | | YFPEPVTVTWNSGSLSSGVHTFPAVLQSDLYTLSSSVTVPSSTWPSQTV |
| | | TCNVAHPASSTKVDKKIVPRDCGCKPCICTVPEVSSVFIFPPKPKDVLT |
| | | ITLTPKVTCVVVAISKDDPEVQFSWFVDDVEVHTAQTKPREEQINSTER |
| | | SVSELPIMHQDWLNGKEFKCRVNSAAFGAPIEKTISKTKGRPKAPQVYT |
| | | IPPPKEQMAKDKVSLTCMITNFFPEDITVEWQWNGQPAENYKNTQPIMD |
| | | TDGSYFVYSKLNVQKSNWEAGNTFTCSVLHEGLHNHHTEKSLSHSPGKG |
| | | GGGSGGGGSGGGGGHGTVIESLESLNNYFNSSGIDVEEKSLFLDIWRNW |
| | | QKDGDMKILQSQIISFYLRLFEVLKDNQAISNNISVIESHLITTFFSNS |
| | | KAKKDAFMSIAKFEVNNPQVQRQAFNELIRVVHQLLPESSLRKRKRP |
| 119 | P1AG8692 | QVQLVQSGAEVKKPGSSVKVSCKASGFNIKDTYMHWVRQAPGQGLEWMG |
| | heavy chain | RIDPANGNSKYVPKFQGRVTITADTSTSTAYMELSSLRSEDTAVYYCAP |
| | | FGYYVSDYAMAYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCGGGGSGGGGSGGGGSGGGGSG |
| | | GGGSGGGGGQDPYVKEAENLKKYFNAGHSDVADNGTLFLGILKNWKEES |
| | | DRKIMQSQIVSFYFKLFKNFKDDQSIQKSVETIKEDMNVKFENSNKKKR |
| | | DDFEKLTNYSVTDLNVQRKAIHELIQVMAELSPAAKTGKRKRPGGGGSG |
| | | GGTCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGKEYK |
| | | CKVSNKALGAPIEKTISKAKGQPREPQVYTLPPSRDELTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 120 | P1AG8693 | QVQLVQSGAEVKKPGSSVKVSCKASGFNIKDTYMHWVRQAPGQGLEWMG |
| | heavy chain | RIDPANGNSKYVPKFQGRVTITADTSTSTAYMELSSLRSEDTAVYYCAP |
| | | FGYYVSDYAMAYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCGGGGSGGGGSGGGGSGGGGSG |
| | | GGGSGGGGGQDPYVKEAENLKKYFNAGHSDVADNGTLFLGILKNWKEES |
| | | DRKIMQSQIVSFYFKLFKNFKDDQSIQKSVETIKEDMNVKFENSNKKKR |
| | | DDFEKLTNYSVTDLNVQRKAIHELIQVMAELSPAAKTGKRKRPLEVLFQ |
| | | GPTCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGKEYK |
| | | CKVSNKALGAPIEKTISKAKGQPREPQVYTLPPSRDELTKNQVSLTCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 121 | P1AG8694 | QVQLVQSGAEVKKPGSSVKVSCKASGFNIKDTYMHWVRQAPGQGLEWMG |
| | heavy chain | RIDPANGNSKYVPKFQGRVTITADTSTSTAYMELSSLRSEDTAVYYCAP |
| | | FGYYVSDYAMAYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCGGGGSGGGGSGGGGSGGGGSG |
| | | GGGSGGGGGQDPYVKEAENLKKYFNAGHSDVADNGTLFLGILKNWKEES |
| | | DRKIMQSQIVSFYFKLFKNFKDDQSIQKSVETIKEDMNVKFENSNKKKR |
| | | DDFEKLTNYSVTDLNVQRKAIHELIQVMAELSPAAKTGKRKRPLEVLFQ |
| | | GPPCPAPEAAGGPSVFLFPPKPKDTLMISRTPEVTCVVVDVSHEDPEVK |
| | | FNWYVDGVEVHNAKTKPREEQYNSTYRVVSVLTVLHQDWLNGKEYKCKV |
| | | SNKALGAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGN |
| | | VFSCSVMHEALHNHYTQKSLSLSPGK |
| 122 | P1AG8695 | QVQLVQSGAEVKKPGSSVKVSCKASGFNIKDTYMHWVRQAPGQGLEWMG |
| | heavy chain | RIDPANGNSKYVPKFQGRVTITADTSTSTAYMELSSLRSEDTAVYYCAP |
| | | FGYYVSDYAMAYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCGGGGSGGGGSGGGGSGGGGSG |
| | | GGGSGGGGGQDPYVKEAENLKKYFNAGHSDVADNGTLFLGILKNWKEES |
| | | DRKIMQSQIVSFYFKLFKNFKDDQSIQKSVETIKEDMNVKFENSNKKKR |
| | | DDFEKLTNYSVTDLNVQRKAIHELIQVMAELSPAAKTGKRKRPLEVLFQ |
| | | GPAPEAAGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNK |
| | | ALGAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNVES |
| | | CSVMHEALHNHYTQKSLSLSPGK |
| 123 | P1AG8696 | QVQLVQSGAEVKKPGSSVKVSCKASGFNIKDTYMHWVRQAPGQGLEWMG |
| | heavy chain | RIDPANGNSKYVPKFQGRVTITADTSTSTAYMELSSLRSEDTAVYYCAP |
| | | FGYYVSDYAMAYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCGGGGSGGGGSGGGGSGGGGSG |
| | | GGGSGGGGGQDPYVKEAENLKKYFNAGHSDVADNGTLFLGILKNWKEES |
| | | DRKIMQSQIVSFYFKLFKNFKDDQSIQKSVETIKEDMNVKFENSNKKKR |
| | | DDFEKLTNYSVTDLNVQRKAIHELIQVMAELSPAAKTGKRKRPQARKTC |
| | | PPCPAPEAAGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVS |
| | | NKALGAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGNV |
| | | FSCSVMHEALHNHYTQKSLSLSPGK |
| 124 | P1AG8697 | QVQLVQSGAEVKKPGSSVKVSCKASGENIKDTYMHWVRQAPGQGLEWMG |
| | heavy chain | RIDPANGNSKYVPKFQGRVTITADTSTSTAYMELSSLRSEDTAVYYCAP |
| | | FGYYVSDYAMAYWGQGTLVTVSSASTKGPSVFPLAPSSKSTSGGTAALG |
| | | CLVKDYFPEPVTVSWNSGALTSGVHTFPAVLQSSGLYSLSSVVTVPSSS |
| | | LGTQTYICNVNHKPSNTKVDKKVEPKSCGGGGSGGGGSGGGGSGGGGSG |
| | | LEVLFQGPGGGGGSGGGGGQDPYVKEAENLKKYFNAGHSDVADNGTLEL |
| | | GILKNWKEESDRKIMQSQIVSFYFKLFKNFKDDQSIQKSVETIKEDMNV |
| | | KFFNSNKKKRDDFEKLTNYSVTDLNVQRKAIHELIQVMAELSPAAKTGK |
| | | RKRP |
| 125 | P1AF3568 IFNG | AKTGKRKRSQMLERG |
| | C-terminus | |
| 126 | P1AF3569 IFNG | AKTG |
| | C-terminus | |
| 127 | P1AF3570 IFNG | AKTGKRKRSQ |
| | C-terminus | |
| 128 | P1AF3571 IFNG | AKTGKRKRSL |
| | C-terminus | |
| 129 | P1AF3574 IFNG | AKTGKRKRP |
| | C-terminus | |
| 130 | P1AF3575 IFNG | AKTGKRKP |
| | C-terminus | |
| 131 | P1AF3576 IFNG | AKTGKRP |
| | C-terminus | |
| 132 | P1AF3577 IFNG | AKTGKP |
| | C-terminus | |
| 133 | P1AF3578 IFNG | AKTGKRKRSP |
| | C-terminus | |
| 134 | P1AF9470 IFNG | AKTGKRKSP |
| | C-terminus | |
| 135 | P1AF3580 IFNG | AKTGKRSP |
| | C-terminus | |
| 136 | P1AF3581 IFNG | AKTGKSP |
| | C-terminus | |
| 137 | P1AF9464 IFNG | AKTGSRKRP |
| | C-terminus | |
| 138 | P1AF9469 IFNG | AKTGKSKRP |
| | C-terminus | |
| 139 | P1AF9465 IFNG | AKTGKRSRP |
| | C-terminus | |
| 140 | P1AF9475 IFNG | AKTGPRKRP |
| | C-terminus | |
| 141 | P1AF9476 IFNG | AKTGKPKRP |
| | C-terminus | |
| 142 | P1AF9477 IFNG | AKTGKRPRP |
| | C-terminus | |
| 143 | P1AF9478 IFNG | AKTGKRPPP |
| | C-terminus | |
| 144 | P1AF9483 IFNG | AKTGERKRP |
| | C-terminus | |
| 145 | P1AF9484 IFNG | AKTGKEKRP |
| | C-terminus | |
| 146 | P1AF9485 IFNG | AKTGKRERP |
| | C-terminus | |
| 147 | P1AF9486 IFNG | AKTGKRKEP |
| | C-terminus | |
| 148 | P1AF9473 IFNG | AKTGSRSRP |
| | C-terminus | |
| 149 | P1AF9481 IFNG | AKTGPRPRP |
| | C-terminus | |
| 150 | P1AF9489 IFNG | AKTGERERP |
| | C-terminus | |
| 151 | P1AF9474 IFNG | AKTGKSKSP |
| | C-terminus | |
| 152 | P1AF9482 IFNG | AKTGKPKPP |
| | C-terminus | |
| 153 | P1AF9490 IFNG | AKTGKEKEP |
| | C-terminus | |
| 154 | P1AF9471 IFNG | AKTGSSKRP |
| | C-terminus | |
| 155 | P1AF9479 IFNG | AKTGPPKRP |
| | C-terminus | |
| 156 | P1AF9487 IFNG | AKTGEEKRP |
| | C-terminus | |
| 157 | P1AF9472 IFNG | AKTGKRSSP |
| | C-terminus | |
| 158 | P1AF9480 IFNG | AKTGKRPPP |
| | C-terminus | |
| 159 | P1AF9488 IFNG | AKTGKREEP |
| | C-terminus | |
| 160 | IFNG C- | AKTGKRKRSQMLER |
| | terminus | |
| | shortened by 1 | |
| | amino acid | |
| 161 | IFNG C- | AKTGKRKRSQMLF |
| | terminus | |
| | shortened by 2 | |
| | amino acids | |
| 162 | IFNG C- | AKTGKRKRSQML |
| | terminus | |
| | shortened by 3 | |
| | amino acids | |
| 163 | IFNG C- | AKTGKRKRSQM |
| | terminus | |
| | shortened by 4 | |
| | amino acids | |
| 164 | IFNG C- | AKTGKRK |
| | terminus | |
| | shortened by 8 | |
| | amino acids | |
| 165 | IFNG C- | AKTGKR |
| | terminus | |
| | shortened by 9 | |
| | amino acids | |
| 166 | IFNG C- | AKTGK |
| | terminus | |
| | shortened by 10 | |
| | amino acids | |

The following paragraphs

(paras) are aspects of the invention:

1. An antigen binding molecule, comprising
(i) an antibody that specifically binds to a tumor associated antigen and
(ii) a homodimer of an interferon gamma (IFNG) variant polypeptide, wherein the IFNG variant polypeptide is characterized by the C-terminal amino acid sequence KRKRP (SEQ ID NO: 1).

2. The antigen binding molecule of para 1, wherein the IFNG variant polypeptide comprises or consists of the amino acid sequence of SEQ ID NO:2 or SEQ ID NO:3.

3. The antigen binding molecule of paras 1 or 2, wherein the antibody that specifically binds to a tumor associated antigen is an antibody that specifically binds to Fibroblast activation protein (FAP).

4. The antigen binding molecule of any one of paras 1 to 3, wherein the antibody that specifically binds to FAP comprises
(a) a heavy chain variable region (V_{H}FAP) comprising a heavy chain complementary determining region (CDR-H1) comprising the amino acid sequence of SEQ ID NO:4, a CDR-H2 comprising the amino acid sequence of SEQ ID NO:5, and a CDR-H3 comprising the amino acid sequence of SEQ ID NO:6, and a light chain variable region (V_{L}FAP) comprising a light chain complementarity determining region (iv) CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9, or
(b) a heavy chain variable region (V_{H}FAP) comprising a CDR-H1 comprising the amino acid sequence of SEQ ID NO: 12, a CDR-H2 comprising the amino acid sequence of SEQ ID NO: 13, and a CDR-H3 comprising the amino acid sequence of SEQ ID NO:14, and a light chain variable region (V_{L}FAP) comprising a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO: 17.

5. The antigen binding molecule of any one of paras 1 to 4, wherein the antibody that specifically binds to FAP comprises a heavy chain variable region (V_{H}FAP) comprising the amino acid sequence of SEQ ID NO:10 and a light chain variable region (V_{L}FAP) comprising the amino acid sequence of SEQ ID NO: 11 or it comprises a heavy chain variable region (V_{H}FAP) comprising the amino acid sequence of SEQ ID NO:18 and a light chain variable region (V_{L}FAP) comprising the amino acid sequence of SEQ ID NO: 19.

6. The antigen binding molecule of any one of paras 1 to 5, wherein the antigen binding molecule comprises an IgG1 Fc domain or an IgG4 Fc domain and wherein the Fc domain comprises one or more amino acid substitution that reduces the binding affinity of the antibody to an Fc receptor and/or effector function.

7. The antigen binding molecule of any one of paras 1 to 6, wherein the Fc domain is of human IgG1 subclass with the amino acid mutations L234A, L235A and P329G (EU numbering according to Kabat EU index).

8. The antigen binding molecule of any one of paras 1 to 5, wherein the antigen binding molecule is protease-activatable and comprises a protease recognition site and a masking moiety.

9. The antigen binding molecule of para 8, wherein the protease recognition site is a substrate for matriptase.

10. The antigen binding molecule of paras 8 or 9, wherein the protease recognition site comprises or consists of PQARK (SEQ ID NO:20) or HQARK (SEQ ID NO:21), in particular PQARK (SEQ ID NO:20).

11. The antigen binding molecule of any one of paras 8 to 10, wherein the protease recognition site is part of a cleavable peptide linker which connects the masking moiety with the IFNG variant polypeptide.

12. The antigen binding molecule of any one of paras 8 to 11, wherein the masking moiety is fused at its N-terminus to the C-terminus of the IFNG variant polypeptide via the cleavable peptide linker.

13. The antigen binding molecule of any one of paras 8 to 11, wherein the masking moiety is fused at its N-terminus to the C-terminus of the Fc domain via a stable linker and at its C-terminus to the N-terminus of the IFNG variant polypeptide via the cleavable peptide linker.

14. The antigen binding molecule of any one of paras 8 to 13, wherein the masking moiety is an antibody or antibody fragment that specifically binds to IFNG.

15. The antigen binding molecule of any one of paras 8 to 14, wherein the masking moiety is an scFv that specifically binds to IFNG.

16. The antigen binding molecule of any one of paras 8 to 15, wherein the scFv that specifically binds to IFNG comprises
(a) a heavy chain variable region (V_{H}IFNG) comprising a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24, and a light chain variable region (V_{L}IFNG) comprising a light chain complementarity determining region (iv) CDR-L1 comprising the amino acid sequence of SEQ ID NO:25, a CDR-L2 comprising the amino acid sequence of SEQ ID NO:26, and a CDR-L3 comprising the amino acid sequence of SEQ ID NO:27, or
(b) a heavy chain variable region (V_{H}IFNG) comprising a CDR-H1 comprising the amino acid sequence of SEQ ID NO:30, a CDR-H2 comprising the amino acid sequence of SEQ ID NO:31, and a CDR-H3 comprising the amino acid sequence of SEQ ID NO:32, and a light chain variable region (V_{L}IFNG) comprising a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35.

17. The bispecific antigen binding molecule of any one of paras 1 to 16, wherein scFv that specifically binds to IFNG comprises
(a) a heavy chain variable region (V_{H} IFNG) comprising the amino acid sequence of SEQ ID NO:28 and a light chain variable region (V_{L} IFNG) comprising the amino acid sequence of SEQ ID NO:29, or
(b) a heavy chain variable region (V_{H} IFNG) comprising the amino acid sequence of SEQ ID NO:36 and a light chain variable region (V_{L} IFNG) comprising the amino acid sequence of SEQ ID NO:37.

18. An antigen binding molecule containing an IFNG variant polypeptide, wherein said antigen binding molecule comprises
(i) two heavy chains comprising the amino acid sequence of SEQ ID NO:40 and two light chains comprising the amino acid sequence of SEQ ID NO:41, or (ii) two heavy chains comprising the amino acid sequence of SEQ ID NO:42 and two light chains comprising the amino acid sequence of SEQ ID NO:41, or
(iii) two heavy chains comprising the amino acid sequence of SEQ ID NO:43 and two light chains comprising the amino acid sequence of SEQ ID NO:44, or
(iv) two heavy chains comprising the amino acid sequence of SEQ ID NO:45 and two light chains comprising the amino acid sequence of SEQ ID NO:44.

19. An interferon gamma (IFNG) variant polypeptide, wherein the IFNG variant polypeptide is characterized by the C-terminal amino acid sequence KRKRP (SEQ ID NO:1).

20. The IFNG variant polypeptide of para 19, wherein the IFNG variant polypeptide comprises or consists of the amino acid sequence of SEQ ID NO:2 or SEQ ID NO:3.

21. One or more isolated polynucleotide encoding the antigen binding molecule of any one of paras 1 to 18 or the IFNG variant polypeptide of paras 19 or 20.

22. An expression vector comprising the one or more isolated polynucleotide of para 21.

23. A prokaryotic or eukaryotic host cell comprising the one or more isolated polynucleotide of para 21 or the expression vector of para 22.

24. A method of producing an antigen binding molecule or IFNG variant polypeptide, comprising the steps of a) culturing the prokaryotic or eukaryotic host cell of para 23 under conditions suitable for the expression of the antigen binding molecule or IFNG variant polypeptide and b) optionally recovering the antigen binding molecule or IFNG variant polypeptide.

25. A pharmaceutical composition comprising the antigen binding molecule of any one of paras 1 to 18 or the IFNG variant polypeptide of paras 19 or 20 and a pharmaceutically acceptable excipient.

26. The antigen binding molecule of any one of paras 1 to 17 or the IFNG variant polypeptide of paras 19 or 20 for use as a medicament.

27. The antigen binding molecule of any one of paras 1 to 17 or the IFNG variant polypeptide of paras 19 or 20 for use in the treatment of cancer.

28. The antigen binding molecule of any one of paras 1 to 17 or the IFNG variant polypeptide of paras 19 or 20 for use in the treatment of cancer, wherein the antigen binding molecule or the IFNG variant polypeptide is for administration in combination with a chemotherapeutic agent, radiation and/or other agents for use in cancer immunotherapy.

29. Use of the antigen binding molecule of any one of paras 1 to 17 or the IFNG variant polypeptide of paras 19 or 20 for manufacture of a medicament for treating cancer.

30. A method of treating a disease in an individual, comprising administering to said individual a therapeutically effective amount of the antigen binding molecule of any one of paras 1 to 17 or the IFNG variant polypeptide of paras 19 or 20 in a pharmaceutically acceptable form.

31. The method of para 30, wherein said disease is cancer.

### EXAMPLES

The following are examples of methods and compositions of the invention. It is understood that various other embodiments may be practiced, given the general description provided herein.

### Recombinant DNA Techniques and Sequencing

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242. DNA sequences were determined by double strand sequencing.

### Gene Synthesis

Desired gene segments were either generated by PCR using appropriate templates or were synthesised by GenScript (China) from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration was determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Protein Purification

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralisation of the sample. The protein was concentrated by centrifugation (Millipore Amicon® ULTRA-15 (Art.Nr.: UFC903096), and aggregated protein was separated from monomeric protein by size exclusion chromatography (Superdex 200, GE Healthcare) in 20 mM histidine, 140 mM sodium chloride, pH 6.0. Monomeric compound fractions were pooled, concentrated (if required) using e.g., a MILLIPORE Amicon Ultra (30 MWCO) centrifugal concentrator, frozen and stored at −80° C. Part of the samples were provided for subsequent protein analytics and analytical characterization by CE-SDS, size exclusion chromatography (SE-HPLC) and mass spectrometry (LC-MS).

Alternatively, gene synthesis, cloning, transfection and harvest were outsourced to evitria AG (Schlieren, Switzerland). The corresponding cDNAs were cloned into evitria's vector system using conventional (non-PCR based) cloning techniques. The evitria vector plasmids were gene synthesised. Plasmid DNA was prepared under low-endotoxin conditions based on anion exchange chromatography. DNA concentration was determined by measuring the absorption at a wavelength of 260 nm. Correctness of the sequences was verified with Sanger sequencing (with two sequencing reactions per plasmid). Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 μm filter).

Alternatively, the compounds of interest were prepared by WuXi Biologics using their proprietary vector system with conventional (non-PCR based) cloning techniques and using suspension-adapted HEK293 cells. Expression of all genes was under control of a human CMV promoter. For the production, WuXi Biologics used commercially available chemically defined media and cultivated the cells after transfection at 36.5° C. and 6% carbon dioxide. The supernatant was harvested by centrifugation and subsequent filtration (0.2 μm filter) and proteins were purified from the harvested supernatant by standard methods.

Quantification of Fc containing constructs in supernatants was performed by Protein A—HPLC on an Agilent HPLC System with UV detector. Supernatants were injected on POROS 20 A (Applied Biosystems). The eluted peak area at 280 nm was integrated and converted to concentration by use of a calibration curve with standards analysed in the same run.

Proteins were purified from filtered cell culture supernatants referring to standard protocols. In brief, Fc containing proteins were purified from cell culture supernatants by Protein A-affinity chromatography. Elution was followed by immediate pH neutralisation of the sample. The protein was concentrated by centrifugation (Millipore Amicon® ULTRA-15 (Art.Nr.: UFC903096), and aggregated protein was separated from monomeric protein by size exclusion chromatography (Äkta Pure & HiLoad 26/600 Superdex 200; both from Cytiva) in 20 mM histidine, 140 mM sodium chloride, pH 6.0.

The concentrations of purified proteins were determined by measuring the absorption at 280 nm (Little Lunatic, Unchained labs) using the mass extinction coefficient calculated on the basis of the amino acid sequence according to Pace et al., Protein Sci. 1995, 4 (11), 2411-2423. Purity and molecular weight of the proteins were analysed by CE-SDS in the presence and absence of a reducing agent using a LabChipGXII (Perkin Elmer). Determination of the aggregate content was performed by HPLC chromatography at 25° C. using an analytical size-exclusion column (TSKgel G3000 SW XL).

### Composition Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4 (11), 2411-1423. Purity and molecular weight of the proteins were analysed by CE-SDS in the presence and absence of a reducing agent using a LabChipGXII or LabChip GX Touch (Perkin Elmer) (Perkin Elmer) with or without prior treatment with rapidPNGase F according to manufacturer's protocol. Determination of the aggregate content was performed by SE-HPLC chromatography at 25° C. using analytical size-exclusion chromatography (TSKgel G3000 SW XL or UP-SW3000 columns) equilibrated in running buffer (200 mM KH₂PO₄, 250 mM KCl pH 6.2, 0.02% NaN₃).

### Mass Determination by ESI-MS

To determine the intact/deglycosylated and deglycosylated/reduced masses of purified compounds, samples were analysed by Mass Spectrometry. For compounds with a human Fc and regular Fc glycosylation, 25 μg of protein were diluted with Milli-Q Type 1 Ultrapure Water up to a volume of 40 μL. Then, 9 μL 250 mM sodium phosphate pH 7.5 and 1 μL PNGase F PRIME™ (N-Zyme Scientifics, NZPP550, Lot #NZ-2019-0123, 0.2 g/L) were added and the mix was incubated overnight at 37° C. For proteins with a non-human Fc and/or compounds containing more than the regular N-glycosites, 10 μg of protein were diluted up to 8 μL using Milli-Q Type 1 Ultrapure Water, mixed with, 2 μL “non-reducing” buffer (Rapid PNGaseF non-reducing buffer, NEB #P0711S, Lot.10085473, 10/21) and incubated for 5 minutes at 70° C. Then, 1 μL of Rapid PNGase F was added and incubated for 10 min at 50° C. After deglycosylation, samples were diluted up to 20 μL, using Milli-Q Type 1 Ultrapure Water. To reduce the cysteine bonds, 20 μL of human Fc proteins were mixed with 20 μL of freshly prepared reducing agent (7 M Guanidine, 0.4 M Tris pH 8.0, 0.5 M DTT). For the samples digested by the rapidPNGase F, 7 μL were mixed with 7 μL reducing agent. For all types of samples reduction and denaturation was performed at 70° C. for 5 min. The samples were desalted by reversed phase chromatography on a divinylbenzene column (Agilent, PLRP-S 1000 Å, 2.1×150 mm, 8 μm, 0.7 mL/min, 75° C., 1 μg on column) and mass spectra were recorded using a QTOF type mass spectrometer (Agilent 6545XT AdvanceBio LC/Q-TOF). The mass spectrometer was calibrated before each sample sequence and lock mass correction was applied to obtain high mass accuracy. For data analysis, Roche MassAnalyzer was used to sum up the mass spectra of the chromatographic peaks and to interpret detected masses. The determined masses were then compared to the calculated theoretical masses.

Alternatively, LC-MS characterization was performed at NMI Technology Transfer (Reutlingen, Germany). For determination of the intact mass 12.5-25 μg per sample were diluted 1:4 (v/v) with “non-reducing” buffer contained in the rapidPNGase F enzyme kit (Rapid PNGaseF non-reducing, NEB #P0711S, Lot. 10085472, 10/21) and denatured for 2 minutes at 80° C. Subsequently, 0.3 L rapidPNGase F (“non-reducing”) were added and compounds were deglycosylated for 10 minutes at 50° C. Thereafter, samples were diluted with double distilled water to a final volume of 31.25-62.5 μL. For determination of the mass of the reduced chains, 12.5-25 μg per sample were diluted 1:4 (v/v) “reducing” buffer contained in the rapidPNGase F enzyme kit (Rapid PNGaseF reducing, NEB #P0710S, Lot.10079163 07/21) and denatured for 2 minutes at 80° C. Subsequently, 0.3 μL rapidPNGase F (“reducing”) was added and compounds were deglycosylated for 10 minutes at 50° C. Thereafter, samples were diluted with double distilled water to a final volume of 31.25-62.5 μL. The samples were desalted by reversed phase chromatography on a C4 column (Acquity BEH300 C4, 1 mm 50 mm, 1.7 μm Charge 133380461; 150 μL/min, 75° C., 1.6 μg on column) and mass spectra were recorded using a QTOF type mass spectrometer (MAXIS, Bruker Daltonics). The mass spectrometer was calibrated prior to each sample sequence and lock mass correction was applied to obtain high mass accuracy. Data analysis was performed by summing up the mass spectra of chromatographic peaks and deconvoluting them with MaxEnt. Identity and integrity were examined by comparing the experimental masses with theoretical masses.

### Surface Plasmon Resonance

The binding behaviour of IFNG variants towards IFNGR1 and IFNGR1/2 was assessed by surface plasmon resonance (SPR). SPR experiments were performed on a Biacore 8K device at 25° C. with HBS-EP as running buffer (0.01 M HEPES pH 7.4, 0.15 M NaCl, 3 mM EDTA, 0.005% Surfactant P20, Biacore, Freiburg/Germany). To determine the binding affinities of IFNG variants to IFNG receptors, biotinylated Fc (kih)-IFNG compounds (50 nM) were captured in a flow cell of a streptavidin (SA) sensor chip (contact time 80 s, flow rate 10 μL/min). IFNG mutation series compounds were immobilised at a concentration of 25 nM, 80 s contact time and 10 μL/min. Immobilisation levels up to 450 resonance units (RU) were used. Subsequently, decreasing concentrations (800-3.13 nM) of Fc (kih)-IFNGR1 monomer or Fc (kih)-IFNGR1/2 heterodimer were injected as a second analyte with a flow rate of 30 μL/min over 120 seconds and dissociation was monitored for 500 s. Bulk refractive index differences were corrected for by subtracting the response obtained in a reference flow cell, where no protein was immobilised. The affinity constants were derived from the rate constants by fitting to a 1:1 Langmuir binding.

### HEK Blue Reporter Assay

The HEK Blue IFNG reporter cell assay allows to specifically measure the activity of IFNG. The HEK blue detection medium is used to detect the production of SEAP over time. HEK-Blue™ detection medium was prepared by pouring the contents of one pouch of HEK-Blue™ Detection in a 50 mL Falcon tube and solubilizing the powder in 50 mL of endotoxin-free water. The solution was homogenised by vortexing or swirling. The reconstituted HEK-Blue™ Detection was warmed up to 37° C. over 20-60 min. The medium was sterile filtered (0.2 μm membrane into a sterile vial/bottle). HEK-Blue™ Detection medium was kept at 37° C. before use and stored at 2-8° C. for up to 2 weeks. HEKblue cells were detached by harsh pipetting to create a homogeneous cell suspension and cell count and viability were determined using ViCell Cell Viability Analyzer. The needed amount of HEKblue cells were centrifuged and resuspended in HEKblue detection medium at 0.55 Mio/mL. 180 μL Cells (corresponding to 100 000 cells) were distributed per well according to the plate scheme and 20 μL of the test compound (diluted in PBS) were added to each well. The plate was incubated at 37° C. in a CO₂ incubator for 20-24 hours and SEAP levels over time were analysed using a spectrophotometer at 620-655 nm.

### Activity Assay

This assay was used either in combination or instead of the HEK blue reporter cell assay to assess the activity of IFNG compounds. The induction of MHC-I and PD-L1 on murine MC38-huCEA tumor cell lines was assessed in response to treatment for two days with anti-murine IFNG scFv-masked FAP-IFNG compounds containing a cleavable linker 2 (PQARK cleavage site) and compared to the activity of unmasked IgG-fused IFNG. The constructs were incubated for two hours at 37° C. with recombinant matriptase prior to treatment. The FAP-IFNG scFv-masked PQARK constructs induced MHC-I and PD-L1 in tumor cell lines when the PQARK linker was digested with Matriptase. In contrast, the FAP-IFNG scFv-masked construct without pre-incubation with recombinant matriptase did not induce MHC-I or PD-L1 upregulation.

MC38-huCEA cells were cultured in DMEM 10% FCS and harvested using a cell dissociation buffer. Cells were washed in DMEM 10% FCS, resuspended in DMEM 10% FCS, followed by assessment of cell viability and cell numbers using Eve cell counter. Cells were diluted to a concentration of 50,000/mL in DMEM 10% FCS and 100 μL of this cell suspension was seeded in cell culture treated 96F-well plates. Cells were incubated overnight at 37° C., 5% CO₂ to ensure adherence of cells. Selected concentrations of FAP-IFNG constructs were digested with or without 163 nM/4.4 ng recombinant matriptase (4735-SE, lot RIK071951, 0.44 mg/mL) at 37° C. for two hours in matriptase buffer (50 mM Tris, 50 mM NaCl, 0.01% Tween 20, pH 9.0). After incubation, digested cytokine Fc-fusion solutions were diluted with DMEM 10% FCS to a concentration of 30 nM, and 50 μL were added per well of pre-seeded cells in 100 μL DMEM 10% FCS, rendering the final maximal concentration 10 nM per well. Cytokine Fc-fusion solutions were serially diluted in a ratio 1:10 until a final minimal concentration of 0.1 pM per well. The cells were incubated for 48 hours in the incubator. After 48 hours, cells were washed with PBS, followed by a 10 minute incubation with 50 μL Trypsin EDTA. Detached cells were harvested in DMEM 10% FCS and transferred in a round bottom 96 well-plate. Cells were centrifuged (500 g, 2 min), supernatant was discarded and 150 μL PBS was added per well followed by centrifugation (500 g, 2 min). Cells were resuspended in 50 μL staining mix containing Zombie Near IR fixable Viability Dye (Invitrogen, L10119). Afterwards cells were washed with FACS buffer and 50 μL antibody staining mix containing anti-muH-2Kb/H-2D-PE (BioLegend, 114608) and anti-muCD274-APC (BioLegend, 124312) was added for 20 min at 4° C. Thereafter, cells were washed with PBS and resuspended in 100 μL PFA and incubated for 25 min at room temperature. Afterwards, cells were washed with 100 μL FACS buffer, resuspended in 100 μL FCS buffer and measured on a BD FACS Canto.

### Example 1

### 1.1 Engineering of the C-Terminus of IFNG to Modulate Homogeneity and Activity

IFNG is a key immuno-regulatory cytokine that is used to treat various immunological diseases. Its clinical application as an anti-cancer drug, however, has been limited i.a. due to the distribution of the IFNG receptor 1 (IFNGR1), which traps IFNG throughout the body and prevents its enrichment at the tumour site. Another challenge we observed is the inherent sensitivity of the IFNG C-terminus towards proteases present in production cell lines, which results in a significant amount of partially cleaved side products with truncated C-terminal sequences leading to heterogeneity of the final product (FIG. 1A). To increase compound homogeneity and stability we applied sequence engineering to the IFNG C-terminus and identified IFNG variants with differentiated side product profiles and selective functional behaviour (FIG. 1B). We initially designed a series of IFNG C-terminal deletion variants to identify the shortest C-terminal sequence of IFNG that maintained functional activity in signalling assays (Table 1A). Several C-terminal variants of IFNG were already described and highlighted the importance of the KRKR residues in the C-terminus of IFNG (FIG. 1). Deletion of the KRKR sequence (SEQ ID NO:78) renders IFNG inactive in downstream signalling (Dobeli et al., Journal of Biotechnology 1988, 7 (3), 199-216). Shortened IFNG variants, however, were shown to have similar activity as wild-type IFNG (Slodowski et al., Eur. J. Biochem. 1991, 202. 1133-1140). We therefore combined systematic shortening of the KRKR patch with Proline- or serine-proline capping to maximise C-terminal stability and sequence homogeneity in analogy to the methods described for the C-terminus of IgGs (van den Bremer et al., mAbs 2015, 7:4, 672-680). The desired candidate should show the least number of side products and a signalling activity comparable to wild-type IFNG.

TABLE 1A
The C-terminal heavy chain sequences of IFNG-fused compounds in the
deletion series. IFNG variants were linked to the C-terminus of an
IgG using 15 amino acid linkers with the sequence GGGGSGGGGSGGGGS
(SEQ ID NO: 66).
Deletion Series

| Compound | SEQ ID | C-terminal | |
|---|---|---|---|
| ID | Nos: | IFNG sequence | Description |
| P1AF3568 | 79, 41 | . . . AKTGKRKRSQMLFRG | Wild-type, reference |
| P1AF3569 | 80, 41 | . . . AKTG | Inactive variant |
| P1AF3570 | 81, 41 | . . . AKTGKRKRSQ | from Slodowski et al. 1991 |
| P1AF3571 | 82, 41 | . . . AKTGKRKRSL | from Slodowski et al. 1991 |
| P1AF3574 | 83, 41 | . . . AKTGKRKRP | Intact KRKR, P-cap |
| P1AF3575 | 84, 41 | . . . AKTGKRKP | Shortened KRKR, P-cap |
| P1AF3576 | 85, 41 | . . . AKTGKRP | Shortened KRKR, P-cap |
| P1AF3577 | 86, 41 | . . . AKTGKP | Shortened KRKR, P-cap |
| P1AF3578 | 87, 41 | . . . AKTGKRKRSP | Intact KRKR, SP-cap |
| P1AF9470 | 88, 41 | . . . AKTGKRKSP | Shortened KRKR, SP-cap |
| P1AF3580 | 89, 41 | . . . AKTGKRSP | Shortened KRKR, SP-cap |
| P1AF3581 | 90, 41 | . . . AKTGKSP | Shortened KRKR, SP-cap |

The second series was based on P1AF3574 from the deletion series and we introduced additional point mutations in the KRKR sequence with the goal to further increase proteolytic stability (Table 1B and FIG. 1B). In a systematic sequence variation approach we replaced one or two positions in the KRKR sequence with serine, proline or glutamate. Serine was chosen as a neutral amino acid, Proline was introduced with the aim to sterically interfere with proteolytic recognition sequences and Glutamate was introduced to partially reverse the charge of the KRKR sequence (SEQ ID NO:78).

TABLE 1B
The C-terminal heavy chain sequences of IFNG-fused compounds in the
deletion series. The positions refer to the KRKR sequence (SEQ ID NO:78). IFNG
variants were linked to the C-terminus of an IgG using 15 amino acid linkers with
the sequence GGGGSGGGGSGGGGS (SEQ ID NO: 66).
Mutation Series

| | SEQ ID | C-terminal IFNG | |
|---|---|---|---|
| Compound ID | Nos: | sequence | Description |
| P1AF9464 | 91, 41 | AKTGSRKRP | S in position 1 |
| P1AF9469 | 92, 41 | AKTGKSKRP | S in position 2 |
| P1AF9465 | 93, 41 | AKTGKRSRP | S in position 3 |
| P1AF9470 | 94, 41 | AKTGKRKSP | S in position 4 |
| P1AF9475 | 95, 41 | AKTGPRKRP | P in position 1 |
| P1AF9476 | 96, 41 | AKTGKPKRP | P in position 2 |
| P1AF9477 | 97, 41 | AKTGKRPRP | P in position 3 |
| P1AF9478 | 98, 41 | AKTGKRPPP | P in position 4 |
| P1AF9483 | 99, 41 | AKTGERKRP | E in position 1 |
| P1AF9484 | 100, 41 | AKTGKEKRP | E in position 2 |
| P1AF9485 | 101, 41 | AKTGKRERP | E in position 3 |
| P1AF9486 | 102, 41 | AKTGKRKEP | E in position 4 |
| P1AF9473 | 103, 41 | AKTGSRSRP | S in position 1 and 3 |
| P1AF9481 | 104, 41 | AKTGPRPRP | P in position 1 and 3 |
| P1AF9489 | 105, 41 | AKTGERERP | E in position 1 and 3 |
| P1AF9474 | 106, 41 | AKTGKSKSP | S in position 2 and 4 |
| P1AF9482 | 107, 41 | AKTGKPKPP | P in position 2 and 4 |
| P1AF9490 | 108, 41 | AKTGKEKEP | E in position 2 and 4 |
| P1AF9471 | 109, 41 | AKTGSSKRP | S in position 1 and 2 |
| P1AF9479 | 110, 41 | AKTGPPKRP | P in position 1 and 2 |
| P1AF9487 | 111, 41 | AKTGEEKRP | E in position 1 and 2 |
| P1AF9472 | 112, 41 | AKTGKRSSP | S in position 3 and 4 |
| P1AF9480 | 113, 41 | AKTGKRPPP | P in position 3 and 4 |
| P1AF9488 | 114, 41 | AKTGKREEP | E in position 3 and 4 |

### 1.2 Production and Purification of IgG-Fused IFNG Deletion and Mutation Variants

All IFNG deletion and mutation series variants were produced, purified and the impact of the engineered changes on the side product profiles and signalling activity was characterised (Example 2). IFNG sequence variants were fused via a glycine-serine linker to the C-terminus of the heavy chain of an IgG antibody specific for fibroblast activation protein (FAP), i.e. anti-FAP clone 4B9 (disclosed in WO 2012/020006 A2) and an Fc domain containing the P329G LALA mutation (Schlothauer et al., Protein Eng Des Sel. 2016, 29 (10), 457-466) (FIG. 1A). Compounds were expressed using the transient CHO expression system at evitria. The compounds of the deletion series (Table 1A) reached a median titer of 118 mg/L, compounds of the mutation series (Table 1B) were expressed with 45.5 mg/L (median value). Tables 2A and 2B list the individual titer values obtained for each compound. Next, the compounds were captured via Mab SelectSure HP and eluted with a pH gradient to pH 3.0. Fractions were neutralised and analysed for composition by CE-SDS and HMW content by SE-HPLC. Fractions with the highest monomer content were pooled and further purified by SEC. SEC Fractions with the highest monomer content were pooled as the final batch. The median monomer content as determined by SE-HPLC for the compounds of the deletion series was 88.9%, compounds of the mutation series were purified to a median 99.8% homogeneity. LC-MS characterization confirmed sequence identity for both series. The detailed LC-MS quality profiles of the compounds are provided in Example 2, Table 3. Expression titers and monomer contents for each compound are listed in Tables 2A and 2B.

TABLE 2A
Quality profiles of purified compounds of the deletion series.
Production titers were determined by Protein A SE-HPLC, monomer
content was assessed by SE-HPLC. LC-MS confirmed correct compound
identity, the detailed side product profiles are listed in Table 3.
Deletion Series

| | | SE-HPLC quality profile | | |
|---|---|---|---|---|
| Compound ID | Titer (mg/L) | % Monomer | % HMW | % LMW |
| P1AF3568 | 36.1 | 98.7 | 1.3 | 0 |
| P1AF3569 | 143.1 | 99.3 | 0.7 | 0 |
| P1AF3570 | 103.3 | 87.1 | 0.2 | 12.6 |
| P1AF3571 | 107.2 | 85.3 | 0.4 | 14.4 |
| P1AF3574 | 76.2 | 86.7 | 0.2 | 13.1 |
| P1AF3575 | 106.3 | 88.3 | 0.2 | 11.6 |
| P1AF3576 | 126.5 | 92.4 | 0.1 | 7.5 |
| P1AF3577 | 119.8 | 88.9 | 0.1 | 11 |
| P1AF3578 | 116.5 | 87.6 | 0.1 | 12.3 |
| P1AF9470 | 125.4 | 90.6 | 0.4 | 9.1 |
| P1AF3580 | 133.8 | 88.9 | 0.2 | 10.9 |
| P1AF3581 | 135.1 | 91 | 0.3 | 8.8 |

TABLE 2B
Quality profiles of purified compounds of the mutation series.
Production titers were determined by Protein A SE-HPLC, monomer
content was assessed by SE-HPLC. LC-MS confirmed correct compound
identity, the detailed side product profiles are listed in Table 3.
Mutation Series

| | | SE-HPLC quality profile | | |
|---|---|---|---|---|
| Compound ID | Titer (mg/L) | % Monomer | % HMW | % LMW |
| P1AF9464 | 32.75 | 99.7 | 0.3 | 0 |
| P1AF9469 | 24.75 | 99.7 | 0.3 | 0 |
| P1AF9465 | 24.75 | 99.7 | 0.3 | 0 |
| P1AF9470 | 32.5 | 99.8 | 0.2 | 0 |
| P1AF9475 | 38 | 99.8 | 0.2 | 0 |
| P1AF9476 | 39 | 99.7 | 0.3 | 0 |
| P1AF9477 | 38.75 | 99.8 | 0.2 | 0 |
| P1AF9478 | 16 | 99.2 | 0.8 | 0 |
| P1AF9483 | 56.1 | 99.8 | 0.2 | 0 |
| P1AF9484 | 56.7 | 99.8 | 0.2 | 0 |
| P1AF9485 | 55.6 | 99.8 | 0.2 | 0 |
| P1AF9486 | 55.6 | 99.8 | 0.2 | 0 |
| P1AF9473 | 33 | 99.6 | 0.4 | 0 |
| P1AF9481 | 51.9 | 99.8 | 0.2 | 0 |
| P1AF9489 | 65.1 | 99.8 | 0.2 | 0 |
| P1AF9474 | 36.75 | 99.7 | 0.3 | 0 |
| P1AF9482 | 57.9 | 99.8 | 0.2 | 0 |
| P1AF9490 | 96 | 99.9 | 0.1 | 0 |
| P1AF9471 | 35.75 | 99.7 | 0.3 | 0 |
| P1AF9479 | 57.4 | 99.8 | 0.2 | 0 |
| P1AF9487 | 67.4 | 99.8 | 0.2 | 0 |
| P1AF9472 | 35.75 | 99.7 | 0.3 | 0 |
| P1AF9480 | 57.3 | 99.8 | 0.2 | 0 |
| P1AF9488 | 62.3 | 99.8 | 0.2 | 0 |

### Example 2

2.1 Identification of C-Terminal Variants of IFNG with Improved Quality Profiles

To maximise sequence integrity of the C-terminus of IFNG, we identified an IFNG sequence with a favourable heavy chain side product profile. Therefore, we combined proline- or serine-proline capping with systematic shortening of the KRKR sequence (FIG. 1B, Table 1A). All compounds were expressed, purified and final batches were analysed by LC-MS to identify and quantify heavy chain side products (Table 3). The compound comprising wild-type human IFNG contained 7% intact heavy chains and 8 truncated heavy chain side products in varying quantities. The published IFNG variants (as included in P1AF3570 and P1AF3571) also displayed heavy chain sequence heterogeneity due to proteolysis with 3 and 4 side products, respectively. The number of side products correlated with the length of the IFNG C-terminal sequence: shortening of the C-terminus reduced the number of truncated heavy chains and compounds without the KRKR sequence were 100% correct. C-terminal proline-capping resulted in less heavy chain side products compared to serine-proline capped equivalents (Table 3).

TABLE 3
Side product profiles of IFNG benchmark molecules (P1AF3570, P1AF3571) and deletion
series molecules with different C-terminal sequences. The bold sequence for each
compound corresponds to the C-terminal full-length sequence and observed truncation
products are listed subsequently. Mass peak intensities were used to estimate the
relative amount (%) of each side product.

| Compound ID | C-terminal HC sequence | Relative amount (%) |
|---|---|---|
| P1AF3568 | . . . AKTGKRKRSQMLFRG | 7 |
| | . . . AKTGKRKRSQMLFR | 15 |
| | . . . AKTGKRKRSQMLF | 14 |
| | . . . AKTGKRKRSQML | 29 |
| | . . . AKTGKRKRSQM | 5 |
| | . . . AKTGKRK | 16 |
| | . . . AKTGKR | 1 |
| | . . . AKTGK | 11 |
| | . . . AKTG | 1 |
| P1AF3569 | . . . AKTG | 100 |
| P1AF3570 | . . . AKTGKRKRSQ | 76 |
| | . . . AKTGKRK | 10 |
| | . . . AKTGK | 11 |
| | . . . AKTG | 3 |
| P1AF3571 | . . . AKTGKRKRSL | 3 |
| | . . . AKTGKRK | 54 |
| | . . . AKTGKR | 5 |
| | . . . AKTGK | 33 |
| | . . . AKTG | 5 |
| P1AF3574 | . . . AKTGKRKRP | 95 |
| | . . . AKTGK | 5 |
| P1AF3575 | . . . AKTGKRKP | 100 |
| P1AF3576 | . . . AKTGKRP | 100 |
| P1AF3577 | . . . AKTGKP | 100 |
| P1AF3578 | . . . AKTGKRKRSP | 60 |
| | . . . AKTGKRK | 20 |
| | . . . AKTGK | 17 |
| | . . . AKTG | 3 |
| P1AF9470 | . . . AKTGKRKSP | 98 |
| | . . . AKTGK | 2 |
| P1AF3580 | . . . AKTGKRSP | 100 |
| P1AF3581 | . . . AKTGKSP | 100 |

### 2.2 Signalling Activities of IFNG Deletion Variants

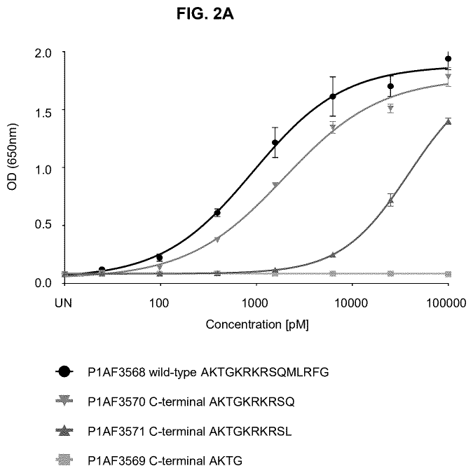

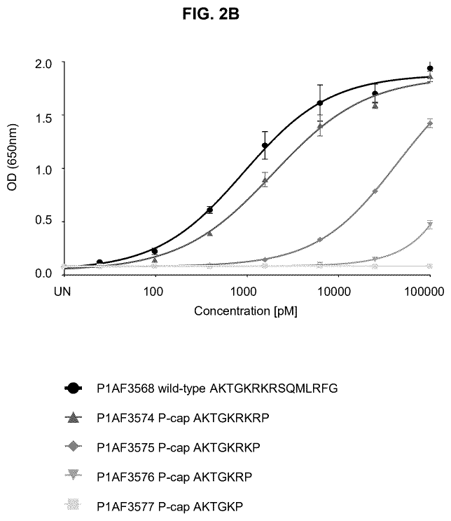

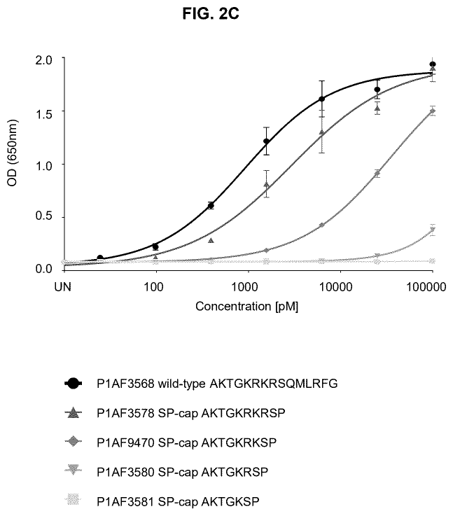

Benchmark compounds and other compounds of the deletion series were further characterised for their ability to induce IFNG signalling using the HEKblue assay. Functional activity correlated with the length of the C-terminal sequence: only compounds comprising the entire KRKR sequence retained activity levels comparable to wild-type IFNG (FIG. 2 and Table 4), while systematic shortening of the KRKR sequence gradually reduced functional activity.

TABLE 4
Signalling activity (area under the curve)
relative to wild-type IgG-IFNG as
determined in the HEKblue assay

| | C-terminal | Relative |
|---|---|---|
| Compound ID | HC sequence | activity |
| P1AF3568 | . . . AKTGKRKRSQ | 100% |
| wild-type | MLFRG | |
| P1AF3569 | . . . AKTG | 9% |
| P1AF3570 | . . . AKTGKRKRSQ | 81% |
| P1AF3571 | . . . AKTGKRKRSL | 32% |
| P1AF3574 | . . . AKTGKRKRP | 84% |
| P1AF3575 | . . . AKTGKRKP | 35% |
| P1AF3576 | . . . AKTGKRP | 13% |
| P1AF3577 | . . . AKTGKP | 9% |
| P1AF3578 | . . . AKTGKRKRSP | 79% |
| P1AF9470 | . . . AKTGKRKSP | 40% |
| P1AF3580 | . . . AKTGKRSP | 12% |
| P1AF3581 | . . . AKTGKSP | 9% |

Combined with the quality data (Table 3), the best profile, i.e. the highest activity combined with least amounts of side products, was observed for the proline-capped IFNG variant terminating in KRKRP (P1AF3574). The compound contained 95% correct heavy chains, 5% truncated side products and showed 84% activity compared to wild-type IFNG. Consequently, this sequence was chosen as the basis for further stability engineering in the mutation series (Example 2.4).

### 2.3 IFNG-KRKRP and Wild-Type IFNG Show Comparable Binding Behaviour Towards IFNG Receptors

IFNG is a constitutive homodimer which initiates the formation of a signalling complex with the IFNG receptors to activate the JAK/STAT signalling pathway. The signalling complex consists of a symmetric arrangement of two IFNGR1/2 heterodimers with the IFNG homodimer at the centre. Each IFNG monomer interacts with one IFNGR1/2 heterodimer making contacts with both receptor subunits. Signalling complex assembly is suggested to be triggered by an initial interaction of IFNG with IFNGR1, which creates the binding surface for IFNGR2 (Mendoza et al., Nature 2019, 567 (7746), 56-60). While signalling complex assembly is mainly driven by the structured domain of IFNG, the unstructured C-terminus—of IFNG also contributes to active signalling likely by interacting with sites in IFNGR1/2 closer to the cell membrane. The exact binding mode, however, is unknown and not resolved in available crystal structures.

To further evaluate analogous properties of wild-type IFNG and our engineered compound with C-terminal KRKRP (SEQ ID NO:1), we determined the binding affinities of IFNG variants towards IFNG receptors by SPR. Therefore, wild-type IFNG (P1AG2651) and IFNG KRKRP (P1AG1550) were produced and purified as C-terminal Fc (kih)-fusion proteins and immobilised in the flow cell. The knob-into-holes (kih) mutations are specific mutations in the Fc domain (mutations S354C and T366W EU numbering in the “knob” chain and mutations Y349C, T366S, L368A and Y410V EU numbering in the “hole” chain) that allow enhanced heterodimerization of two different heavy chains (Merchant et al., Nature Biotechnology 1998, 16 (7), 677-681). As second analytes, either IFNGR1 monomer (P1AF7104) or IFNGR1/2 heterodimer (P1AF8126) were passed through the flow cell at different concentrations and dissociation constants (KD) were calculated based on observed association and dissociation behaviour. The Fc (kih)-IFNG KRKRP bound IFNGR1 with an apparent K_{D} of 154 nM, which was in a similar range as wild-type Fc (kih)-IFNG with K_{D} of 177 nM. For the IFNGR1/2 heterodimer, higher apparent binding affinities were observed: Fc (kih)-IFNG-KRKRP (P1AG1550) bound with a K_{D} of 54.4 nM, wild-type Fc (kih)-IFNG (P1AF2651) bound with K_{D} of 73.8 nM.

### 2.4 Mutations of the IFNG KRKR Sequence Differentially Affect IFNG Receptor Binding and Signalling

Having identified the minimal C-terminal sequence length, we introduced additional point mutations in the KRKR sequence to further improve stability towards proteases. All compounds of the mutation series were expressed and purified as IgG-fusion proteins (see Example 1). Analysis of the quality profile by LC-MS confirmed the beneficial effect of the C-terminal proline-cap as proteolytic side products were not detected in the compounds of this mutation series.

Mutations of the KRKR sequence had an effect on receptor binding as determined by SPR. To measure binding affinities, compounds of the mutation series were immobilised in the flow cell. As a second analyte Fc (kih)-IFNGR1 monomer or Fc (kih)-IFNGR1/R2 heterodimer were passed through the flow cell and binding affinities (KD) were calculated based on observed association and dissociation constants. Already single point mutations reduced the apparent binding affinities towards the receptors compared to the compound with C-terminal KRKRP. Double point mutations had an additional negative effect on receptor binding. The binding behaviour of compounds of the mutation series towards IFNGR1 and IFNGR1/2 is summarised in Table 5.

TABLE 5
Apparent binding affinities of compounds
of the mutation series towards Fc(kih)-IFNGR1
monomer and Fc(kih)-IFNGR1/2 heterodimer

| Compound | | Kd IFNGR1 | Kd IFNGR1/R2 |
|---|---|---|---|
| ID | C-terminus | (nM) | (nM) |
| P1AG1310 | KRKRP | 475 | 167 |
| PIAF9464 | SRKRP | 614 | 459 |
| P1AF9475 | PRKRP | 643 | 320 |
| P1AF9483 | EKKRP | 3160 | 1080 |
| P1AF9469 | KSKRP | 708 | 652 |
| P1AF9476 | KPKRP | 1020 | 456 |
| P1AF9484 | KEKRP | 3260 | 1150 |
| P1AF9465 | KRSRP | 597 | 431 |
| P1AF9477 | KRPRP | 539 | 312 |
| P1AF9485 | KRERP | 1200 | 608 |
| P1AF9470 | KRKSP | 604 | 535 |
| P1AF9478 | KRKPP | 564 | 284 |
| P1AF9486 | KRKEP | 1370 | 598 |
| P1AF9471 | SSKRP | 1730 | 1420 |
| P1AF9479 | PPKRP | 3600 | 204 |
| P1AF9487 | EEKRP | 1030000 | 1750 |
| P1AF9472 | KRSSP | 1080 | 885 |
| P1AF9480 | KRPPP | 1420 | 683 |
| P1AF9488 | KREEP | 6220 | 2870 |
| P1AF9473 | SRSRP | 1100 | 793 |
| P1AF9481 | PRPRP | 977 | 486 |
| P1AF9489 | ERERP | 10700 | 5960 |
| P1AF9474 | KSKSP | 4560 | 864 |
| P1AF9482 | KPKPP | 2710 | 950 |
| P1AF9490 | KEKEP | 216000 | 4750 |

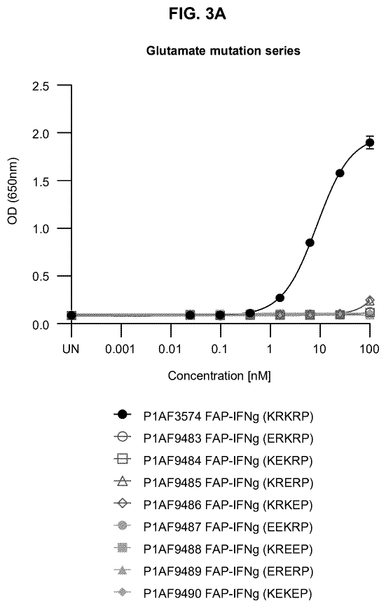

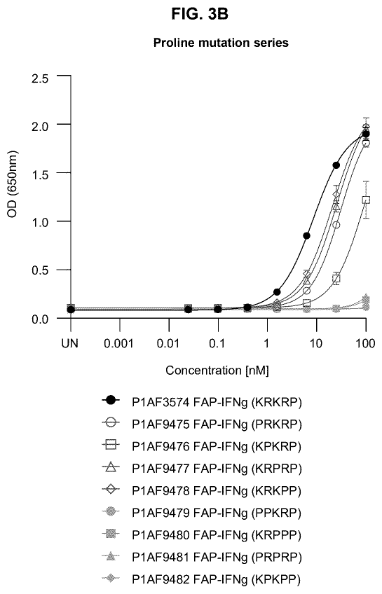

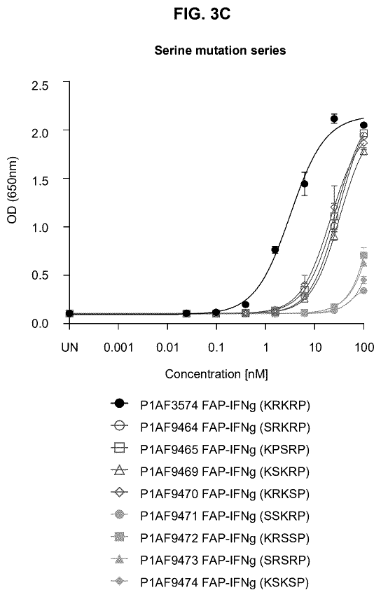

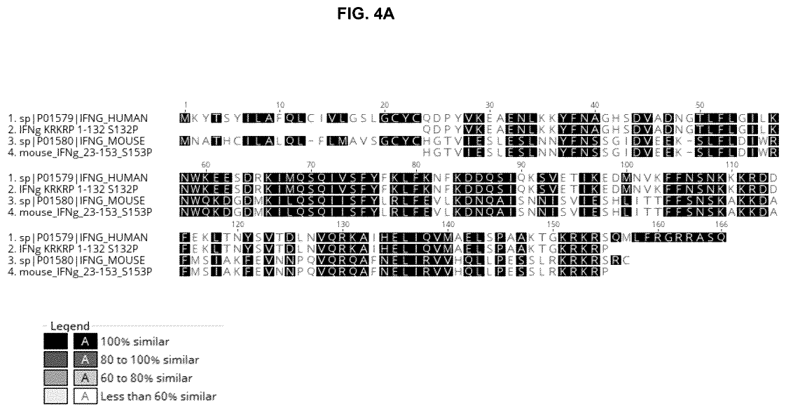

Impaired functionality of mutation series variants was even more apparent in the reporter cell assay (FIG. 3). Functional characterization revealed that either single serine or proline mutations were well tolerated irrespective of their position in the KRKR sequence. In contrast, already single glutamate mutations rendered the compound inactive, as did all double mutations irrespective of the exchanged amino acids.

2.5 Mutations in the IFNG KRKR Sequence Protect from Proteolysis by Matriptase

To assess the effect of mutations on the proteolytic stability of the KRKR motif, all compounds of the mutation series were treated with matriptase (custom purification according to published protocols by Cepter Biopartners) at a molar enzyme to substrate ratio of 1:3500 for 1 h at 37° C. Subsequently, the release of the main proteolytic side product (IgG-IFNG cleaved between IFNG-K/RKRP) was detected and quantified by LC-MS in the deglycosylated-reduced state. Point mutations of positions 1 or 2 had a positive effect on proteolytic stability with no observed proteolysis at the chosen conditions, while for the reference molecule 35% cleavage of the K/RKR motiv was detected (Table 6). Serine, proline or glutamate mutations in position 3 or 4 did interfere with proteolysis less efficiently and 2-11% proteolytic products were detected. Double mutations that included position 1 or 2 also efficiently prevented proteolysis, while double mutations in positions 3 and 4 were permissive. Nevertheless, given the lower functional activity of mutation series compounds in absence of target binding, IFNG KRKRP remained the preferred sequence and was used to create masked IFNG compounds (Example 3).

TABLE 6
Mutation of position 1 and 2 in the KRKR sequence
motif improves proteolytic stability of the KRKR patch.
Compounds were incubated with matriptase and proteolysis
profiles were analysed and quantified by LC-MS.
Positions refer to the KRKR sequence (SEQ ID NO: X)

| | Degree of K/RKR |
|---|---|
| Mutation type | proteolysis after 1 hour |
| IFNG- KRKRP | 35% (reference molecule) |
| S/P/E in position 1 | 0%/0%/0% |
| S/P/E in position 2 | 2%/0%/0% |
| S/P/E in position 3 | 6%/9%/3% |
| S/P/E in position 4 | 5%/11%/2% |
| SS/PP/EE in position 1 and 2 | 0%/0%/0% |
| SS/PP/EE in position 3 and 4 | 4%/1%/0% |
| SS/PP/EE in position 1 and 3 | 0%/0%/0% |
| SS/PP/EE in position 2 and 4 | 0%/0%/0% |

### Example 3

### 3.1 Design of Tumor-Targeted Masked IFNG Compounds

To develop an IFNG with greater therapeutic potential, we designed tumor-targeted IFNG compounds where binding of IFNG to IFNGR1 was prevented by IFNG-specific masking domains. We hypothesised that efficient masking would counteract the associated sink effect and allow for the enrichment of the compound at the tumor site to ultimately elicit the desired downstream effects. Masking domains were based on IFNG-specific scFv domains and were introduced either C-terminally (mask-release format) or N-terminally (cytokine-release format) of IFNG-KRKRP (FIG. 1C). Masked compounds were designed to be activated in the tumor microenvironment by proteolysis of a cleavable linker connecting IFNG and its masks.

To create the mask-release format, IFNG-KRKRP was fused via a glycine-serine-linker (linker 1, GGGGSGGGGSGGGGSGGGGSGGGGSGGGGG, SEQ ID NO:69) to the C-terminus of a targeting antibody which is an IgG antibody specific for fibroblast activation protein (FAP), i.e. anti-FAP clone 4B9 (disclosed in WO 2012/020006 A2) and an Fc domain containing the P329G LALA mutation (Schlothauer et al., Protein Eng Des Sel. 2016, 29 (10), 457-466) A anti-human IFNG scFv-mask (based on U.S. Pat. No. 6,329,511 B1) was fused C-terminally of IFNG-KRKRP via a second glycine-serine linker (linker 2, GGGGSGGGGSGGGGSGGGGSGGGGSGGGGG, SEQ ID NO:69). To create the cytokine-release format, the scFv mask was fused to the C-terminus of the targeting IgG antibody via a glycine-serine linker (linker 1) and IFNG-KRKRP was connected C-terminally via a second glycine-serine linker (linker 2). To enable proteolytic activation of the masked compound in the tumor microenvironment, a matriptase cleavage site (PQAR/K, SEQ ID NO:20) was introduced in linker 2 in both formats. This cleavable linker has the amino acid sequence

(linker 2′, PQARK linker, SEQ ID NO: 70)
GGGGSGGGGSGGGPQARKGGGGGGSGGGGG.

Proteolysis of linker 2′ in mask-release format yields a IgG-fused, unmasked IFNG, while proteolysis of linker 2′ in cytokine-release format yields a free IFNG-KRKRP homodimer.

TABLE 7A
Identifiers of tumor-targeted masked IFNG compounds and their formats

| | | | SEQ ID |
|---|---|---|---|
| Identifier | Format | Structure | NOs: |
| P1AG7568 | Mask release with | FAP 4B9 IgG - linker 1 - IFNG | 40, 41 |
| | cleavable linker | KRKRP - PQARK linker - scFv mask | |
| P1AG7571 | Cytokine release with | FAP 4B9 IgG - linker 1 - scFv mask - | 42, 41 |
| | cleavable linker | PQARK linker - IFNG KRKRP | |
| P1AG7567 | Mask release with non- | FAP 4B9 IgG - linker 1 - IFNG | 115, 41 |
| | cleavable linker | KRKRP - linker 2- scFv mask | |
| P1AG7570 | Cytokine release with | FAP 4B9 IgG - linker 1 - scFv mask- | 116, 41 |
| | non-cleavable linker | linker 2 - IFNG KRKRP | |
| P1AG1310 | unmasked | FAP 4B9 IgG - linker 1 - IFNG | 117, 41 |
| | | KRKRP | |

### 3.2 Production and Purification of Mask-Release and Cytokine-Release Formats

Masked formats with cleavable (P1AG7568, P1AG7571) and non-cleavable (P1AG7567, P1AG7570) linker sequences were expressed and purified using the transient expression system at WuXi Biologics. The unmasked, positive control compound (P1AG1310) was expressed at evitria AG and purified in house. All compounds were captured via MabSelectSure HP and eluted with a pH gradient to pH 3.0. Fractions were neutralised and analysed for composition by CE-SDS and HMW content by SE-HPLC. Eluted fractions with the highest monomer content were pooled and further purified by SEC. SEC fractions with the highest monomer content were pooled as the final batch (Table 7B). Depending on the format, different expression titers and quality profiles were reached: the highest production titer was observed for the unmasked compound (152 mg/L). Masked formats were expressed at lower levels (6.9-54.6 mg/L). LC-MS confirmed correct chain composition and no proteolytic side products were detected.

TABLE 7B
Quality profiles of purified masked compounds and control
molecules. Production titers were determined by Protein
A SE-HPLC, monomer content was assessed by SE-HPLC and
CE-SDS. LC-MS confirmed correct compound identity.

| | | | | | CE-SDS |
|---|---|---|---|---|---|
| Compound | Titer | SE-HPLC | | | % Main |
| ID | (mg/L) | % Monomer | % HMW | % LMW | Peak |
| P1AG7568 | 6.9 | 97.3 | 2.7 | 0 | 97.8 |
| P1AG7571 | 20 | 99.32 | 0.68 | 0 | 100 |
| P1AG7567 | 17.7 | 96.4 | 3.6 | 0 | 100 |
| P1AG7570 | 54.6 | 98.44 | 1.55 | 0 | 100 |
| P1AG1310 | 152 | 99.8 | 0.2 | 0 | 100 |

### Example 4

### 4.1 Generation of Masked Murine IFNG Formats as Murine Surrogates

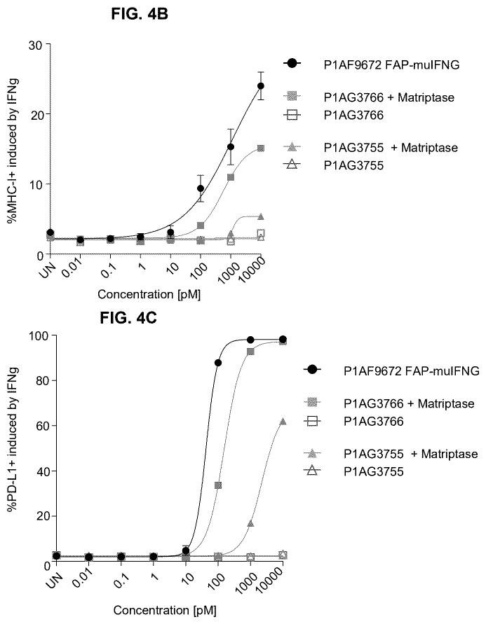

To assess the therapeutic potential of masked formats in vivo, we turned to a murine surrogate model (Example 5). Masking domains were based on scFv domains specific for murine IFNG and masks were introduced either C-terminally (mask-release format) or N-terminally (cytokine-release format) of murine IFNG-KRKRP (FIG. 1, FIG. 4A). As for the human counterparts, masked murine compounds were designed to be activated in the tumor microenvironment by proteolysis of a cleavable linker connecting IFNG and its masks.

Surrogate molecules were designed analogous to the human masked compounds: to create the mask-release format (P1AG3766), murine IFNG-KRKRP was fused via a glycine-serine-linker (linker 1, GGGGSGGGGSGGGGSGGGGSGGGGSGGGGG, SEQ ID NO:69) to the C-terminus of a murine IgG1 heavy chain of a targeting IgG antibody specific for fibroblast activation protein (FAP), i.e. anti-FAP clone 28H1 (disclosed in WO 2012/020006 A2). DAPG mutations were introduced in the Fc constant regions of the heavy chains to abrogate binding to mouse Fc gamma receptors according to the method described e.g. in Baudino et al. J. Immunol. (2008), 181, 6664-6669, or in WO 2016/030350 A1. A anti murine IFNG scFv mask was fused C-terminally of murine IFNG-KRKRP via a second glycine-serine linker (linker 2, GGGGSGGGGSGGGGSGGGGSGGGGSGGGG, SEQ ID NO:69). To create the cytokine-release format (P1AG3755), the scFv mask was fused to the C-terminus of the targeting IgG via a glycine-serine linker (linker 1) and murine IFNG-KRKRP was connected C-terminally via a second glycine-serine linker (linker 2). To enable proteolytic activation in the tumor microenvironment, a matriptase cleavage site (PQAR/K) was introduced in linker 2 of both formats (linker 2′). The cleavable linker has the amino acid sequence

(linker 2′, PQARK linker, SEQ ID NO: 70)
GGGGSGGGGSGGGPQARKGGGGGGSGGGGG.

Proteolysis of linker 2′ in mask-release format yields a IgG-fused, unmasked murine IFNG-KRKRP, while proteolysis of linker 2′ in cytokine-release format yields a free murine IFNG-KRKRP homodimer. As a positive control murine IFNG-KRKRP was also expressed as a C-terminal IgG fusion without a masking domain (P1AF9672).

TABLE 8A
Identifiers of tumor-targeted masked murine IFNG compounds and their formats

| Identifier | Format | Structure | SEQ ID NOs: |
|---|---|---|---|
| P1AG3766 | Mask release with | FAP 28H1 muIgG - linker 1 - muIFNG | 43, 44 |
| | cleavable linker | KRKRP - PQARK linker 2 - scFv mask | |
| P1AG3755 | Cytokine release | FAP 28H1 muIgG - linker 1 - scFv mask - | 45, 44 |
| | with cleavable linker | PQARK linker 2 - muIFNG KRKRP | |
| P1AG9672 | Mask release with | FAP 28H1 muIgG - linker 1 - muIFNG | 118, 44 |
| | non-cleavable linker | KRKRP | |

### 4.2 Production and Purification of Murine Surrogates

All murine surrogate compounds were expressed using the transient CHO expression system at evitria. Immediately after harvest complete protease inhibitor cocktail (Roche) was added at a concentration of 0.5× and compounds were captured using Mab SelectSure HP and eluted with a pH gradient to pH 3.0. Fractions were neutralised and analysed for composition by CE-SDS and HMW content by SE-HPLC. Fractions with the highest monomer content were pooled and further purified by SEC. After each SEC purification, respective peak fractions were analysed by SE-HPLC and CE-SDS. SEC fractions with the highest monomer content were pooled as the final batch. SE-HPLC revealed monomer contents >95% for both formats (P1AG3766 and P1AG3755) as well as the unmasked control (P1AF9672), CE-SDS and LC-MS confirmed correct chain composition (Table 8B). For the cytokine-release format (P1AG3755) LC-MS revealed that in 10% of the compounds one out of two cleavable linkers was already cleaved at the intended cleavage site (PQAR/K, SEQ ID NO:20).

TABLE 8B
Quality profiles of purified murine surrogate compounds.
Production titers were determined by Protein A SE-HPLC,
monomer content was assessed by SE-HPLC and CE- SDS.
LC-MS confirmed correct compound identity.

| | | | | | CE-SDS |
|---|---|---|---|---|---|
| Compound | Titer | SE-HPLC | | | % Main |
| ID | (mg/L) | % Monomer | % HMW | % LMW | Peak |
| P1AG3766 | 19.7 | 95.1 | 4.8 | 0 | 96.6 |
| P1AG3755 | 20.1 | 96.8 | 3.1 | 0 | 92.0 |
| P1AG9672 | 94 | 98.8 | 1.2 | 0 | 81.2 |

### 4.3 Functional Characterization of Murine Surrogates

To assess in vitro activity of the FAP-targeted scFv-masked FAP-anti-murine IFNG compounds containing a cleavable linker, MC38-huCEA cells were treated in vitro with aforementioned surrogate molecules. Anti-murine IFNG scFv-masked FAP-muIFNG compounds were incubated in the absence or presence of 163 nM recombinant murine matriptase for 2 hours at 37° C. After a 48 hours culture period, MHC-I and PD-L1 expression on MC38-huCEA cells was quantified using flow cytometry. Treatment with unmasked FAP-muIFNG compound served as a positive control demonstrating that MHC-I and PD-L1 expression on MC38-huCEA cells were induced upon treatment (FIGS. 4A and 4B).

Anti-murine IFNG scFv-masked FAP-muIFNG molecules that were not pre-treated with recombinant murine matriptase did not lead to enhanced MHC-I nor PD-L1 levels (FIGS. 4A and 4B). Pre-incubation of anti-murine IFNG scFv-masked FAP-muIFNG with Matriptase partially recovered activity as demonstrated by increased expression of MHC-I and PD-L1 on MC38-huCEA cells (FIGS. 4A and 4B).

### Example 5

### 5.1 In Vivo Characterization of Murine Surrogate Molecules

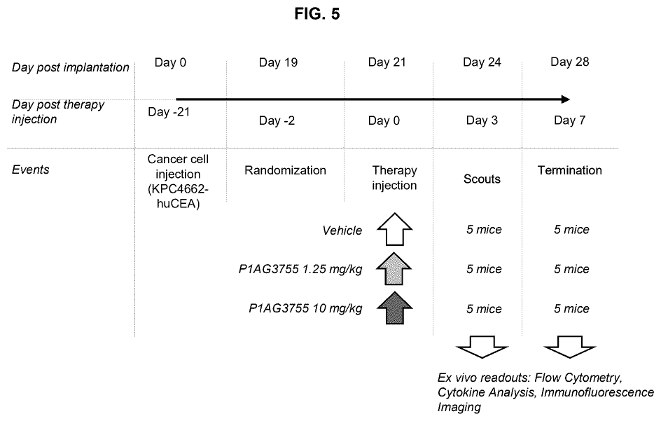

For the in vivo experiment one dose of 1.25 or 10 mg/kg of P1AG3755 was injected once i.v. into C57Bl/6 mice expressing human CEA, bearing KPC-4662 huCEA tumors (excluded tumor model with low CD8 T cell infiltration) that were injected as a cell suspension subcutaneously 21 days before the start of the treatment (FIG. 5).

KPC 4662 cells were obtained from the University of Pennsylvania and engineered in-house in order to express human CEA. Cells were cultured in DMEM+FBS 10%+Hygromycin 500 μg/ml, counted and 300,000 cells were injected in total volume of 100 μl, in a 1:1 mix with RPMI and Matrigel, subcutaneously in the flank of mice. Tumor growth was measured at least twice weekly using a caliper and tumor volume was calculated as follows:

$$Tumor volume = ( {W}^{2} / 2 ) \times L ( W : Width , L : Length ) .$$

After 21 days of initial tumour growth mice, when tumour size reached approximately 225 mm³, mice were randomised into three groups and treated three times per week with P1AG3755 at different doses or Histidine buffer (Vehicle). All mice were injected i.v. with 200 μL of the appropriate solution. To obtain the proper amount of compounds per 200 μL, the stock solutions were diluted with Histidine buffer (20 mM Histidine, 140 mM NaCl pH 6.0). On days 3 and 7 post therapy injection at least five mice were sacrificed (day 7=termination). Of these mice tumours and blood was taken for downstream analysis.

### 5.2 Ex Vivo Readouts

Tumor halves dedicated for flow cytometry analysis were weighed and digested with Liberase and DNAse. Single cell suspensions were then stained with among others DAPI, Podoplanin PE-Cy7, E-cadherin BV421, CD45 AF700, TCRb PE-Cy5 and CD8a BV711 and analysed on a BD Fortessa Flow Cytometer. Plotting and statistical testing was done in GraphPad PRISM 8.

For histological analysis by immunofluorescence (3DIP) tumor halves were fixed in BD Cytofix solution, diluted 1:4 in PBS, for ca. 20 hours. After washing and transferring to PBS, tumors were embedded in 4% low-gelling temperature agarose. Tumor sections (70 μm thick) were cut from these blocks using a Leica VT1200s Vibratome equipped with common razor blades. Subsequently sections were permeabilized (TBS+0.3% Triton-X) and blocked using BSA and Mouse Serum (each 1%) for two hours before being stained overnight (ca. 15 hours) at room temperature using among others the following antibodies: CD8a BV421, E cadherin CF488, MHCI PE. Image acquisition was done on a Leica SP8 inverted confocal microscope.

For cytokine analysis serum samples from 5 mice per group were collected (again on days 3 and 7 post therapy injection) and stored at −20° C. until needed for the assay. A piece of tumour from 5 mice per group was collected in liquid nitrogen at the same dates and stored at −80° C. until further use. 5 μL of serum ( 1/10 dilution) and 40 μg of protein from the tumour lysate were used to perform the assay. The tumor lysate was obtained using the Bio-plex cell lysis kit (171304012, BioRad) and the Precellys evolution homogenizer lysing device (Bertin instruments). The tumor lysate was centrifuged and the supernatant kept and later stored at −80° C. The protein level was determined using the Pierce BCA protein assay kit (Thermo Fisher Scientific) and an ELISA reader (PerkinElmer, EnVision 2104 Multilabel reader). The tumor lysate was diluted with the sample diluent from the kit accordingly to obtain a final amount of 40 μg of protein per sample. The assay was performed with the ProcartaPlex Simplex Kit (ref. EPX010-26061-901, Thermo Fisher) on the Luminex Flexmap 3D instrument following the manufacturer's protocol.

### Statistical Analysis

For flow cytometry and cytokine analysis One-Way Anova tests with multiple comparison analysis of treatment versus vehicle groups were run with a Dunnet correction for multiple testing. Plotting and statistical testing was done in GraphPad PRISM 8.

### 5.3 Observations

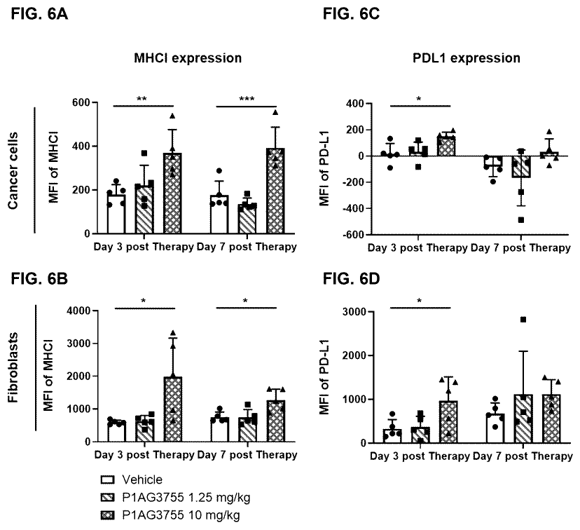

A proximal biomarker of IFNG signalling is the upregulation of MHCI and PDL1 expression. Using the murine surrogate molecules to treat KPC4662-huCEA tumors (FIG. 5), we could readily observe MHCI and PDL1 upregulation by flow cytometry (FIG. 6) 3 days after therapy administration, in the higher dose group on cancer cells and fibroblasts. Increased MHCI expression was also observable 7 days post therapy administration, while PDL 1 levels were not statistically significantly different to the vehicle group at 7 days post therapy administration.

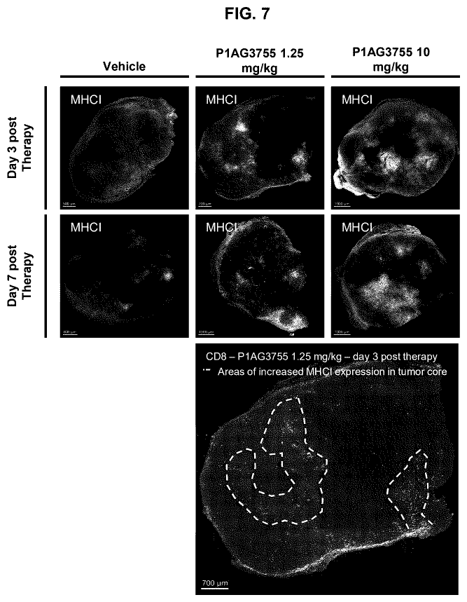

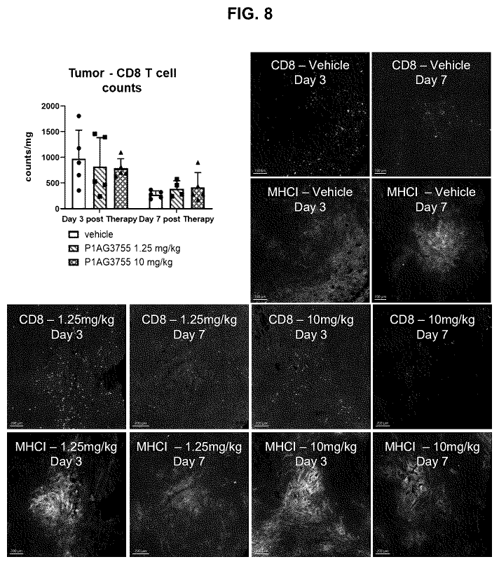

Next we observed, via immunofluorescence microscopy, that increased MHCI expression in the tumour tissue is very heterogeneous and that CD8 T cell presence is correlated with MHCI positive areas in the tumour tissue (FIG. 7).

While overall CD8 T cell presence was not statistically significantly different between treatment and Vehicle groups by flow cytometry, we again showed via imaging that especially early after treatment the amount of CD8 T cells that are present in the MHCI expressing regions of the tumors are increased (FIG. 8). As in this tumor CD8 T cells are also present at baseline and in the Vehicle group at the edges of the tumor, the non-localized quantification via Flow Cytometry was not able to pick up a statistically significant difference between treatment groups and Vehicle.

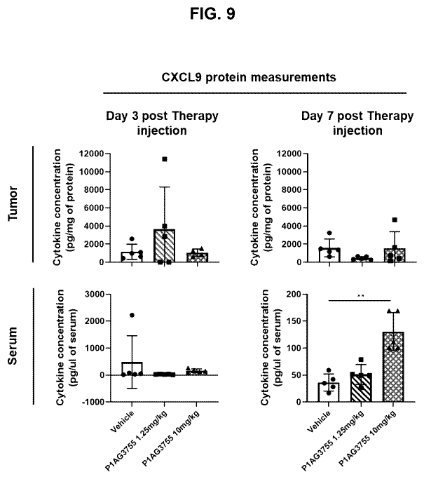

Lastly, we could observe that treatment with the murine surrogates led to an increased CXCL9 (T cell attractant chemokine) level 7 days post therapy administration in the serum of the mice (FIG. 9), showing that not only the cancer cells change and the tumor immune environment changes, but also the cytokine milieu is impacted by tumor-targeted IFNG.

### Example 6

### 6.1 Design of Sterically Masked IFNG

Given the species specificity of IFNG, also species specific masking domains are required to prevent receptor binding outside of the tumor microenvironment. We therefore explored species-independent masking and designed a series of sterically masked, targeted IFNG compounds. Based on the available crystal structure of the IFNG signalling complex (Mendoza et al., Nature 2019, 567 (7746), 56-60), we hypothesised that in the assembled complex the C-termini of IFNG are located close to the membrane and that fusion of a bulky domain to the C-terminus could therefore interfere with IFNGR1 binding. To create a targeted, masked molecule we inserted the IFNG domain between the upper and lower hinge region of IgG1. N- and C-terminally, IFNG was connected using glycine-serine linkers. Additionally we explored several hinge variants.

TABLE 9A
Identifiers of tumor-targeted masked murine
IFNG compounds and their formats

| | SEQ ID | |
|---|---|---|
| Identifier | Nos: | Structure |
| P1AG8692 | 119, 41 | Fab - linker 1 - IFNG KRKRP - |
| | | linker 2 - hinge 1 - Fc |
| P1AG8693 | 120, 41 | Fab - linker 1 - IFNG KRKRP - |
| | | linker 3 - hinge 1 - Fc |
| P1AG8694 | 121, 41 | Fab - linker 1 - IFNG KRKRP - |
| | | linker 3 - hinge 2 - Fc |
| P1AG8695 | 122, 41 | Fab - linker 1 - IFNG KRKRP - |
| | | linker 3 - Fc |
| P1AG8696 | 123, 41 | Fab - linker 1 - IFNG KRKRP - |
| | | linker 5 - hinge 1 - Fc |
| P1AG8697 | 124, 41 | Fab - linker 4 - IFNG KRKRP |

The amino acid sequences of the different linkers are as follows: linker 1 is GGGGSGGGGSGGGGSGGGGSGGGGSGGGGG (SEQ ID NO:69), linker 2 is GGGGSGGG (SEQ ID NO:72), linker 3 is LEVLFQGP (SEQ ID NO:73), linker 4 is GGGGSGGGGSGGGGSGGGGSGLEVLFQGPGGGGGSGGGGG (SEQ ID NO:74) and linker 5 is QARK (SEQ ID NO:75). The amino acid sequence of hinge 1 is TCPPCP (SEQ ID NO: 76), hinge 2 is PCP (SEQ ID NO:77).

### 6.2 Production and Purification of Sterically Masked IFNG

Sterically masked compounds were expressed using the transient CHO expression system at evitria, captured via MabSelectSure HP and eluted with a pH gradient to pH 3.0. Fractions were neutralised and analysed for composition by CE-SDS and HMW content by SE-HPLC. Fractions with the highest monomer content were pooled and further purified by SEC. In some cases a second and third SEC step was required to remove all HMW impurities. After each SEC purification, respective peak fractions were analysed by SE-HPLC and CE-SDS. Fractions with the highest monomer content were pooled as the final batch. The median monomer content was >98% for all compounds (SE-HPLC, 100% by CE-SDS) and sequence identity was confirmed by LC-MS. Detailed quality attributes are summarised in Table 9B.

TABLE 9B
Quality profiles of purified sterically masked compounds.
Production titers were determined by Protein A SE-HPLC,
monomer content was assessed by SE-HPLC and CE-SDS.
LC-MS confirmed correct compound identity.

| | | | | | CE-SDS |
|---|---|---|---|---|---|
| Compound | Titer | SE-HPLC | | | % Main |
| ID | (mg/L) | % Monomer | % HMW | % LMW | Peak |
| P1AG8692 | 14.0 | 98 | 2 | 0 | 100 |
| P1AG8693 | 13.0 | 98.1 | 1.9 | 0 | 100 |
| P1AG8694 | 11.0 | 98.2 | 1.8 | 0 | 100 |
| P1AG8695 | 13.0 | 98.8 | 1.2 | 0 | 100 |
| P1AG8696 | 41.0 | 98.9 | 1.1 | 0 | 100 |
| P1AG8697 | 2.0 | 99.4 | 0.6 | 0 | 84.7 |

### 6.3 Blocking Efficiency of Sterically Masked IFNG

To assess the in vitro blocking efficiency of the sterically masked IFNG, HEK Blue IFNG reporter cells were stimulated for 20-24 hours respectively with unmasked IFNG, used as positive control (P1AG8697), and the sterically masked IFNG molecule (P1AG8692). SEAP reporter activity in response to IFNG was measured using a spectrophotometer at 620-655 nm. Obtained results showed that sterically masked IFNG has reduced IFNG activity compared to the positive control (FIG. 10B).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An antigen binding molecule, comprising an antibody that specifically binds to Fibroblast Activation Protein (FAP), and
a homodimer of an interferon gamma (IFNG) variant polypeptide, wherein the IFNG variant polypeptide terminates at the C-terminal end with an amino acid sequence of KRKRP (SEQ ID NO:1).

2. The antigen binding molecule of claim 1, wherein the IFNG variant polypeptide comprises or consists essentially of an amino acid sequence of SEQ ID NO: 2 or SEQ ID NO:3.

3. (canceled)

4. The antigen binding molecule of claim 1, wherein the antibody that specifically binds to FAP comprises:
(a) a heavy chain variable region (V_{H}FAP) comprising a heavy chain complementary determining region (CDR-H1) comprising an amino acid sequence of SEQ ID NO:4, a CDR-H2 comprising an amino acid sequence of SEQ ID NO:5, and a CDR-H3 comprising an amino acid sequence of SEQ ID NO:6, and a light chain variable region (V (FAP) comprising a light chain complementarity determining region (iv) CDR-L1 comprising an amino acid sequence of SEQ ID NO:7, a CDR-L2 comprising an amino acid sequence of SEQ ID NO:8, and a CDR-L3 comprising an amino acid sequence of SEQ ID NO:9, or
(b) a heavy chain variable region (V_{H}FAP) comprising a CDR-H1 comprising an amino acid sequence of SEQ ID NO:12, a CDR-H2 comprising an amino acid sequence of SEQ ID NO: 13, and a CDR-H3 comprising an amino acid sequence of SEQ ID NO:14, and a light chain variable region (V_{L}FAP) comprising a CDR-L1 comprising an amino acid sequence of SEQ ID NO: 15, a CDR-L2 comprising an amino acid sequence of SEQ ID NO:16, and a CDR-L3 comprising an amino acid sequence of SEQ ID NO:17.

5. The antigen binding molecule of claim 4, wherein the antibody that specifically binds to FAP comprises a heavy chain variable region (V_{H}FAP) comprising an amino acid sequence of SEQ ID NO:10 and a light chain variable region (V_{L}FAP) comprising an amino acid sequence of SEQ ID NO:11 or it comprises a heavy chain variable region (V_{H}FAP) comprising an amino acid sequence of SEQ ID NO:18 and a light chain variable region (V_{L}FAP) comprising an amino acid sequence of SEQ ID NO:19.

6. The antigen binding molecule of claim 1, wherein the antigen binding molecule comprises an IgG1 Fc domain or an IgG4 Fc domain and wherein the Fc domain comprises one or more amino acid substitution that reduces the binding affinity of the antibody to an Fc receptor and/or effector function.

7. The antigen binding molecule of claim 1, wherein the Fc domain is of human IgG1 subclass with an amino acid mutations L234A, L235A and P329G (EU numbering according to Kabat EU index).

8. The antigen binding molecule of claim 1, wherein the first IFNG variant polypeptide is fused via a first linker with its N-terminus to the C-terminus of the first heavy chain and second IFNG variant polypeptide is fused via a second linker with its N-terminus to the C-terminus of the second heavy chain.

9. The antigen binding molecule of claim 8, wherein the first and the second linker are peptide linkers.

10. The antigen binding molecule of claim 9, wherein the antigen binding molecule is protease-activatable and comprises a protease recognition site and a masking moiety.

11. The antigen binding molecule of claim 10, wherein the protease recognition site is a substrate for matriptase.

12. The antigen binding molecule of claim 10, wherein the protease recognition site comprises or consists essentially of PQARK (SEQ ID NO:20) or HQARK (SEQ ID NO:21).

13. The antigen binding molecule of claim 10, wherein the protease recognition site is part of a cleavable peptide linker which connects the masking moiety with the IFNG variant polypeptide.

14. The antigen binding molecule of claim 10, wherein the masking moiety is fused at its N-terminus to the C-terminus of the IFNG variant polypeptide via the cleavable peptide linker.

15. The antigen binding molecule of claim 10, wherein the masking moiety is fused at its N-terminus to the C-terminus of the Fc domain via a stable linker and at its C-terminus to the N-terminus of the IFNG variant polypeptide via the cleavable peptide linker.

16. The antigen binding molecule of claim 10, wherein the masking moiety is an antibody or antibody fragment that specifically binds to IFNG.

17. The antigen binding molecule of claim 16, wherein the masking moiety is an scFv that specifically binds to IFNG.

18. The antigen binding molecule of claim 17, wherein the scFv that specifically binds to IFNG comprises:
(a) a heavy chain variable region (V_{H}IFNG) comprising a CDR-H1 comprising an amino acid sequence of SEQ ID NO:22, a CDR-H2 comprising an amino acid sequence of SEQ ID NO: 23, and a CDR-H3 comprising an amino acid sequence of SEQ ID NO:24, and a light chain variable region (V_{L}IFNG) comprising a light chain complementarity determining region (iv) CDR-L1 comprising an amino acid sequence of SEQ ID NO:25, a CDR-L2 comprising an amino acid sequence of SEQ ID NO:26, and a CDR-L3 comprising an amino acid sequence of SEQ ID NO: 27, or
(b) a heavy chain variable region (V_{H}IFNG) comprising a CDR-H1 comprising an amino acid sequence of SEQ ID NO:30, a CDR-H2 comprising an amino acid sequence of SEQ ID NO: 31, and a CDR-H3 comprising an amino acid sequence of SEQ ID NO:32, and a light chain variable region (V_{L}IFNG) comprising a CDR-L1 comprising an amino acid sequence of SEQ ID NO: 33, a CDR-L2 comprising an amino acid sequence of SEQ ID NO:34, and a CDR-L3 comprising an amino acid sequence of SEQ ID NO:35.

19. The bispecific antigen binding molecule of claim 18, wherein the scFv that specifically binds to IFNG comprises (a) a heavy chain variable region (V_{H} IFNG) comprising an amino acid sequence of SEQ ID NO:28 and a light chain variable region (V_{L} IFNG) comprising an amino acid sequence of SEQ ID NO:29, or (b) a heavy chain variable region (V_{H} IFNG) comprising an amino acid sequence of SEQ ID NO:36 and a light chain variable region (V_{L} IFNG) comprising an amino acid sequence of SEQ ID NO: 37.

20. The antigen binding molecule of claim 19, wherein the antigen binding molecule comprises:
(i) two heavy chains comprising an amino acid sequence of SEQ ID NO:40 and two light chains comprising an amino acid sequence of SEQ ID NO:41, or (ii) two heavy chains comprising an amino acid sequence of SEQ ID NO:42 and two light chains comprising an amino acid sequence of SEQ ID NO:41,
(iii) two heavy chains comprising an amino acid sequence of SEQ ID NO:43 and two light chains comprising an amino acid sequence of SEQ ID NO:44, or
(iv) two heavy chains comprising an amino acid sequence of SEQ ID NO:45 and two light chains comprising an amino acid sequence of SEQ ID NO:44.

21. An antigen binding molecule, comprising
(a) an antibody that specifically binds to Fibroblast Activation Protein (FAP) a tumor associated antigen, and
(b) an interferon gamma (IFNG) variant polypeptide, wherein the IFNG variant polypeptide terminates at the C-terminal end with an amino acid sequence of KRKRP (SEQ ID NO:1).

22. The IFNG variant polypeptide of claim 21, wherein the IFNG variant polypeptide comprises or consists essentially of an amino acid sequence of SEQ ID NO: 2 or SEQ ID NO:3.

23.-33. (canceled)

34. An antigen binding molecule comprising:
(a) a homodimer of a first IFNG variant polypeptide and a second IFNG variant polypeptide, wherein the IFNG variant polypeptides each comprise or consist essentially of an amino acid sequence of SEQ ID NO:2; and
(b) an antibody that specifically binds to Fibroblast Activation Protein (FAP antibody) comprising a protease recognition site for matriptase and a masking moiety, and
(i) a heavy chain variable region (VHFAP) comprising a heavy chain complementary determining region (CDR-H1) comprising an amino acid sequence of SEQ ID NO:4, a CDR-H2 comprising an amino acid sequence of SEQ ID NO:5, and a CDR-H3 comprising an amino acid sequence of SEQ ID NO:6, and a light chain variable region (VLFAP) comprising a light chain complementarity determining region (iv) CDR-L1 comprising an amino acid sequence of SEQ ID NO:7, a CDR-L2 comprising an amino acid sequence of SEQ ID NO:8, and a CDR-L3 comprising an amino acid sequence of SEQ ID NO:9, or
(ii) a heavy chain variable region (VHFAP) comprising a CDR-H1 comprising an amino acid sequence of SEQ ID NO:12, a CDR-H2 comprising an amino acid sequence of SEQ ID NO:13, and a CDR-H3 comprising an amino acid sequence of SEQ ID NO: 14, and a light chain variable region (VLFAP) comprising a CDR-L1 comprising an amino acid sequence of SEQ ID NO:15, a CDR-L2 comprising an amino acid sequence of SEQ ID NO:16, and a CDR-L3 comprising an amino acid sequence of SEQ ID NO: 17, or
(iii) a heavy chain variable region (VHFAP) comprising an amino acid sequence of SEQ ID NO: 10 and a light chain variable region (VLFAP) comprising an amino acid sequence of SEQ ID NO:11; or
(iv) a heavy chain variable region (VHFAP) comprising an amino acid sequence of SEQ ID NO: 18 and a light chain variable region (VLFAP) comprising an amino acid sequence of SEQ ID NO:19;
wherein
the protease recognition site for matriptase is part of a cleavable peptide linker that connects the masking moiety with the IFNG variant polypeptide and the masking moiety comprises an antibody or fragment thereof that binds to IFNG (IFNG binder),
the first IFNG variant polypeptide is fused via a first peptide linker with its N-terminus to the C-terminus of the first heavy chain and the second IFNG variant polypeptide is fused via a second peptide linker with its N-terminus to the C-terminus of the second heavy chain; and
the FAP antibody masking moiety is fused at its N-terminus to the C-terminus of the Fc domain via a stable linker and at its C-terminus to the N-terminus of the IFNG variant polypeptide via the cleavable peptide linker.

35. An antigen binding molecule comprising:
(a) a homodimer of a first IFNG variant polypeptide and a second IFNG variant polypeptide, wherein the IFNG variant polypeptides each comprise or consist essentially of an amino acid sequence of SEQ ID NO:3; and
(b) an antibody that specifically binds to Fibroblast Activation Protein (FAP antibody) comprising a protease recognition site for matriptase and a masking moiety, and
(i) a heavy chain variable region (VHFAP) comprising a heavy chain complementary determining region (CDR-H1) comprising an amino acid sequence of SEQ ID NO:4, a CDR-H2 comprising an amino acid sequence of SEQ ID NO:5, and a CDR-H3 comprising an amino acid sequence of SEQ ID NO:6, and a light chain variable region (VLFAP) comprising a light chain complementarity determining region (iv) CDR-L1 comprising an amino acid sequence of SEQ ID NO:7, a CDR-L2 comprising an amino acid sequence of SEQ ID NO:8, and a CDR-L3 comprising an amino acid sequence of SEQ ID NO:9, or
(ii) a heavy chain variable region (VHFAP) comprising a CDR-H1 comprising an amino acid sequence of SEQ ID NO:12, a CDR-H2 comprising an amino acid sequence of SEQ ID NO:13, and a CDR-H3 comprising an amino acid sequence of SEQ ID NO:14, and a light chain variable region (VLFAP) comprising a CDR-L1 comprising an amino acid sequence of SEQ ID NO:15, a CDR-L2 comprising an amino acid sequence of SEQ ID NO:16, and a CDR-L3 comprising an amino acid sequence of SEQ ID NO:17, or
(iii) a heavy chain variable region (VHFAP) comprising an amino acid sequence of SEQ ID NO: 10 and a light chain variable region (VLFAP) comprising an amino acid sequence of SEQ ID NO:11; or
(iv) a heavy chain variable region (VHFAP) comprising an amino acid sequence of SEQ ID NO:18 and a light chain variable region (VLFAP) comprising an amino acid sequence of SEQ ID NO:19;
wherein
the protease recognition site for matriptase is part of a cleavable peptide linker that connects the masking moiety with the IFNG variant polypeptide and the masking moiety comprises an antibody or fragment thereof that binds to IFNG (IFNG binder),
the first IFNG variant polypeptide is fused via a first peptide linker with its N-terminus to the C-terminus of the first heavy chain and the second IFNG variant polypeptide is fused via a second peptide linker with its N-terminus to the C-terminus of the second heavy chain; and
the FAP antibody masking moiety is fused at its N-terminus to the C-terminus of the Fc domain via a stable linker and at its C-terminus to the N-terminus of the IFNG variant polypeptide via the cleavable peptide linker.

36. The antigen binding molecule of claim 34 or 35, wherein
(a) the IFNG binder comprises an scFv and comprises:
(i) a heavy chain variable region (V_{H}IFNG) comprising a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO: 24, and a light chain variable region (V_{L}IFNG) comprising a light chain complementarity determining region (iv) CDR-L1 comprising the amino acid sequence of SEQ ID NO:25, a CDR-L2 comprising the amino acid sequence of SEQ ID NO:26, and a CDR-L3 comprising the amino acid sequence of SEQ ID NO:27, or
(ii) a heavy chain variable region (V_{H}IFNG) comprising a CDR-H1 comprising an amino acid sequence of SEQ ID NO:30, a CDR-H2 comprising an amino acid sequence of SEQ ID NO:31, and a CDR-H3 comprising an amino acid sequence of SEQ ID NO:32, and a light chain variable region (V IFNG) comprising a CDR-L1 comprising an amino acid sequence of SEQ ID NO:33, a CDR-L2 comprising an amino acid sequence of SEQ ID NO: 34, and a CDR-L3 comprising an amino acid sequence of SEQ ID NO:35, or
(iii) a heavy chain variable region (VH IFNG) comprising the amino acid sequence of SEQ ID NO:28 and a light chain variable region (VL IFNG) comprising the amino acid sequence of SEQ ID NO:29, or
(iv) a heavy chain variable region (VH IFNG) comprising the amino acid sequence of SEQ ID NO:36 and a light chain variable region (VL IFNG) comprising the amino acid sequence of SEQ ID NO:37; and/or
(b) the protease recognition site comprises or consists of PQARK (SEQ ID NO:20) or HQARK (SEQ ID NO:21).

37. The antigen binding molecule of claim 35 or 36, wherein the antigen binding molecule comprises:
(a) two heavy chains comprising an amino acid sequence of SEQ ID NO:40 and two light chains comprising an amino acid sequence of SEQ ID NO:41; or
(b) two heavy chains comprising an amino acid sequence of SEQ ID NO:42 and two light chains comprising an amino acid sequence of SEQ ID NO:41; or
(c) two heavy chains comprising an amino acid sequence of SEQ ID NO:43 and two light chains comprising an amino acid sequence of SEQ ID NO:44; or
(d) two heavy chains comprising an amino acid sequence of SEQ ID NO:45 and two light chains comprising an amino acid sequence of SEQ ID NO:44.

38. One or more isolated polynucleotides encoding the antigen binding molecule of claim 1, 34, or 35 the IFNG variant polypeptide of claim 21.

39. An expression vector comprising the one or more isolated polynucleotides of claim 38.

40. A prokaryotic or eukaryotic host cell comprising the one or more isolated polynucleotides of claim 38 or the expression vector of claim 39.

41. A method of producing an antigen binding molecule or IFNG variant polypeptide, comprising the steps of (a) culturing the prokaryotic or eukaryotic host cell of claim 40 under conditions suitable for the expression of the antigen binding molecule or IFNG variant polypeptide and (b) optionally recovering the antigen binding molecule or IFNG variant polypeptide.

42. A pharmaceutical composition comprising the antigen binding molecule of claim 1, 34, or 35 the IFNG variant polypeptide of claim 21 and a pharmaceutically acceptable excipient.

43. A method of treating a disease in an individual, comprising administering to the individual a therapeutically effective amount of the antigen binding molecule of any one of claim 1, 34, or 35 the IFNG variant polypeptide of claim 21 in a pharmaceutically acceptable form.

44. The method of claim 43, wherein said disease is cancer.


SEQUENCE LISTING (166 sequences)

SEQ ID NO: 1 - protein, 5 aa, synthetic construct
  REGION 1..5: note = IFNG C-terminal sequence
KRKRP

SEQ ID NO: 2 - protein, 132 aa, synthetic construct
  REGION 1..132: note = Human IFNG-KRKRP
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 3 - protein, 131 aa, synthetic construct
  REGION 1..131: note = Mouse IFNG-KRKRP
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRP

SEQ ID NO: 4 - protein, 5 aa, synthetic construct
  REGION 1..5: note = FAP(4B9) CDR-H1
SYAMS

SEQ ID NO: 5 - protein, 17 aa, synthetic construct
  REGION 1..17: note = FAP(4B9) CDR-H2
AIIGSGASTYYADSVKG

SEQ ID NO: 6 - protein, 8 aa, synthetic construct
  REGION 1..8: note = FAP(4B9) CDR-H3
GWFGGFNY

SEQ ID NO: 7 - protein, 12 aa, synthetic construct
  REGION 1..12: note = FAP(4B9) CDR-L1
RASQSVTSSYLA

SEQ ID NO: 8 - protein, 7 aa, synthetic construct
  REGION 1..7: note = FAP(4B9) CDR-L2
VGSRRAT

SEQ ID NO: 9 - protein, 9 aa, synthetic construct
  REGION 1..9: note = FAP(4B9) CDR-L3
QQGIMLPPT

SEQ ID NO: 10 - protein, 117 aa, synthetic construct
  REGION 1..117: note = FAP(4B9) VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 11 - protein, 108 aa, synthetic construct
  REGION 1..108: note = FAP(4B9) VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 12 - protein, 5 aa, synthetic construct
  REGION 1..5: note = FAP (28H1) CDR-H1
SHAMS

SEQ ID NO: 13 - protein, 16 aa, synthetic construct
  REGION 1..16: note = FAP (28H1) CDR-H2
AIWASGEQYYADSVKG

SEQ ID NO: 14 - protein, 8 aa, synthetic construct
  REGION 1..8: note = FAP (28H1) CDR-H3
GWLGNFDY

SEQ ID NO: 15 - protein, 12 aa, synthetic construct
  REGION 1..12: note = FAP (28H1) CDR-L1
RASQSVSRSYLA

SEQ ID NO: 16 - protein, 7 aa, synthetic construct
  REGION 1..7: note = FAP (28H1) CDR-L2
GASTRAT

SEQ ID NO: 17 - protein, 9 aa, synthetic construct
  REGION 1..9: note = FAP (28H1) CDR-L3
QQGQVIPPT

SEQ ID NO: 18 - protein, 116 aa, synthetic construct
  REGION 1..116: note = FAP (28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 19 - protein, 108 aa, synthetic construct
  REGION 1..108: note = FAP (28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 20 - protein, 5 aa, synthetic construct
  REGION 1..5: note = Protease recognition site 1
PQARK

SEQ ID NO: 21 - protein, 5 aa, synthetic construct
  REGION 1..5: note = Protease recognition site 2
HQARK

SEQ ID NO: 22 - protein, 5 aa, synthetic construct
  REGION 1..5: note = anti-hu IFNG CDR-H1
SSWIN

SEQ ID NO: 23 - protein, 17 aa, synthetic construct
  REGION 1..17: note = anti-hu IFNG CDR-H2
RIDPSDGEVHYNQDFKD

SEQ ID NO: 24 - protein, 8 aa, synthetic construct
  REGION 1..8: note = anti-hu IFNG CDR-H3
GFLPWFAD

SEQ ID NO: 25 - protein, 11 aa, synthetic construct
  REGION 1..11: note = anti-hu IFNG CDR-L1
KASENVDTYVS

SEQ ID NO: 26 - protein, 7 aa, synthetic construct
  REGION 1..7: note = anti-hu IFNG CDR-L2
GASNRYT

SEQ ID NO: 27 - protein, 9 aa, synthetic construct
  REGION 1..9: note = anti-hu IFNG CDR-L3
GQSYNYPFT

SEQ ID NO: 28 - protein, 117 aa, synthetic construct
  REGION 1..117: note = anti-hu IFNG VH
EVQLVQSGAELKKPGSSVKVSCKASGYIFTSSWINWVKQAPGQCLEWIGRIDPSDGEVHYNQDFKDKATLTVDKSTNTAYMELSSLRSEDTAVYYCARGFLPWFADWGQGTLVTVSS

SEQ ID NO: 29 - protein, 107 aa, synthetic construct
  REGION 1..107: note = anti-hu IFNG VL
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGCGTKVEVK

SEQ ID NO: 30 - protein, 5 aa, synthetic construct
  REGION 1..5: note = anti-mu IFNG CDR-H1
TNSVH

SEQ ID NO: 31 - protein, 16 aa, synthetic construct
  REGION 1..16: note = anti-mu IFNG CDR-H2
AVWTDGTTDYNSALKS

SEQ ID NO: 32 - protein, 8 aa, synthetic construct
  REGION 1..8: note = anti-mu IFNG CDR-H3
EHVYYGLV

SEQ ID NO: 33 - protein, 11 aa, synthetic construct
  REGION 1..11: note = anti-mu IFNG CDR-L1
KASQNVGNNIA

SEQ ID NO: 34 - protein, 7 aa, synthetic construct
  REGION 1..7: note = anti-mu IFNG CDR-L2
YASNRFT

SEQ ID NO: 35 - protein, 9 aa, synthetic construct
  REGION 1..9: note = anti-mu IFNG CDR-L3
QRIFNAPNT

SEQ ID NO: 36 - protein, 117 aa, synthetic construct
  REGION 1..117: note = anti-mu IFNG VH
QVQLQESGPGLVQASQPLSLTCTVSGFSLTTNSVHWIRQPAGKCLEWMGAVWTDGTTDYNSALKSRLTISRDTSKSQVFLEMSSLQTEDVATYYCAREHVYYGLVGWGQGTLVTVSA

SEQ ID NO: 37 - protein, 107 aa, synthetic construct
  REGION 1..107: note = anti-mu IFNG VL
NIVMTQSPKSMSISVGDRVTMNCKASQNVGNNIAWYQQNPGQSPKLLIYYASNRFTGVPDRFTGGGYGTDFTLTINNVQTEDAASYYCQRIFNAPNTFGCGTKLELK

SEQ ID NO: 38 - protein, 239 aa, synthetic construct
  REGION 1..239: note = Anti-hu IFNG scFv
EVQLVQSGAELKKPGSSVKVSCKASGYIFTSSWINWVKQAPGQCLEWIGRIDPSDGEVHYNQDFKDKATLTVDKSTNTAYMELSSLRSEDTAVYYCARGFLPWFADWGQGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGCGTKVEVK

SEQ ID NO: 39 - protein, 239 aa, synthetic construct
  REGION 1..239: note = Anti-mu IFNG scFv
QVQLQESGPGLVQASQPLSLTCTVSGFSLTTNSVHWIRQPAGKCLEWMGAVWTDGTTDYNSALKSRLTISRDTSKSQVFLEMSSLQTEDVATYYCAREHVYYGLVGWGQGTLVTVSAGGGGSGGGGSGGGGSNIVMTQSPKSMSISVGDRVTMNCKASQNVGNNIAWYQQNPGQSPKLLIYYASNRFTGVPDRFTGGGYGTDFTLTINNVQTEDAASYYCQRIFNAPNTFGCGTKLELK

SEQ ID NO: 40 - protein, 877 aa, synthetic construct
  REGION 1..877: note = P1AG7568 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPGGGGSGGGGSGGGPQARKGGGGGGSGGGGGEVQLVQSGAELKKPGSSVKVSCKASGYIFTSSWINWVKQAPGQCLEWIGRIDPSDGEVHYNQDFKDKATLTVDKSTNTAYMELSSLRSEDTAVYYCARGFLPWFADWGQGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGCGTKVEVK

SEQ ID NO: 41 - protein, 215 aa, synthetic construct
  REGION 1..215: note = FAP (4B9) light chain
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 877 aa, synthetic construct
  REGION 1..877: note = P1AG7571 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGGEVQLVQSGAELKKPGSSVKVSCKASGYIFTSSWINWVKQAPGQCLEWIGRIDPSDGEVHYNQDFKDKATLTVDKSTNTAYMELSSLRSEDTAVYYCARGFLPWFADWGQGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGCGTKVEVKGGGGSGGGGSGGGPQARKGGGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 43 - protein, 870 aa, synthetic construct
  REGION 1..870: note = P1AG3766 heavy chain
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGGGGSGGGGSGGGGSGGGGSGGGGSGGGGGHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRPGGGGSGGGGSGGGPQARKGGGGGGSGGGGGQVQLQESGPGLVQASQPLSLTCTVSGFSLTTNSVHWIRQPAGKCLEWMGAVWTDGTTDYNSALKSRLTISRDTSKSQVFLEMSSLQTEDVATYYCAREHVYYGLVGWGQGTLVTVSAGGGGSGGGGSGGGGSNIVMTQSPKSMSISVGDRVTMNCKASQNVGNNIAWYQQNPGQSPKLLIYYASNRFTGVPDRFTGGGYGTDFTLTINNVQTEDAASYYCQRIFNAPNTFGCGTKLELK

SEQ ID NO: 44 - protein, 215 aa, synthetic construct
  REGION 1..215: note = FAP (28H1) mu light chain
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 45 - protein, 870 aa, synthetic construct
  REGION 1..870: note = P1AG3755 heavy chain
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGGGGSGGGGSGGGGSGGGGSGGGGSGGGGGQVQLQESGPGLVQASQPLSLTCTVSGFSLTTNSVHWIRQPAGKCLEWMGAVWTDGTTDYNSALKSRLTISRDTSKSQVFLEMSSLQTEDVATYYCAREHVYYGLVGWGQGTLVTVSAGGGGSGGGGSGGGGSNIVMTQSPKSMSISVGDRVTMNCKASQNVGNNIAWYQQNPGQSPKLLIYYASNRFTGVPDRFTGGGYGTDFTLTINNVQTEDAASYYCQRIFNAPNTFGCGTKLELKGGGGSGGGGSGGGPQARKGGGGGGSGGGGGHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRP

SEQ ID NO: 46 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 47 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 48 - protein, 98 aa, synthetic construct
  REGION 1..98: note = CH1 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 49 - protein, 5 aa, synthetic construct
  REGION 1..5: note = CH1 connector C-terminal end
EPKSC

SEQ ID NO: 50 - protein, 10 aa, synthetic construct
  REGION 1..10: note = Hinge full
  VARIANT 8: note = X is S or P
DKTHTCPXCP

SEQ ID NO: 51 - protein, 7 aa, synthetic construct
  REGION 1..7: note = Hinge middle
  VARIANT 5: note = X is S or P
HTCPXCP

SEQ ID NO: 52 - protein, 5 aa, synthetic construct
  REGION 1..5: note = Hinge short
  VARIANT 3: note = X is S or P
CPXCP

SEQ ID NO: 53 - protein, 107 aa, synthetic construct
  REGION 1..107: note = CH2 domain
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 54 - protein, 105 aa, synthetic construct
  REGION 1..105: note = CH3 domain
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - protein, 330 aa, synthetic construct [sequence omitted]

SEQ ID NO: 56 - protein, 330 aa, synthetic construct
  REGION 1..330: note = Fc IgG1, afroamerican allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 326 aa, synthetic construct
  REGION 1..326: note = Fc IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 58 - protein, 377 aa, synthetic construct
  REGION 1..377: note = Fc IgG3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 59 - protein, 327 aa, synthetic construct
  REGION 1..327: note = Fc IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 61 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 62 - protein, 5 aa, synthetic construct
  REGION 1..5: note = Peptide linker (G4S)
GGGGS

SEQ ID NO: 63 - protein, 10 aa, synthetic construct
  REGION 1..10: note = Peptide linker (G4S)2
GGGGSGGGGS

SEQ ID NO: 64 - protein, 10 aa, synthetic construct
  REGION 1..10: note = Peptide linker (SG4)2
SGGGGSGGGG

SEQ ID NO: 65 - protein, 14 aa, synthetic construct
  REGION 1..14: note = Peptide linker G4(SG4)2
GGGGSGGGGSGGGG

SEQ ID NO: 66 - protein, 15 aa, synthetic construct
  REGION 1..15: note = (G4S)3 peptide linker
GGGGSGGGGSGGGGS

SEQ ID NO: 67 - protein, 20 aa, synthetic construct
  REGION 1..20: note = (G4S)4 peptide linker
GGGGSGGGGSGGGGSGGGGS

SEQ ID NO: 68 - protein, 25 aa, synthetic construct
  REGION 1..25: note = (G4S)5 peptide linker
GGGGSGGGGSGGGGSGGGGSGGGGS

SEQ ID NO: 69 - protein, 30 aa, synthetic construct
  REGION 1..30: note = (G4S)5GGGGG peptide linker
GGGGSGGGGSGGGGSGGGGSGGGGSGGGGG

SEQ ID NO: 70 - protein, 30 aa, synthetic construct
  REGION 1..30: note = PQARK linker
GGGGSGGGGSGGGPQARKGGGGGGSGGGGG

SEQ ID NO: 71 - protein, 30 aa, synthetic construct
  REGION 1..30: note = HQARK linker
GGGGSGGGGSGGGHQARKGGGGGGSGGGGG

SEQ ID NO: 72 - protein, 8 aa, synthetic construct
  REGION 1..8: note = peptide linker
GGGGSGGG

SEQ ID NO: 73 - protein, 7 aa, synthetic construct
  REGION 1..7: note = peptide linker A
LEVLFQG

SEQ ID NO: 74 - protein, 40 aa, synthetic construct
  REGION 1..40: note = peptide linker B
GGGGSGGGGSGGGGSGGGGSGLEVLFQGPGGGGGSGGGGG

SEQ ID NO: 75 - protein, 4 aa, synthetic construct
  REGION 1..4: note = peptide linker C
QARK

SEQ ID NO: 76 - protein, 6 aa, synthetic construct
  REGION 1..6: note = Hinge 1
TCPPCP

SEQ ID NO: 77 - skipped

SEQ ID NO: 78 - protein, 4 aa, synthetic construct
  REGION 1..4: note = KRKR patch
KRKR

SEQ ID NO: 79 - protein, 599 aa, synthetic construct
  REGION 1..599: note = P1AG3768 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 80 - protein, 588 aa, synthetic construct
  REGION 1..588: note = P1AG3769 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTG

SEQ ID NO: 81 - protein, 594 aa, synthetic construct
  REGION 1..594: note = P1AF3570 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQ

SEQ ID NO: 82 - protein, 594 aa, synthetic construct
  REGION 1..594: note = P1AF3571 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSL

SEQ ID NO: 83 - protein, 593 aa, synthetic construct
  REGION 1..593: note = P1AF3574 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 84 - protein, 592 aa, synthetic construct
  REGION 1..592: note = P1AF3575 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKP

SEQ ID NO: 85 - protein, 591 aa, synthetic construct
  REGION 1..591: note = P1AF3576 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRP

SEQ ID NO: 86 - protein, 590 aa, synthetic construct
  REGION 1..590: note = P1AF3577 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKP

SEQ ID NO: 87 - protein, 594 aa, synthetic construct
  REGION 1..594: note = P1AF3578 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSP

SEQ ID NO: 88 - protein, 593 aa, synthetic construct
  REGION 1..593: note = P1AF9470 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKSP

SEQ ID NO: 89 - protein, 592 aa, synthetic construct
  REGION 1..592: note = P1AF3580 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRSP

SEQ ID NO: 90 - protein, 591 aa, synthetic construct
  REGION 1..591: note = P1AF3581 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKSP

SEQ ID NO: 91 - protein, 593 aa, synthetic construct
  REGION 1..593: note = P1AF9464 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGSRKRP

SEQ ID NO: 92 - protein, 593 aa, synthetic construct
  REGION 1..593: note = P1AF9469 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKSKRP

SEQ ID NO: 93 - protein, 593 aa, synthetic construct
  REGION 1..593: note = P1AF9465 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRSRP

SEQ ID NO: 94 - protein, 593 aa, synthetic construct; identical to SEQ ID NO: 88
  REGION 1..593: note = P1AF9470 heavy chain

SEQ ID NO: 95 - protein, 593 aa, synthetic construct
  REGION 1..593: note = P1AF9475 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGPRKRP

SEQ ID NO: 96 - protein, 593 aa, synthetic construct
  REGION 1..593: note = P1AF9476 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKPKRP

SEQ ID NO: 97 - protein, 593 aa, synthetic construct
  REGION 1..593: note = P1AF9477 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRPRP

SEQ ID NO: 98 - protein, 593 aa, synthetic construct
  REGION 1..593: note = P1AF9478 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKPP

SEQ ID NO: 99 - protein, 593 aa, synthetic construct
  REGION 1..593: note = P1AF9483 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGERKRP

SEQ ID NO: 100 - protein, 593 aa, synthetic construct
  REGION 1..593: note = P1AF9484 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKEKRP

SEQ ID NO: 101 - protein, 593 aa, synthetic construct
  REGION 1..593: note = P1AF9485 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRERP

SEQ ID NO: 102 - protein, 593 aa, synthetic construct
  REGION 1..593: note = P1AF9486 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKEP

SEQ ID NO: 103 - protein, 593 aa, synthetic construct
  REGION 1..593: note = P1AF9473 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGSRSRP

SEQ ID NO: 104 - protein, 593 aa, synthetic construct
  REGION 1..593: note = P1AF9481 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGPRPRP

SEQ ID NO: 105 - protein, 593 aa, synthetic construct
  REGION 1..593: note = P1AF9489 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGERERP

SEQ ID NO: 106 - protein, 593 aa, synthetic construct
  REGION 1..593: note = P1AF9474 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKSKSP

SEQ ID NO: 107 - protein, 593 aa, synthetic construct
  REGION 1..593: note = P1AF9482 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKPKPP

SEQ ID NO: 108 - protein, 593 aa, synthetic construct
  REGION 1..593: note = P1AF9490 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKEKEP

SEQ ID NO: 109 - protein, 593 aa, synthetic construct
  REGION 1..593: note = P1AF9471 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGSSKRP

SEQ ID NO: 110 - protein, 593 aa, synthetic construct
  REGION 1..593: note = P1AF9479 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGPPKRP

SEQ ID NO: 111 - protein, 593 aa, synthetic construct
  REGION 1..593: note = P1AF9487 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGEEKRP

SEQ ID NO: 112 - protein, 593 aa, synthetic construct
  REGION 1..593: note = P1AF9472 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRSSP

SEQ ID NO: 113 - protein, 593 aa, synthetic construct
  REGION 1..593: note = P1AF9480 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRPPP

SEQ ID NO: 114 - protein, 593 aa, synthetic construct
  REGION 1..593: note = P1AF9488 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKREEP

SEQ ID NO: 115 - protein, 877 aa, synthetic construct
  REGION 1..877: note = P1AG7567 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPGGGGSGGGGSGGGGSGGGGSGGGGSGGGGGEVQLVQSGAELKKPGSSVKVSCKASGYIFTSSWINWVKQAPGQCLEWIGRIDPSDGEVHYNQDFKDKATLTVDKSTNTAYMELSSLRSEDTAVYYCARGFLPWFADWGQGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGCGTKVEVK

SEQ ID NO: 116 - protein, 877 aa, synthetic construct
  REGION 1..877: note = P1AG7570 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGGEVQLVQSGAELKKPGSSVKVSCKASGYIFTSSWINWVKQAPGQCLEWIGRIDPSDGEVHYNQDFKDKATLTVDKSTNTAYMELSSLRSEDTAVYYCARGFLPWFADWGQGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGCGTKVEVKGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 117 - protein, 608 aa, synthetic construct
  REGION 1..608: note = P1AG1310 heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 118 - protein, 586 aa, synthetic construct
  REGION 1..586: note = P1AG9672 heavy chain
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGGGGSGGGGSGGGGGHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRP

SEQ ID NO: 119 - protein, 617 aa, synthetic construct
  REGION 1..617: note = P1AG8692 heavy chain
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPGGGGSGGGTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 617 aa, synthetic construct
  REGION 1..617: note = P1AG8693 heavy chain
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPLEVLFQGPTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 614 aa, synthetic construct
  REGION 1..614: note = P1AG8694 heavy chain
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPLEVLFQGPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 611 aa, synthetic construct
  REGION 1..611: note = P1AG8695 heavy chain
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPLEVLFQGPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 613 aa, synthetic construct
  REGION 1..613: note = P1AG8696 heavy chain
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRPQARKTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 396 aa, synthetic construct
  REGION 1..396: note = P1AG8697 heavy chain
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGLEVLFQGPGGGGGSGGGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 125 - protein, 15 aa, synthetic construct
  REGION 1..15: note = P1AF3568 IFNG C-terminus
AKTGKRKRSQMLFRG

SEQ ID NO: 126 - protein, 4 aa, synthetic construct
  REGION 1..4: note = P1AF3569 IFNG C-terminus
AKTG

SEQ ID NO: 127 - protein, 10 aa, synthetic construct
  REGION 1..10: note = P1AF3570 IFNG C-terminus
AKTGKRKRSQ

SEQ ID NO: 128 - protein, 10 aa, synthetic construct
  REGION 1..10: note = P1AF3571 IFNG C-terminus
AKTGKRKRSL

SEQ ID NO: 129 - protein, 9 aa, synthetic construct
  REGION 1..9: note = P1AF3574 IFNG C-terminus
AKTGKRKRP

SEQ ID NO: 130 - protein, 8 aa, synthetic construct
  REGION 1..8: note = P1AF3575 IFNG C-terminus
AKTGKRKP

SEQ ID NO: 131 - protein, 7 aa, synthetic construct
  REGION 1..7: note = P1AF3576 IFNG C-terminus
AKTGKRP

SEQ ID NO: 132 - protein, 6 aa, synthetic construct
  REGION 1..6: note = P1AF3577 IFNG C-terminus
AKTGKP

SEQ ID NO: 133 - protein, 10 aa, synthetic construct
  REGION 1..10: note = P1AF3578 IFNG C-terminus
AKTGKRKRSP

SEQ ID NO: 134 - protein, 9 aa, synthetic construct
  REGION 1..9: note = P1AF9470 IFNG C-terminus
AKTGKRKSP

SEQ ID NO: 135 - protein, 8 aa, synthetic construct
  REGION 1..8: note = P1AF3580 IFNG C-terminus
AKTGKRSP

SEQ ID NO: 136 - protein, 7 aa, synthetic construct
  REGION 1..7: note = P1AF3581 IFNG C-terminus
AKTGKSP

SEQ ID NO: 137 - protein, 9 aa, synthetic construct
  REGION 1..9: note = P1AF9464 IFNG C-terminus
AKTGSRKRP

SEQ ID NO: 138 - protein, 9 aa, synthetic construct
  REGION 1..9: note = P1AF9469 IFNG C-terminus
AKTGKSKRP

SEQ ID NO: 139 - protein, 9 aa, synthetic construct
  REGION 1..9: note = P1AF9465 IFNG C-terminus
AKTGKRSRP

SEQ ID NO: 140 - protein, 9 aa, synthetic construct
  REGION 1..9: note = P1AF9475 IFNG C-terminus
AKTGPRKRP

SEQ ID NO: 141 - protein, 9 aa, synthetic construct
  REGION 1..9: note = P1AF9476 IFNG C-terminus
AKTGKPKRP

SEQ ID NO: 142 - protein, 9 aa, synthetic construct
  REGION 1..9: note = P1AF9477 IFNG C-terminus
AKTGKRPRP

SEQ ID NO: 143 - protein, 9 aa, synthetic construct
  REGION 1..9: note = P1AF9478 IFNG C-terminus
AKTGKRPPP

SEQ ID NO: 144 - protein, 9 aa, synthetic construct
  REGION 1..9: note = P1AF9483 IFNG C-terminus
AKTGERKRP

SEQ ID NO: 145 - protein, 9 aa, synthetic construct
  REGION 1..9: note = P1AF9484 IFNG C-terminus
AKTGKEKRP

SEQ ID NO: 146 - protein, 9 aa, synthetic construct
  REGION 1..9: note = P1AF9485 IFNG C-terminus
AKTGKRERP

SEQ ID NO: 147 - protein, 9 aa, synthetic construct
  REGION 1..9: note = P1AF9485 IFNG C-terminus
AKTGKRKEP

SEQ ID NO: 148 - protein, 9 aa, synthetic construct
  REGION 1..9: note = P1AF9473 IFNG C-terminus
AKTGSRSRP

SEQ ID NO: 149 - protein, 9 aa, synthetic construct
  REGION 1..9: note = P1AF9481 IFNG C-terminus
AKTGPRPRP

SEQ ID NO: 150 - protein, 9 aa, synthetic construct
  REGION 1..9: note = P1AF9489 IFNG C-terminus
AKTGERERP

SEQ ID NO: 151 - protein, 9 aa, synthetic construct
  REGION 1..9: note = P1AF9474 IFNG C-terminus
AKTGKSKSP

SEQ ID NO: 152 - protein, 9 aa, synthetic construct
  REGION 1..9: note = P1AF9482 IFNG C-terminus
AKTGKPKPP

SEQ ID NO: 153 - protein, 9 aa, synthetic construct
  REGION 1..9: note = P1AF9490 IFNG C-terminus
AKTGKEKEP

SEQ ID NO: 154 - protein, 9 aa, synthetic construct
  REGION 1..9: note = P1AF9471IFNG C-terminus
AKTGSSKRP

SEQ ID NO: 155 - protein, 9 aa, synthetic construct
  REGION 1..9: note = P1AF9479 IFNG C-terminus
AKTGPPKRP

SEQ ID NO: 156 - protein, 9 aa, synthetic construct
  REGION 1..9: note = P1AF9487 IFNG C-terminus
AKTGEEKRP

SEQ ID NO: 157 - protein, 9 aa, synthetic construct
  REGION 1..9: note = P1AF9472 IFNG C-terminus
AKTGKRSSP

SEQ ID NO: 158 - protein, 9 aa, synthetic construct
  REGION 1..9: note = P1AF9480 IFNG C-terminus
AKTGKRPPP

SEQ ID NO: 159 - protein, 9 aa, synthetic construct
  REGION 1..9: note = P1AF9488 IFNG C-terminus
AKTGKREEP

SEQ ID NO: 160 - protein, 14 aa, synthetic construct
  REGION 1..14: note = IFNG C-terminus shortened by 1 amino acid
AKTGKRKRSQMLFR

SEQ ID NO: 161 - protein, 13 aa, synthetic construct
  REGION 1..13: note = IFNG C-terminus shortened by 2 amino acids
AKTGKRKRSQMLF

SEQ ID NO: 162 - protein, 12 aa, synthetic construct
  REGION 1..12: note = IFNG C-terminus shortened by 3 amino acids
AKTGKRKRSQML

SEQ ID NO: 163 - protein, 11 aa, synthetic construct
  REGION 1..11: note = IFNG C-terminus shortened by 4 amino acids
AKTGKRKRSQM

SEQ ID NO: 164 - protein, 7 aa, synthetic construct
  REGION 1..7: note = IFNG C-terminus shortened by 8 amino acids
AKTGKRK

SEQ ID NO: 165 - protein, 6 aa, synthetic construct
  REGION 1..6: note = IFNG C-terminus shortened by 9 amino acids
AKTGKR

SEQ ID NO: 166 - protein, 5 aa, synthetic construct
  REGION 1..5: note = IFNG C-terminus shortened by 10 amino acids
AKTGK